SEQUENCE LISTING (108 sequences)

SEQ ID NO: 1 - DNA, 1751 bp, Unknown
  source: /note=“Description of Unknown HA_1755_bp_B_Lee_1940 sequence”
atgaaggcaataattgtactactcatggtagtaacatccaatgcagaccgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggattttttgcaacaatggcttgggctgttccaaaggacaactacaaaaatgcaacgaacccacaaacagtggaagtaccatacatttgtacagaaggggaagaccaaattactgtttgggggtttcattcggataacaaaacccaaatgaagagcctctatggagactcaaatcctcaaaagttcacctcatctgctaatggagtgaccacacattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggccggagcaaagtaataaaagggtcattgcctttaattggtgaagcagattgccttcatgaagaatatggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaaaagtacacaagaagctataaacaagataacaaaaaatctcaattctttgagtgaactagaagtaaagaaccttcaaagactaagtggtgccatggatgaactccacaacgaaatacttgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatctgtct

SEQ ID NO: 2 - DNA, 1751 bp, Unknown
  source: /note=“Description of Unknown HA_1755_bp_B_Alabama_02_2018 sequence”
atgaaggcaataattgtactactcatggtagtaacatccaatgcagaccgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactgtgcccggactgtctcaattgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggattttttgcaacaatggcttgggctgttccgaaggacaactataaaaatgcaacgaacccacaaacagtggaagtaccatacatctgtacagaaggggaagaccaaattactgtttgggggtttcattcggataacaaaacccaaatgaagagcctctatggagactcaaatcctcaaaagttcacctcatctgccaatggagtgaccacacattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattgaacaaaagcaagccttactacacaggaaaacatgcaaaagccataggaaattgcccaatatgggtaaaaacacctttgaagcttgccaatggaaccaaatatagacctcctgcaaaactattgaaagaaaggggtttcttcggagctattgctggtttcctagaaggaggatgggaaggaatgattgcaggttggcacggatacacatctcacggagcacatggagtggcagtggcggcagaccttaaaagtacacaagaagctataaacaagataacaaaaaatctcaattctttgagtgaactagaagtaaagaaccttcaaagactaagtggtgccatggatgaactccacaacgaaatacttgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatctgtct

SEQ ID NO: 3 - DNA, 1751 bp, Unknown
  source: /note=“Description of Unknown HA_1755_bp_B_Alabama_04_2018 sequence”
atgaagacaataattgtactactcatggtagtaacatccaatgcagatcgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttgtcaacccaaaacgttatcgatgcagaaaaagcaccaggaggaccctacagacttggaacctcaggatcttgccctaacgctaccagtaaaattggattttttgcaacaatggcttgggctgttccaaaggacaactacaaaaatgcaacgaacccacaaacagtggaagtaccatacatttgtacagaaggggaagaccaaattactgtttgggggttccattcggataacaaaacccaaatgaagagcctctatggagactcaaatcctcaaaagttcacctcatctgctaatggagtgaccacacattatgtttctcagattggcgacttcccagatcaaacagaagacggagggctaccacaaagcggcagaattgttgttgattacatggtgcaaaaacctgggaaaacaggaacaattgtctatcaaaggggtgttttgttgcctcaaaaggtgtggtgcgcgagtggcaggagcaaagtaataaaagggtcattgcctttaattggtgaagcagattgccttcatgaagaatacggtggattaaacaaaagcaagccttactacacaggaaaacatgcaaaagccataggaaattgcccaatatgggtaaaaacacctttgaagcttgctaatggaaccaaatatagacctcctgcaaaactattgaaggaaaggggtttcttcggagctattgctggtttcctagaaggaggatgggaaggaatgattgcaggctggcacggatacacatctcacggagcacatggagtggcagtggcggcagaccttaagagtacacaagaagctataaataagataacaaaaaatctcaattctttgagtgaactagaagtaaagaaccttcaaagactaagtggtgccatggatgaactccacaacgaaatactcgagctggatgaaaaagtggatgacctcagagctgacactataagctcacaaatagaacttgcagtcttgctttccaacgaaggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgactcattgaacattactgctgcatctttaaatgatgatggattggataaccatactatactgctctattactcaactgctgcttctagtttggctgtaacattaatgctagctatttttattgtttatatggtctccagagacaacgtttcatgctccatctgtct

SEQ ID NO: 4 - DNA, 1751 bp, Unknown
  source: /note=“Description of Unknown HA_1755_bp_B_Alabama_05_2018 sequence”
atgaaggcaataattgtactactcatggtagtaacatccaatgcagaccgaatctgcactgggataacatcttcaaactcacctcatgtggtcaaaacagctactcaaggggaggtcaatgtgactggcgtgataccactgacaacaacaccaacaaaatcttattttgcaaatctcaaaggaacaaggaccagagggaaactatgcccggactgtctcaactgtacagatctggatgtggccttgggcaggccaatgtgtgtggggaccacaccttctgctaaagcttcaatactccatgaggtcagacctgttacatccgggtgctttcctataatgcacgacagaacaaaaatcagacaactacccaatcttctcagaggatatgaaaagatcaggttatcaacccaaaacgttatcgatgcagaaaaagcaccaggaggaccctacagacttggaacctcaggatcttgccctaacgctaccagtaaaattggattttttgcaacaatggcttgggctgttccaaaggacaactacaaaaatgcaacgaacccacaaacagtggaagtaccatacatttgtacagaaggggaagaccaaattactgtttgggggtttcattcggataacaaaacccaaatgaagagcctctatggagactcaaatcctcaaaagttcacctcatctgctaatggagtgaccacacattatgtttctcagattggcgacttcccagatcaaacagaagacggaggactaccacaaagcggcagaattgttgttgattacatggtgcaaaaacctgggaaaacaggaacaattgtctatcaaaggggtgttttgttgcctcaaaaggtgtggtgcgcgagtggccggagcaaagtaataaaagggtcattgcctttaattggtgaagcagattgccttcatgaagaatatggtggattaaacaaaagcaagccttactacacaggaaaacatgcaaaagccataggaaattgcccaatatgggtaaaaacacctttgaagcttgccaatggaaccaaatatagacctcctgcaaaactattgaaggaaaggggtttcttcggagctattgctggtttcctagaaggaggatgggaaggaatgattgcaggttggcacggatacacatctcacggagcacatggagtggcagtggcggcagaccttaaaagtacacaagaagctataaacaagataacaaaaaatctcaattctttgagtgaactagaagtaaagaaccttcaaagactaagtggtgccatggatgaactccacaacgaaataattgagctggatgaaaaagtggatgacctcagagctgacactataagctcacaaatagaacttgcagtcttgctttccaacgagggaataataaacagtgaagatgagcatctattggcacttgagagaaaactaaagaaaatgctgggtccctctgctgtagacataggaaacggatgcttcgaaaccaaacacaaatgcaaccagacctgcttagacaggatagctgctggcacctttaatgcaggagaattttctctccccacttttgactcattgaacattactgctgcatctttaaatgatgatgggttggataaccatactatactgctctattactcaactgctgcttctagtttggctgtaacattaatgctagctatttttattgtttatatggtctccagagacaacgtttcatgctccatctgtct

SEQ ID NO: 5 - protein, 584 aa, Unknown
  source: /note=“Description of Unknown HA_1755_bp_B_Lee_1940 sequence”
MKAIIVLLMVVTSNADRICTGITSSNSPHVVKTATQGEVNVTGVIPLTTTPTKSHFANLKGTQTRGKLCPNCFNCTDLDVALGRPKCMGNTPSAKVSILHEVKPATSGCFPIMHDRTKIRQLPNLLRGYENIRLSTSNVINTETAPGGPYKVGTSGSCPNVANGNGFFNTMAWVIPKDNNKTAINPVTVEVPYICSEGEDQITVWGFHSDDKTQMERLYGDSNPQKFTSSANGVTTHYVSQIGGFPNQTEDEGLKQSGRIVVDYMVQKPGKTGTIVYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNYLSELEVKNLQRLSGAMNELHDEILELDEKVDDLRADTISSQIELAVLLSNEGIINSEDEHLLALERKLKKMLGPSAVEIGNGCFETKHKCNQTCLDRIAAGTFNAGDFSLPTFDSLNITAASLNDDGLDNHTILLYYSTAASSLAVTLMIAIFIVYMVSRDNVSCSICL

SEQ ID NO: 6 - protein, 583 aa, Unknown
  source: /note=“Description of Unknown HA_1755_bp_B_Alabama_02_2018 sequence”
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSIC

SEQ ID NO: 7 - protein, 583 aa, Unknown
  source: /note=“Description of Unknown HA_1755_bp_B_Alabama_04_2018 sequence”
MKTIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSIC

SEQ ID NO: 8 - protein, 583 aa, Unknown
  source: /note=“Description of Unknown HA_1755_bp_B_Alabama_05_2018 sequence”
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEIIELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSIC

SEQ ID NO: 9 - protein, 14 aa, Unknown
  source: /note=“Description of Unknown Hepatitis B amino acid sequence”
MKAIIVLLMVVTSN

SEQ ID NO: 10 - protein, 14 aa, Unknown
  source: /note=“Description of Unknown Hepatitis B amino acid sequence”
MKAIIVLLMVVTSN

SEQ ID NO: 11 - protein, 14 aa, Unknown
  source: /note=“Description of Unknown Hepatitis B amino acid sequence”
MKAIIVLLMVVTSS

SEQ ID NO: 12 - protein, 14 aa, Unknown
  source: /note=“Description of Unknown Hepatitis B amino acid sequence”
MKAIIVLLMVVTSN

SEQ ID NO: 13 - protein, 14 aa, Unknown
  source: /note=“Description of Unknown Hepatitis B amino acid sequence”
MKAIIVLLMVVTSN

SEQ ID NO: 14 - protein, 14 aa, Unknown
  source: /note=“Description of Unknown Hepatitis B amino acid sequence”
MKAIIVLLMVVTSN

SEQ ID NO: 15 - protein, 14 aa, Unknown
  source: /note=“Description of Unknown Hepatitis B amino acid sequence”
MKAIIVLLMVVTSD

SEQ ID NO: 16 - protein, 14 aa, Unknown
  source: /note=“Description of Unknown Hepatitis B amino acid sequence”
MKAIIVLLMVVTSN

SEQ ID NO: 17 - protein, 14 aa, Unknown
  source: /note=“Description of Unknown Hepatitis B amino acid sequence”
MKAIIVLLMVVTSN

SEQ ID NO: 18 - protein, 14 aa, Unknown
  source: /note=“Description of Unknown Hepatitis B amino acid sequence”
MKAIIVLLMVVTSN

SEQ ID NO: 19 - protein, 14 aa, Unknown
  source: /note=“Description of Unknown Hepatitis B amino acid sequence”
MKAIIVLLMVVTSN

SEQ ID NO: 20 - protein, 14 aa, Unknown
  source: /note=“Description of Unknown Hepatitis B amino acid sequence”
MKAIIVLLMVVTSN

SEQ ID NO: 21 - protein, 584 aa, Unknown; identical to SEQ ID NO: 5
  source: /note=“Description of Unknown Lee/1940 sequence”

SEQ ID NO: 22 - protein, 345 aa, Unknown
  source: /note=“Description of Unknown Russia/1960 sequence”
DRICTGITSSNSPHVVKTATQGEVNVTGVIPLTTTPTKSHFANLKGTQTRGKLCPNCLNCTDLDVALGRPKCSGTIPSAKVSILHEVKPVTSGCFPIMHDRTKIRQLPNLLRGYENIRLSTRNVINAETAPGGPYTVGTSGSCPNVTNGKGFFETMAWAVPKNKNKTATNPLTVEVPYICTKGEDQITVWGFHSDDETQMVILYGDSKPQKFTSSANGVTTHYVSQIGGFPNQTEDEGLKQSGRIVVDYIVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKE

SEQ ID NO: 23 - protein, 581 aa, Unknown
  source: /note=“Description of Unknown HongKong/1972 sequence”
MKAIIVLLMVVTSNADRICTGITSSNSPHVVKTATQGEVNVTGVIPLTTTPTKSHFANLKGTQTRGKLCPNCLNCTDLDVALGRPKCMGTIPSAKASILHEVKPVTSGCFPIMHDRTKIRQLPNLLRGYENIRLSARNVINAETAPGGPYIVGISGSCPNVTNGNGFFATMAWAVPKNKTATNPLTVEVPYICAKGEDQITVWGFHSDNEIQMVKLYGDSKPQKFTSSANGVTTHYVSQIGGFPNQAEDEGLPQSGRIVVDYMVQKPGKTGTIAYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSIC

SEQ ID NO: 24 - protein, 582 aa, Unknown
  source: /note=“Description of Unknown Singapore/1979 sequence”
MKAIIVLLMVVTSNADRICTGITSSNSPHVVKTATQGEVNVTGVIPLTTTPTKSHFANLKGTKTRGKLCPNCLNCTDLDVALGRPKCMGTIPSAKASILHEVKPVTSGCFPIMHDRTKIRQLPNLLRGYENIRLSTRNVINAERAPGGPYIIGTSGSCPNVTNGNGFFATMAWAVPKDNKTATNPLTVEVPYICTKGEDQITVWGFHSDTETQMVKLYGDSKPQKFTSSANGVTTHYVSQIGGFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSIC

SEQ ID NO: 25 - protein, 582 aa, Unknown
  source: /note=“Description of Unknown Yamagata/1988 sequence”
MKAIIVLLMVVTSNADRICTGITSSNSPHVVKTATQGEVNVTGVIPLTTTPTKSHFANLKGTKTRGKLCPNCLNCTDLDVALGRPMCMGTIPSAKASILHEVRPVTSGCFPIMHDRTKIRQLPNLLRGYENIRLSTHNVINAERAPGGPYRLGTSGSCPNVTSRNGFFATMAWAVPRDNKTATNPLTVEVPYICTKGEDQITVWGFHSDDKTQMKNLYGDSNPQKFTSSANGVTTHYVSQIGDFPNQTEDGGLPQSGRIVVDYMVQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMIAIFIVYMVSRDNVSCSIC

SEQ ID NO: 26 - protein, 584 aa, Unknown
  source: /note=“Description of Unknown Malaysia/2004 sequence”
  MOD_RES (214)..(214): Any amino acid
MKAIIVLLMVVTSNADRICTGITSSNSPHVVKTATQGEVNVTGVIPLTTTPTKSHFANLKGTETRGKLCPKCLNCTDLDVALGRPKCTGNIPSARVSILHEVRPVTSGCFPIMHDRTKIRQLPNLLRGYEHIRLSTHNVINAENAPGGPYKIGTSGSCPNVTNGNGFFATMAWAVPKNDNNKTATNSLTIEVPYICTEGEDQITVWGFHSDNEXQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSIC

SEQ ID NO: 27 - protein, 583 aa, Unknown
  source: /note=“Description of Unknown Florida/2006 sequence”
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVKPVTSGCFPIMHDRTKIRQLPNLLRGYENIRLSTQNVIDAEKAPGGPYRLGTSGSCPNATSKSGFFATMAWAVPKDNNKNATNPLTVEVPYICTEGEDQITVWGFHSDDKTQMKNLYGDSNPQKFTSSANGVTTHYVSQIGSFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFNAGEFSLPTFDSLNITAASLNDDGLDNHTILLYYSTAASSLAVTLMLAIFIVYMVSRDNVSCSIC

SEQ ID NO: 28 - protein, 585 aa, Unknown
  source: /note=“Description of Unknown FluB/2018 & 2019 consensus sequence ”
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNKYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 29 - protein, 120 aa, Artificial Sequence
  source: /note=“Description of Artificial Sequence Synthetic polypeptide”
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSS

SEQ ID NO: 30 - protein, 8 aa, Artificial Sequence
  source: /note=“Description of Artificial Sequence Synthetic peptide”
GFTFSDYY

SEQ ID NO: 31 - protein, 8 aa, Artificial Sequence
  source: /note=“Description of Artificial Sequence Synthetic peptide”
ITYSGSTI

SEQ ID NO: 32 - protein, 13 aa, Artificial Sequence
  source: /note=“Description of Artificial Sequence Synthetic peptide”
ARDRGTTMVPFDY

SEQ ID NO: 33 - protein, 107 aa, Artificial Sequence
  source: /note=“Description of Artificial Sequence Synthetic polypeptide”
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIK

SEQ ID NO: 34 - protein, 6 aa, Artificial Sequence
  source: /note=“Description of Artificial Sequence Synthetic peptide”
QDITNY

SEQ ID NO: 35 - protein, 3 aa, Artificial Sequence
  source: /note=“Description of Artificial Sequence Synthetic peptide”
AAS

SEQ ID NO: 36 - protein, 9 aa, Artificial Sequence
  source: /note=“Description of Artificial Sequence Synthetic peptide”
QQYDNLPLT

SEQ ID NO: 37 - protein, 450 aa, Artificial Sequence
  source: /note=“Description of Artificial Sequence Synthetic polypeptide”
QVQLVESGGGLVKPGGSLRLSCAASGFTFSDYYMSWIRQAPGKGLEWVSYITYSGSTIYYADSVKGRFTISRDNAKSSLYLQMNSLRAEDTAVYYCARDRGTTMVPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 214 aa, Artificial Sequence
  source: /note=“Description of Artificial Sequence Synthetic polypeptide”
DIQMTQSPSSLSASVGDRVTITCQASQDITNYLNWYQQKPGKAPKLLIYAASNLETGVPSRFSGSGSGTDFTFTISGLQPEDIATYYCQQYDNLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - DNA, 360 bp, Artificial Sequence
  source: /note=“Description of Artificial Sequence Synthetic polynucleotide”
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctca

SEQ ID NO: 40 - DNA, 24 bp, Artificial Sequence
  source: /note=“Description of Artificial Sequence Synthetic oligonucleotide”
ggattcaccttcagtgactactac

SEQ ID NO: 41 - DNA, 24 bp, Artificial Sequence
  source: /note=“Description of Artificial Sequence Synthetic oligonucleotide”
attacttatagtggtagtaccata

SEQ ID NO: 42 - DNA, 39 bp, Artificial Sequence
  source: /note=“Description of Artificial Sequence Synthetic oligonucleotide”
gcgagagatcgcggtacaactatggtcccctttgactac

SEQ ID NO: 43 - DNA, 321 bp, Artificial Sequence
  source: /note=“Description of Artificial Sequence Synthetic polynucleotide”
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaa

SEQ ID NO: 44 - DNA, 18 bp, Artificial Sequence
  source: /note=“Description of Artificial Sequence Synthetic oligonucleotide”
caggacattaccaactat

SEQ ID NO: 45 - DNA, 9 bp, Artificial Sequence
  source: /note=“Description of Artificial Sequence Synthetic oligonucleotide”
gctgcatcc

SEQ ID NO: 46 - DNA, 27 bp, Artificial Sequence
  source: /note=“Description of Artificial Sequence Synthetic oligonucleotide”
caacagtatgataatctccctctcact

SEQ ID NO: 47 - DNA, 1353 bp, Artificial Sequence
  source: /note=“Description of Artificial Sequence Synthetic polynucleotide”
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattacttatagtggtagtaccatatactacgcagactctgtgaagggccgattcaccatctccagggacaacgccaagagctcactgtatctgcaaatgaacagcctgagagccgaggacacggccgtgtattactgtgcgagagatcgcggtacaactatggtcccctttgactactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 48 - DNA, 645 bp, Artificial Sequence
  source: /note=“Description of Artificial Sequence Synthetic polynucleotide”
gacatccagatgacccagtctccatcctccctgtctgcatctgtaggagacagagtcaccatcacttgccaggcgagtcaggacattaccaactatttaaattggtatcagcagaaaccagggaaagcccctaagctcctgatctacgctgcatccaatttggaaacaggggtcccatcaaggttcagtggaagtggatctgggacagattttactttcaccatcagcggcctgcagcctgaagatattgcaacatattactgtcaacagtatgataatctccctctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 49 - protein, 120 aa, Artificial Sequence
  source: /note=“Description of Artificial Sequence Synthetic polypeptide”
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSS

SEQ ID NO: 50 - protein, 8 aa, Artificial Sequence
  source: /note=“Description of Artificial Sequence Synthetic peptide”
GITFSNAW

SEQ ID NO: 51 - protein, 10 aa, Artificial Sequence
  source: /note=“Description of Artificial Sequence Synthetic peptide”
IKSKTDGGTT

SEQ ID NO: 52 - protein, 11 aa, Artificial Sequence
  source: /note=“Description of Artificial Sequence Synthetic peptide”
TTARWDWYFDL

SEQ ID NO: 53 - protein, 107 aa, Artificial Sequence
  source: /note=“Description of Artificial Sequence Synthetic polypeptide”
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIK

SEQ ID NO: 54 - protein, 6 aa, Artificial Sequence
  source: /note=“Description of Artificial Sequence Synthetic peptide”
QDIWNY

SEQ ID NO: 55 - protein, 3 aa, Artificial Sequence
  source: /note=“Description of Artificial Sequence Synthetic peptide”
DAS

SEQ ID NO: 56 - protein, 9 aa, Artificial Sequence
  source: /note=“Description of Artificial Sequence Synthetic peptide”
QQHDDLPPT

SEQ ID NO: 57 - protein, 450 aa, Artificial Sequence
  source: /note=“Description of Artificial Sequence Synthetic polypeptide”
EVQLVESGGGLVKPGGSLRLSCAASGITFSNAWMSWVRQAPGKGLEWVGRIKSKTDGGTTDYAAPVKGRFTISRDDSKNTLYLQMNSLKTEDTAVYYCTTARWDWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, Artificial Sequence
  source: /note=“Description of Artificial Sequence Synthetic polypeptide”
DIQMTQSPSSLSASVGDRVTITCQASQDIWNYINWYQQKPGKAPKLLIYDASNLKTGVPSRFSGSGSGTDFTFTISSLQPEDIATYYCQQHDDLPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - DNA, 360 bp, Artificial Sequence
  source: /note=“Description of Artificial Sequence Synthetic polynucleotide”
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctca

SEQ ID NO: 60 - DNA, 24 bp, Artificial Sequence
  source: /note=“Description of Artificial Sequence Synthetic oligonucleotide”
ggaatcactttcagtaacgcctgg

SEQ ID NO: 61 - DNA, 30 bp, Artificial Sequence
  source: /note=“Description of Artificial Sequence Synthetic oligonucleotide”
attaaaagcaaaactgatggtgggacaaca

SEQ ID NO: 62 - DNA, 33 bp, Artificial Sequence
  source: /note=“Description of Artificial Sequence Synthetic oligonucleotide”
accacagcgaggtgggactggtacttcgatctc

SEQ ID NO: 63 - DNA, 321 bp, Artificial Sequence
  source: /note=“Description of Artificial Sequence Synthetic polynucleotide”
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaa

SEQ ID NO: 64 - DNA, 18 bp, Artificial Sequence
  source: /note=“Description of Artificial Sequence Synthetic oligonucleotide”
caggacatttggaattat

SEQ ID NO: 65 - DNA, 9 bp, Artificial Sequence
  source: /note=“Description of Artificial Sequence Synthetic oligonucleotide”
gatgcatcc

SEQ ID NO: 66 - DNA, 27 bp, Artificial Sequence
  source: /note=“Description of Artificial Sequence Synthetic oligonucleotide”
caacagcatgatgatctccctccgacc

SEQ ID NO: 67 - DNA, 1353 bp, Artificial Sequence
  source: /note=“Description of Artificial Sequence Synthetic polynucleotide”
gaggtgcagctggtggagtctgggggaggcttggtaaagcctggggggtcccttagactctcctgtgcagcctctggaatcactttcagtaacgcctggatgagttgggtccgccaggctccagggaaggggctggagtgggttggccgtattaaaagcaaaactgatggtgggacaacagactacgccgcacccgtgaaaggcagattcaccatctcaagagatgattcaaaaaacacgctgtatctacaaatgaacagcctgaaaaccgaggacacagccgtgtattactgtaccacagcgaggtgggactggtacttcgatctctggggccgtggcaccctggtcactgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 68 - DNA, 645 bp, Artificial Sequence
  source: /note=“Description of Artificial Sequence Synthetic polynucleotide”
gacatccagatgacccagtctccatcctccctgtctgcatctgtaggagacagagtcaccatcacttgccaggcgagtcaggacatttggaattatataaattggtatcagcagaaaccagggaaggcccctaagctcctgatctacgatgcatccaatttgaaaacaggggtcccatcaaggttcagtggaagtggatctgggacagattttactttcaccatcagcagcctgcagcctgaagatattgcaacatattactgtcaacagcatgatgatctccctccgaccttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 69 - protein, 120 aa, Artificial Sequence
  source: /note=“Description of Artificial Sequence Synthetic polypeptide”
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSS

SEQ ID NO: 70 - protein, 8 aa, Artificial Sequence
  source: /note=“Description of Artificial Sequence Synthetic peptide”
GFTFSNYA

SEQ ID NO: 71 - protein, 8 aa, Artificial Sequence
  source: /note=“Description of Artificial Sequence Synthetic peptide”
ISYDGSNK

SEQ ID NO: 72 - protein, 13 aa, Artificial Sequence
  source: /note=“Description of Artificial Sequence Synthetic peptide”
ASGSDYGDYLLVY

SEQ ID NO: 73 - protein, 110 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVL

SEQ ID NO: 74 - protein, 9 aa, Artificial Sequence
  source: /note=“Description of Artificial Sequence Synthetic peptide”
SSDVGGYNY

SEQ ID NO: 75 - protein, 3 aa, Artificial Sequence
  source: /note=“Description of Artificial Sequence Synthetic peptide”
DVS

SEQ ID NO: 76 - protein, 10 aa, Artificial Sequence
  source: /note=“Description of Artificial Sequence Synthetic peptide”
NSLTSISTWV

SEQ ID NO: 77 - protein, 450 aa, Artificial Sequence
  source: /note=“Description of Artificial Sequence Synthetic polypeptide”
QVQLVESGGGVVQPGRSLRLSCAASGFTFSNYAMYWVRQAPGKGLEWVAVISYDGSNKYYADSVKGRFTISRDNSKNTLYLQMNSLRTEDTAVYYCASGSDYGDYLLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 216 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYDVSKRPSGVSNRFSGSKSGNTASLTISGLQSEDEADYYCNSLTSISTWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 79 - DNA, 360 bp, Artificial Sequence
  source: /note=“Description of Artificial Sequence Synthetic polynucleotide”
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctca

SEQ ID NO: 80 - DNA, 24 bp, Artificial Sequence
  source: /note=“Description of Artificial Sequence Synthetic oligonucleotide”
ggattcaccttcagtaactatgct

SEQ ID NO: 81 - DNA, 24 bp, Artificial Sequence
  source: /note=“Description of Artificial Sequence Synthetic oligonucleotide”
atatcatatgatggaagtaataaa

SEQ ID NO: 82 - DNA, 39 bp, Artificial Sequence
  source: /note=“Description of Artificial Sequence Synthetic oligonucleotide”
gcgagtggctccgactacggtgactacttattggtttac

SEQ ID NO: 83 - DNA, 330 bp, Artificial Sequence
  source: /note=“Description of Artificial Sequence Synthetic polynucleotide”
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtccta

SEQ ID NO: 84 - DNA, 27 bp, Artificial Sequence
  source: /note=“Description of Artificial Sequence Synthetic oligonucleotide”
agcagtgacgttggtggttataactat

SEQ ID NO: 85 - DNA, 9 bp, Artificial Sequence
  source: /note=“Description of Artificial Sequence Synthetic oligonucleotide”
gatgtcagt

SEQ ID NO: 86 - DNA, 30 bp, Artificial Sequence
  source: /note=“Description of Artificial Sequence Synthetic oligonucleotide”
aactctttgacaagcatcagcacttgggtg

SEQ ID NO: 87 - DNA, 1353 bp, Artificial Sequence
  source: /note=“Description of Artificial Sequence Synthetic polynucleotide”
caggtgcagctggtggagtctgggggaggcgtggtccagcctgggaggtccctgagactctcctgtgcagcctctggattcaccttcagtaactatgctatgtactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagaactgaggacacggctgtgtattactgtgcgagtggctccgactacggtgactacttattggtttactggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 88 - DNA, 651 bp, Artificial Sequence
  source: /note=“Description of Artificial Sequence Synthetic polynucleotide”
cagtctgccctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgatgtcagtaagcggccctcaggggtttctaatcgcttctctggctccaagtctggcaacacggcctccctgaccatctctgggctccagtctgaggacgaggctgattattactgcaactctttgacaagcatcagcacttgggtgttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 89 - protein, 123 aa, Artificial Sequence
  source: /note=“Description of Artificial Sequence Synthetic polypeptide”
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSS

SEQ ID NO: 90 - protein, 8 aa, Artificial Sequence
  source: /note=“Description of Artificial Sequence Synthetic peptide”
GYIFTGYY

SEQ ID NO: 91 - protein, 8 aa, Artificial Sequence
  source: /note=“Description of Artificial Sequence Synthetic peptide”
INPNSGGA

SEQ ID NO: 92 - protein, 16 aa, Artificial Sequence
  source: /note=“Description of Artificial Sequence Synthetic peptide”
ARGSRYDWNQNNWFDP

SEQ ID NO: 93 - protein, 110 aa, Artificial Sequence
  source: /note=“Description of Artificial Sequence Synthetic polypeptide”
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVL

SEQ ID NO: 94 - protein, 9 aa, Artificial Sequence
  source: /note=“Description of Artificial Sequence Synthetic peptide”
SSDVGTYNY

SEQ ID NO: 95 - protein, 10 aa, Artificial Sequence
  source: /note=“Description of Artificial Sequence Synthetic peptide”
SSFTTSSTVV

SEQ ID NO: 96 - protein, 453 aa, Artificial Sequence
  source: /note=“Description of Artificial Sequence Synthetic polypeptide”
QVQLVQSGAEVKKPGASVKVSCKASGYIFTGYYMHWVRQAPGQGLEWMGWINPNSGGANYAQKFQGRVTLTRDTSITTVYMELSRLRFDDTAVYYCARGSRYDWNQNNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 216 aa, Artificial Sequence
  source: /note=“Description of Artificial Sequence Synthetic polypeptide”
QSALTQPASVSGSPGQSITISCTGTSSDVGTYNYVSWYQQHPGKAPKLMIFDVSNRPSGVSDRFSGSKSGNTASLTISGLQAEDEADYYCSSFTTSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 98 - DNA, 369 bp, Artificial Sequence
  source: /note=“Description of Artificial Sequence Synthetic polynucleotide”
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctca

SEQ ID NO: 99 - DNA, 24 bp, Artificial Sequence
  source: /note=“Description of Artificial Sequence Synthetic oligonucleotide”
ggatacatcttcaccggctactat

SEQ ID NO: 100 - DNA, 24 bp, Artificial Sequence
  source: /note=“Description of Artificial Sequence Synthetic oligonucleotide”
atcaaccctaacagtggtggcgca

SEQ ID NO: 101 - DNA, 48 bp, Artificial Sequence
  source: /note=“Description of Artificial Sequence Synthetic oligonucleotide”
gcgagaggatcccggtatgactggaaccagaacaactggttcgacccc

SEQ ID NO: 102 - DNA, 330 bp, Artificial Sequence
  source: /note=“Description of Artificial Sequence Synthetic polynucleotide”
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtccta

SEQ ID NO: 103 - DNA, 27 bp, Artificial Sequence
  source: /note=“Description of Artificial Sequence Synthetic oligonucleotide”
agcagtgacgttggtacttataactat

SEQ ID NO: 104 - DNA, 9 bp, Artificial Sequence
  source: /note=“Description of Artificial Sequence Synthetic oligonucleotide”
gatgtcagt

SEQ ID NO: 105 - DNA, 30 bp, Artificial Sequence
  source: /note=“Description of Artificial Sequence Synthetic oligonucleotide”
agctcatttacaaccagcagcactgtggtt

SEQ ID NO: 106 - DNA, 1362 bp, Artificial Sequence
  source: /note=“Description of Artificial Sequence Synthetic polynucleotide”
caggtgcagctggtgcagtctggggctgaggtgaagaagcctggggcctcagtgaaggtctcctgcaaggcttctggatacatcttcaccggctactatatgcactgggtgcgacaggcccctggacaggggcttgagtggatgggatggatcaaccctaacagtggtggcgcaaactatgcacagaagtttcagggcagggtcaccctgaccagggacacgtccatcaccacagtctacatggaactgagcaggctgagatttgacgacacggccgtgtattactgtgcgagaggatcccggtatgactggaaccagaacaactggttcgacccctggggccagggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagtccctctccctgtctccgggtaaatga

SEQ ID NO: 107 - DNA, 651 bp, Artificial Sequence
  source: /note=“Description of Artificial Sequence Synthetic polynucleotide”
cagtctgccctgactcagcctgcctccgtgtctgggtctcctggacagtcgatcaccatctcctgcactggaaccagcagtgacgttggtacttataactatgtctcctggtaccaacaacacccaggcaaagcccccaaactcatgatttttgatgtcagtaatcggccctcaggggtttctgatcgcttctctggctccaagtctggcaacacggcctccctgaccatctctgggctccaggctgaggacgaggctgattattactgcagctcatttacaaccagcagcactgtggttttcggcggagggaccaagctgaccgtcctaggccagcccaaggccgccccctccgtgaccctgttccccccctcctccgaggagctgcaggccaacaaggccaccctggtgtgcctgatctccgacttctaccccggcgccgtgaccgtggcctggaaggccgactcctcccccgtgaaggccggcgtggagaccaccaccccctccaagcagtccaacaacaagtacgccgcctcctcctacctgtccctgacccccgagcagtggaagtcccaccggtcctactcctgccaggtgacccacgagggctccaccgtggagaagaccgtggcccccaccgagtgctcctga

SEQ ID NO: 108 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2021/0142868 A1
(43) Pub. Date: May 13, 2021

(54) METHODS AND SYSTEMS FOR IDENTIFYING, CLASSIFYING, AND/OR RANKING GENETIC SEQUENCES

(71) Applicant: Regeneron Pharmaceuticals, Inc., Tarrytown, NY (US)

(72) Inventors: Richard Copin, Tarrytown, NY (US); Wei Keat Lim, Tarrytown, NY (US)

(21) Appl. No.: 17/095,562

(22) Filed: Nov. 11, 2020

Related U.S. Application Data
(60) Provisional application No. 62/993,567, filed on Mar. 23, 2020.
(60) Provisional application No. 62/934,323, filed on Nov. 12, 2019.

(51) Int. Cl. G16B 30/10; G16B 30/20; C12Q 1/6869; C12Q 1/70

(52) U.S. Cl. CPC G16B 30/10; C12Q 1/70; C12Q 1/6869; G16B 30/20

(57) ABSTRACT

The present disclosure provides methods and systems for analysis of genomic sequence information. The present disclosure provides, among other things, methods and systems for characterizing sequence conservation. As is discussed herein, certain methods and systems of the present disclosure include assignment of a similarity score to a sequence or pairwise sequence comparison based on a measure of coverage and a measure of identity between two aligned sequences.

### CROSS-REFERENCE TO RELATED APPLICATIONS

This application claims the benefit of U.S. Provisional Patent Application No. 62/993,567, filed on Mar. 23, 2020, and U.S. Provisional Patent Application No. 62/934,323, filed on Nov. 12, 2019, the disclosure of each of which is hereby incorporated by reference in its entirety.

### SEQUENCE LISTING

A Sequence Listing in the form of a text file (entitled “2010794 2132 SL”, created on Nov. 10, 2020, and having a size of 146,610 bytes) is incorporated herein by reference in its entirety.

### BACKGROUND

The speed and efficiency of genome sequencing have increased dramatically in recent decades, enabling the collection of enormous amounts of genomic sequence information. More than one million genomic sequences are available in publicly accessible databases, the bulk of which are microbial genomes. For instance, approximately 160,000 genomic sequences have been deposited in publicly accessible databases for the pathogenic coronavirus SARS-CoV-2. Thus, there is a growing reservoir of diverse genomic sequence information.

The utility of genomic sequence information is limited by the availability of analytic tools. Computational resources required for analysis have lagged behind accumulation of sequence data. For example, treatment and vaccine development studies have often failed to assess genetic diversity of pathogen population leading to failure of clinical trials. There is a need for improved methods and systems for analysis of genomic sequence information, including a need for methods and systems for analysis of large numbers of diverse genomic sequences of a particular organism, sequence, or gene. Improved analytic methods and systems are needed to inform therapeutic development and potentially predict clinical outcome. Additionally, many existing methods for analyzing genomic sequence information require specialized knowledge of sequence databases, operation of sequence analysis software, and/or distillation of data outputs.

### SUMMARY

The present disclosure provides methods and systems for analysis of genomic sequence information. Genomic sequence information, including microbial genomic sequence information, has proliferated in recent years, e.g., in publicly accessible databases. Development of cost-effective, high throughput sequencing instruments and multiplex sequencing protocols have broadened the appeal of genomic analyses, transforming the field of infectious diseases. However, rather than accounting for the breadth of genomic diversity that is available in public databases, comparative genomic analyses are often guided by a small, biased set of fully annotated stock genomes. These stock genomes are often accepted as representative of the breadth of natural or relevant diversity, but in reality represent a minor-fraction of the natural population. This issue of identifying, analyzing, and/or representing natural diversity is particularly acute, for example, with respect to the study of pathogens, where applicability of developed treatments to diverse pathogen isolates is an important component of overall clinical efficacy. Utilization of available sequences from diverse strains has historically required computational skills, and well-curated, up-to-date genomic resources that include genome annotation across diverse lineages (e.g., across pathogen lineages). At least in part because the large available genomic sequences are not fully-assembled in this manner, and/or available genomic sequences (e.g., of diverse strains of a pathogen) are annotated in an inconsistent manner, genomic analyses (e.g., inter-species or intra-species) are complex in practice. As the number of sequenced genomes multiply, the need for analytic and computational tools is an important component of ensuring optimized utilization of these resources.

Methods and systems of the present disclosure, provide, among other things, methods and systems for characterizing sequence conservation among and between input sequences. As is discussed herein, certain methods and systems of the present disclosure include assignment of a similarity or conservation score to a sequence following a multiple sequence comparison based on percent coverage of the alignment between sequences and on the number of variations between sequences.

In certain embodiments, methods and systems of the present disclosure include one or more of the steps described below. For example, in certain embodiments, methods and systems described herein include a first step of selecting the organism (e.g., a pathogen) for which to acquire genomic sequences to use for comparative analysis. Thus, in certain embodiments, the user indicates in a first step information about the genome(s) from which to extract sequences of interest. A second step can include providing sequences, e.g., by acquiring sequence data from a publicly accessible database such as by download from the National Center for Biotechnology Information database (NCBI), and optionally acquiring from the same or a different source sequence annotation and/or feature information. Sequences can also be provided from direct experimental measurement, for example, reads from high-throughput sequencing systems that utilize physical biological samples. Thus, in certain embodiments, sequences can be provided from direct measurement, downloaded from NCBI databases, or both. Sequence and feature files can be automatically downloaded from certain publicly accessible databases such as the NCBI database. A third step can include pairwise comparison of analyzed sequences e.g., by the Basic Local Alignment Search Tool (BLAST). Pair-wise BLAST analysis establishes the level of sequence diversity of each analyzed sequence of interest across all compared sequences. A fourth step can include compiling information related to all pairwise sequence comparisons, e.g., by generating an output table that compiles information related to sequence conservation. An exemplary table can include information about the presence or absence of a particular sequence, level of diversity in a particular sequence locus, nature of variation in a particular sequence locus, and/or genomic coordinates a particular feature in an analyzed sequence. In various embodiments, each sequence analyzed can be assigned a similarity score based on a defined scoring system in which each sequence is categorized according to percent coverage and number of sequence variations. For instance, in certain embodiments, sequences can be categorized and assigned similarity scores according to Table 2. In some embodiments, coding sequences can then be extracted from analyzed sequences and translated to create nucleotide and amino-acid alignments. An optional fifth step can include the generation of visual displays representing compiled sequence conservation information, e.g., in the form of a graph of diversity, phylogenies (e.g., maximum likelihood or parsimony phylogenies), a heatmap, and/or alignment files. In certain examples, genome- and gene-based phylogenies are created using phylogeny software such as the PhyML or QuickTree programs and saved into separated files.

In various embodiments, steps of methods and systems disclosed herein are achieved by use of a computer processor and software. A particular such proprietary software is referenced herein as “Got_Gene”, written in the R programming language. Got_Gene uses BLAST algorithms and R packages to identify, compare, and characterize the diversity of a set of sequences, and can analyze diversity across thousands of sequences.

In various embodiments, a collection of available genomic sequences (subject sequences, e.g., reference sequences) are compared in a pairwise manner to one or more user-selected sequences (query sequence(s)) to identify clinically relevant sequence features. In various embodiments, methods and systems of the present disclosure utilize collections of genomic sequence information that are available in databases, including publicly accessible databases of genomic sequence information. In certain embodiments, the pairwise comparison includes a pairwise comparison of subject and query genetic sequences, e.g., subject and query coding genetic sequences. In certain embodiments, the pairwise comparison includes a pairwise comparison of proteins encoded by subject and query sequences.

In certain embodiments, methods and systems of the present disclosure can be used to identify sequences and sequence characteristics of therapeutic utility. For example, methods and systems of the present disclosure can be used to identify candidate antigens (e.g., pathogen antigens) for development of anti-antigen therapeutics, such as anti-antigen therapeutic antibodies. In some embodiments, methods and systems of the present disclosure can be used to identify candidate vaccine antigens. In some embodiments, methods and systems of the present disclosure can be used to determine whether one or more particular genetic sequences (e.g., the genome of a laboratory pathogen strain) is representative of a collection of comparable genetic sequences (e.g., genomes of a clinically relevant pathogen strains). In some embodiments, methods and systems of the present disclosure can be used to identify antibiotic resistance markers. In some embodiments, methods and systems of the present disclosure can be used to generate peptide discovery resources, e.g., a list of expected peptides and characteristics for use in querying mass spectrometry data. In some embodiments, methods and systems of the present disclosure can be used to identify regions of diversity within sequences. In some embodiments, methods and systems of the present disclosure can be used to generate phylogenies, e.g., to enhance clinical understanding of an epidemic (e.g., the spread of a pathogen). In some embodiments, methods and systems of the present disclosure can be used to identify orthologous sequences between or among species.

A pathogen of the present disclosure can include any pathogen that includes or is characterized by nucleic acid or amino acid sequence(s). Pathogens of the present disclosure included prokaryotic pathogens and eukaryotic pathogens. Examples of pathogens of the present disclosure include, without limitation, bacteria, yeast, protozoa, and viruses. In various embodiments, a pathogen of the present disclosure is selected from Acinetobacter baumannii, Acinetobacter lwoffii, Acinetobacter spp. (e.g., multidrug-resistant Acinetobacter (MDR-A)), Actinomycetes, Adenovirus, Aeromonas spp., Alcaligenes faecalis, Alcaligenes spp./Achromobacter spp., Alcaligenes xylosoxidans (e.g., extended-spectrum beta-lactamase (ESBL)/multidrug-resistant Gram-negative organisms (MRGN)), Arbovirus, Ascaris lumbricoides, Aspergillus spp., Astrovirus, Bacillus anthracis, Bacillus cereus, Bacillus subtilis, Bacteroides fragilis, Bartonella quintana, Blastocystis hominis, Bordetella pertussis, Borrelia burgdorferi, Borrelia duttoni, Borrelia recurrentis, Brevundimonas diminuta, Brevundimonas vesicularis, Brucella spp., Burkholderia cepacia (e.g., multidrug-resistant (MDR)), Burkholderia mallei, Burkholderia pseudomallei, Campylobacter jejuni/coli, Candida albicans, Candida auris, Candida krusei, Candida parapsilosis, Chikungunya virus (CHIKV), Chlamydia pneumoniae, Chlamydia psittaci, Chlamydia trachomatis, Citrobacter spp., Clostridium botulinum, Clostridium difficile, Clostridium perfringens, Clostridium tetani, Coronavirus (e.g., Severe Acute Respiratory Syndrome-associated coronavirus (SARS-CoV); Severe Acute Respiratory Syndrome coronavirus 2 (SARS-CoV2), which is the virus that causes the coronavirus disease (COVID-19); and Middle East Respiratory Syndrome-associated coronavirus (MERS-CoV)), Corynebacterium diphtheriae, Corynebacterium pseudotuberculosis, Corynebacterium spp., Corynebacterium ulcerans, Coxiella burnetii, Coxsackievirus, Crimean-Congo haemorrhagic fever virus, Cryptococcus neoformans, Cryptosporidium hominis, Cryptosporidium parvum, Cyclospora cayetanensis, Cytomegalovirus, Dengue virus, Dientamoeba fragilis, Ebola virus, Echinococcus spp., Echovirus, Entamoeba dispar, Entamoeba histolytica, Enterobacter aerogenes, Enterobacter cloacae (e.g., ESBL/MRGN), Enterobius vermicularis, Enterococcus faecalis (e.g., vancomycin-resistant enterococcus (VRE)), Enterococcus faecium (e.g., VRE), Enterococcus hirae, Epidermophyton spp., Epstein-Barr virus, Escherichia coli (e.g., enterohaemorrhagic E. coli (EHEC), entheropathogenic E. coli (EPEC), enterotoxigenic E coli (ETEC), enteroinvasive E. coli (EIEC), enteroaggregative E. coli (EAEC), ESBL/MRGN, diffusely adhering E. coli (DAEC)), Filarial worms, Foot-and-mouth disease virus (FMDV), Francisella tularensis, Giardia lamblia, Haemophilus influenzae, Hantavirus, Helicobacter pylori, Helminths (Worms), Hepatitis A virus, Hepatitis B virus, Hepatitis C virus, Hepatitis D virus, Hepatitis E virus, Herpes simplex virus, Histoplasma capsulatum, Human T-cell leukemia virus, type 1 (HTLV-1), Human enterovirus 71, Human herpesvirus 6 (HHV-6), Human herpesvirus 7 (HHV-7), Human herpesvirus 8 (HHV-8), Human immunodeficiency virus, Human metapneumovirus, Human papillomavirus, Hymenolepsis nana, Influenza virus (e.g., A(H1N1), A(H1N1)pdm09, A(H3N2), A(H5N1), A(H5N5), A(H5N6), A(H5N8), A(H7N9), A(H10N8)), Klebsiella granulomatis, Klebsiella oxytoca (e.g., ESBL/MRGN), Klebsiella pneumoniae MDR (e.g., ESBL/MRGN), Lassa virus, Leclercia adecarboxylata, Legionella pneumophila, Leishmania spp., Leptospira interrogans, Leuconostoc pseudomesenteroides, Listeria monocytogenes, Marburg virus, Measles virus, Mengla virus, Micrococcus luteus, Microsporum spp., Molluscipoxvirus, Moraxella catarrhalis, Morganella spp., Mumps virus, Mycobacterium basiliense sp. nov., Mycobacterium chimaera, Mycobacterium leprae, Mycobacterium tuberculosis (e.g., MDR), Mycoplasma genitalium, Mycoplasma pneumoniae, Naegleria fowleri, Neisseria meningitidis, Neisseria gonorrhoeae, Nipah virus, Norovirus, Opisthorchis viverrini, Orientia tsutsugamushi, Pantoea agglomerans, Paracoccus yeei, Parainfluenza virus, Parvovirus, Pediculus humanus capitis, Pediculus humanus corporis, Plasmodium spp., Pneumocystis jiroveci, Poliovirus, Polyomavirus, Prevotella spp., Prions, Propionibacterium species, Proteus mirabilis (e.g., ESBL/MRGN), Proteus vulgaris, Providencia rettgeri, Providencia stuartii, Pseudomonas aeruginosa, Pseudomonas spp., Rabies virus, Ralstonia spp., Respiratory syncytial virus, Rhinovirus, Rickettsia prowazekii, Rickettsia typhi, Roseomonas gilardii, Rotavirus, Rubella virus, Schistosoma mansoni, Salmonella enteritidis, Salmonella paratyphi, Salmonella spp., Salmonella typhi, Salmonella typhimurium, Sarcoptes scabiei (Itch mite), Sapovirus, Serratia marcescens (e.g., ESBL/MRGN), Shigella sonnei, Sphingomonas species, Staphylococcus aureus (e.g., methicillin resistant S. aureus MRSA, vancomycin resistant S. aureus (VRSA)), Staphylococcus capitis, Staphylococcus epidermidis (e.g., methicillin-resistant S. epidermidis (MRSE)), Staphylococcus haemolyticus, Staphylococcus hominis, Staphylococcus lugdunensis, Staphylococcus pasteuri, Staphylococcus saprophyticus, Stenotrophomonas maltophilia, Streptococcus pneumoniae, Streptococcus pyogenes (e.g., PRSP), Streptococcus spp., Strongyloides stercoralis, Taenia solium, TBE virus, Toxoplasma gondii, Treponema pallidum, Trichinella spiralis, Trichomonas vaginalis, Trichophyton spp., Trichosporon spp., Trichuris trichiura, Trypanosoma brucei gambiense, Trypanosoma brucei rhodesiense, Trypanosoma cruzi, Usutu virus, Vaccinia virus, Varicella zoster virus, Variola virus, Vibrio cholerae, West Nile virus (WNV), Yellow fever virus, Yersinia enterocolitica, Yersinia pestis, Yersinia pseudotuberculosis, and Zika virus.

In at least one aspect, the present disclosure includes a method for identifying amino acid sequences as candidate antigens in the development of a therapy against a pathogen, comprising: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of the portion among the different strains of the pathogen; selecting portions of the amino acid sequences classified as conserved, comparing the selected conserved sequences to human protein sequences, and further classifying the selected conserved sequences as identical or not identical to a human protein sequence; and categorizing a selected conserved sequence not identical to a human protein sequence as a candidate antigen in the development of a therapy against the pathogen. In various embodiments, extracting can include, for example, identifying, demarcating, or isolating a sequence, e.g., by selecting sequence endpoints. In various embodiments, extracting can include assigning to a sequence or portion of a sequence one or more particular characteristics or statuses, e.g., status as a coding sequence. In various embodiments, extracting can include identifying that a sequence, such as a sequence that has been categorized according to a measure of identity and a measure of coverage, is, in fact, a coding sequence, e.g., by observing annotations (e.g., annotation of a corresponding and/or aligned sequence of a reference as a coding sequence or non-coding sequence, and/or annotation of the genomic position of the categorized sequence). In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, categorizing the selected conserved sequence as a candidate antigen further comprises determining the presence or absence of one or more amino acid domains in the selected conserved sequence. In certain embodiments, categorizing the selected conserved sequence as a candidate antigen further comprises determining whether the candidate antigen corresponds to a protein that is secreted or is exposed within a membrane and/or cell wall of the pathogen. In certain embodiments, categorizing the selected conserved sequence as a candidate antigen further comprises determining the presence of a transmembrane domain in a selected conserved sequence. In certain embodiments, the therapy comprises a vaccine and the method further comprises non-clinically evaluating the candidate antigen for immunogenicity. In certain embodiments, the evaluating step comprises administering a polypeptide comprising the candidate antigen to an animal, e.g., where the animal is a human, non-human primate, mouse, or rat. In certain embodiments, the therapy comprises an antibody therapy, and the method further comprises producing an antibody or fragment thereof that specifically binds to an epitope on the candidate antigen. In certain embodiments, the pathogen is a virus. In certain embodiments, the virus is methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the pathogen is a bacterium. In certain embodiments, the bacterium is a Staphylococcus species or a Pseudomonas species. In certain embodiments, the method includes producing a therapeutic agent that targets or binds the candidate antigen. In certain embodiments, the therapeutic agent is an antibody or inhibitor. In certain embodiments, the therapeutic agent is an shRNA or siRNA that corresponds to a nucleic acid sequence such as a coding sequence that encodes the candidate antigen.

In at least one aspect, the present disclosure includes a method of identifying one or more putative escape mutations after administration of a therapeutic agent to one or more subjects for treatment of a pathogen infection, comprising: obtaining a plurality of complete or partial pathogen genomic sequences isolated from one or more subjects after administration of the therapeutic agent to each subject; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; identifying, in the aligned amino acid sequences, one or more amino acid variants more frequent in the aligned amino acid sequences than in a reference, the one or more amino acid variants being one or more putative escape mutations. In certain embodiments, the reference comprises one or more complete or partial pathogen genomic sequences representative of a canonical pathogen sequence, one or more clinical strains of the pathogen, one or more earlier samples of pathogen from one or more of the subjects administered the therapeutic agent, or one or more samples of pathogen from subjects not administered the therapeutic agent. In certain embodiments, the method further comprises a step of determining whether one or more of the putative escape mutations decreases binding affinity of the therapeutic agent with a reference polypeptide. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the therapeutic agent is an antibody or inhibitor. In certain embodiments, the therapeutic agent is an shRNA or siRNA. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the therapeutic agent comprises a therapeutic agent that treats COVID-19. In certain embodiments, the therapeutic agent comprises remdesivir, kaletra, ivermectin, tamiflu, avigan, colcrys, dexamethasone, chloroquine, hydroxychloroquine, azithromycin, it-6 inhibitors (e.g., tocilizumab and sarilumab), kinase inhibitors (e.g., acalabrutinib, ibrutinib, zanubrutinib, baricitinib, ruxolitinib, and tofacitinib), interferons, convalescent plasma, antibodies that bind SARS-CoV-2 spike protein (anti-SARS-CoV-2-Spike protein antibodies), mAb10933 (Regeneron), mAb10934 (Regeneron), mAb10987 (Regeneron), mAb10989 (Regeneron), REGN-COV2 (Regeneron), LY-CoV555 (Eli Lilly), LY-CoV016 (Eli Lilly), and/or BNT162b2 (Pfizer). In certain embodiments, the pathogen is a bacterium. In certain embodiments, the bacterium is a Staphylococcus species or a Pseudomonas species. In certain embodiments, the method includes, after identifying one or more putative escape mutations, administering to the one or more subjects a different therapeutic agent. In certain embodiments, the different therapeutic agent comprises a therapeutic agent that treats COVID-19. In certain embodiments, the different therapeutic agent comprises remdesivir, kaletra, ivermectin, tamiflu, avigan, colcrys, dexamethasone, chloroquine, hydroxychloroquine, azithromycin, it-6 inhibitors (e.g., tocilizumab and sarilumab), kinase inhibitors (e.g., acalabrutinib, ibrutinib, zanubrutinib, baricitinib, ruxolitinib, and tofacitinib), interferons, convalescent plasma, antibodies that bind SARS-CoV-2 spike protein (anti-SARS-CoV-2-Spike protein antibodies), mAb10933 (Regeneron), mAb10934 (Regeneron), mAb10987 (Regeneron), mAb10989 (Regeneron), REGN-COV2 (Regeneron), LY-CoV555 (Eli Lilly), LY-CoV016 (Eli Lilly), and/or BNT162b2 (Pfizer).

In at least one aspect, the present disclosure includes a method of administering a therapeutic agent for treatment of a pathogen infection to a subject in need thereof, comprising: selecting a conserved portion of an amino acid sequence by: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of the portion among the different strains of the pathogen; and selecting a conserved portion of the aligned amino acid sequences; and administering the therapeutic agent to a subject if a complete or partial pathogen genomic sequence isolated from the subject encodes the conserved portion of an amino acid sequence, where the therapeutic agent selectively binds the conserved portion of the amino acid sequence. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the therapeutic agent comprises a therapeutic agent that treats COVID-19. In certain embodiments, the therapeutic agent comprises remdesivir, kaletra, ivermectin, tamiflu, avigan, colcrys, dexamethasone, chloroquine, hydroxychloroquine, azithromycin, it-6 inhibitors (e.g., tocilizumab and sarilumab), kinase inhibitors (e.g., acalabrutinib, ibrutinib, zanubrutinib, baricitinib, ruxolitinib, and tofacitinib), interferons, convalescent plasma, antibodies that bind SARS-CoV-2 spike protein (anti-SARS-CoV-2-Spike protein antibodies), mAb10933 (Regeneron), mAb10934 (Regeneron), mAb10987 (Regeneron), mAb10989 (Regeneron), REGN-COV2 (Regeneron), LY-CoV555 (Eli Lilly), LY-CoV016 (Eli Lilly), and/or BNT162b2 (Pfizer). In certain embodiments, the pathogen is a bacterium. In certain embodiments, the bacterium is a Staphylococcus species or a Pseudomonas species.

In at least one aspect, the present disclosure includes a method for selecting a therapeutic agent for treatment of subjects infected with a pathogen, comprising: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of the portion among the different strains of the pathogen, thereby identifying a conserved portion of a coding sequence representative of the pathogen; and selecting a therapeutic agent that binds the conserved coding sequence as a treatment for subjects infected with the pathogen. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the method further comprises non-clinically evaluating the therapeutic agent as a vaccine or component thereof. In certain embodiments, the evaluating step comprises administering the therapeutic agent to an animal, e.g., where the animal is a human, non-human primate, mouse, or rat. In certain embodiments, the method further includes administering the therapeutic agent to a subject infected with the pathogen In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the therapeutic agent comprises a therapeutic agent that treats COVID-19. In certain embodiments, the therapeutic agent comprises remdesivir, kaletra, ivermectin, tamiflu, avigan, colcrys, dexamethasone, chloroquine, hydroxychloroquine, azithromycin, it-6 inhibitors (e.g., tocilizumab and sarilumab), kinase inhibitors (e.g., acalabrutinib, ibrutinib, zanubrutinib, baricitinib, ruxolitinib, and tofacitinib), interferons, convalescent plasma, antibodies that bind SARS-CoV-2 spike protein (anti-SARS-CoV-2-Spike protein antibodies), mAb10933 (Regeneron), mAb10934 (Regeneron), mAb10987 (Regeneron), mAb10989 (Regeneron), REGN-COV2 (Regeneron), LY-CoV555 (Eli Lilly), LY-CoV016 (Eli Lilly), and/or BNT162b2 (Pfizer). In certain embodiments, the pathogen is a bacterium. In certain embodiments, the bacterium is a Staphylococcus species or a Pseudomonas species.

In at least one aspect, the present disclosure includes a method for assessing conservation of portions of amino acid sequences representative of a pathogen, comprising: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; and identifying a level of conservation of one or more portions of amino acid sequences representative of the pathogen using the aligned amino acid sequences. In certain embodiments, one or more of the portions is identified as a candidate antigen in the development of a therapy against the pathogen. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the genomic sequences are SARS-CoV-2 genomic sequences and the reference sequence is a SARS-CoV-2 reference sequence. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the pathogen is a bacterium. In certain embodiments, the bacterium is a Staphylococcus species or a Pseudomonas species.

In at least one aspect, the present disclosure includes a method for identifying whether an isolated pathogen is representative of a circulating strain, comprising: obtaining a plurality of complete or partial genomic sequences of the circulating strain of the pathogen from a data structure; identifying one or more conserved portions of the sequences of the circulating strain; obtaining a plurality of complete or partial genomic sequences of the isolated pathogen; and identifying whether the isolated pathogen is representative of the circulating strain by comparing at least a portion of the sequences of the isolated pathogen against the identified one or more conserved portions of the sequences of the circulating strain. In certain embodiments, identifying one or more conserved portions of the sequences of the circulating strain comprises: extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; and classifying each of a plurality of portions of the aligned amino acid sequences according to a level of conservation of the portion among the aligned amino acid sequences. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the pathogen is a bacterium. In certain embodiments, the bacterium is a Staphylococcus species or a Pseudomonas species. In certain embodiments, the method further includes storing (e.g., freezing) a sample of the isolated pathogen and/or the circulating strain. In certain embodiments, the method further includes isolating genomic material from the isolated pathogen and/or circulating strain and/or storing (e.g., freezing) genomic material isolated from the pathogen and/or circulating strain. In certain embodiments, the method further includes, if the isolated pathogen is representative of the circulating strain, utilizing and/or maintaining the isolated pathogen as a strain for research (e.g., research for development of a therapeutic agent for treatment of the pathogen, optionally where the therapeutic agent can be, for example, an shRNA, siRNA, inhibitor, or antibody).

In at least one aspect, the present disclosure includes a method for identifying a mass-to-charge ratio of a peptide representative of a pathogen, comprising: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; and determining the mass-to-charge ratio of one or more of the amino acid sequences or portions thereof. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the method comprises evaluating a coronavirus spike (S) protein [e.g., MERS, SARS-CoV, or SARS-CoV2 spike (S) protein] or a receptor-binding domain (RBD) thereof. In certain embodiments, the pathogen is a bacterium. In certain embodiments, the bacterium is a Staphylococcus species or a Pseudomonas species. In certain embodiments, the method further includes performing mass spectrometry of one or more polypeptides from a sample of the pathogen and/or determining whether the polypeptides from the sample are or include amino acid sequences that have mass-to-charge ratios matching the determined mass-to-charge ratios.

In at least one aspect, the present disclosure includes a method for identifying an amino acid sequence as a candidate antibiotic resistance marker, comprising: obtaining a plurality of complete or partial plasmid sequences of a pathogenic bacterium from a data structure; extracting, by a processor of a computing device, coding sequences from the plasmid sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of the portion among the plurality of plasmid sequences; selecting portions of the amino acid sequences classified as conserved; and categorizing a selected conserved sequence as a candidate antibiotic resistance marker. In certain embodiments, the method further comprises identifying the candidate antibiotic resistance marker as a candidate according to one or more additional criteria comprising a presence of a transmembrane domain in a selected sequence. In certain embodiments, the data structure comprises contigs, and where obtaining the plurality of complete or partial plasmid sequences from the data structure comprises merging, by the processor, overlapping contigs to produce at least some of the complete or partial plasmid sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the bacterium is a Staphylococcus species or a Pseudomonas species. In certain embodiments, the method further includes screening one or more samples from one or more subjects for presence or absence of the candidate antibiotic resistance marker, e.g., where the one or more subjects are infected with the pathogenic bacterium.

In at least one aspect, the present disclosure includes a method for identifying one or more conserved portions of coding sequences representative of a plasmid, comprising: obtaining a plurality of complete or partial plasmid sequences of a pathogenic bacterium from a data structure; extracting, by a processor of a computing device, coding sequences from the plasmid sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; and classifying each of a plurality of portions of the amino acid sequences according to a level of conservation of the portion among the plurality of plasmid sequences, thereby identifying one or more conserved portions of coding sequences representative of the plasmid. In certain embodiments, the data structure comprises contigs, and where obtaining the plurality of complete or partial plasmid sequences from the data structure comprises merging, by the processor, overlapping contigs to produce at least some of the complete or partial plasmid sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the method comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the bacterium is a Staphylococcus species or a Pseudomonas species. In certain embodiments, the method further includes screening one or more samples from one or more subjects for presence or absence of the conserved portions of coding sequences representative of the plasmid, e.g., where the one or more subjects are infected with the pathogenic bacterium.

In at least one aspect, the present disclosure includes a system for automatically identifying one or more conserved portions of coding sequences representative of a pathogen, the system comprising: a processor; and a memory having instructions thereon, the instructions, when executed by the processor, causing the processor to: obtain a plurality of complete or partial genomic sequences of different strains of the pathogen from a data structure; extract, by the processor, coding sequences from the genomic sequences; categorize, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; select coding sequences from among the categorized coding sequences according to the measure of identity and the measure of coverage; convert, by the processor, the selected coding sequences into corresponding amino acid sequences; align, by the processor, the amino acid sequences; and classify each of a plurality of portions of the aligned amino acid sequences according to a level of conservation of the portion among the different strains of the pathogen, thereby identifying one or more conserved portions of coding sequences representative of the pathogen. In certain embodiments, the instructions, when executed by the processor, cause the processor to compute, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the instructions, when executed by the processor, cause the processor to create a matrix of the measures of similarity and render a graphical representation of the matrix, thereby displaying levels of conservation between the query sequences and subject sequences. In certain embodiments, the graphical representation comprises one or more of a heatmap, a graph, and a phylogeny. In certain embodiments, the data structure comprises contigs, and where the instructions, when executed by the processor, cause the processor to obtain the plurality of complete or partial genomic sequences of different strains of the pathogen by merging, by the processor, overlapping contigs to produce at least some of the complete or partial genomic sequences. In certain embodiments, the instructions, when executed by the processor, cause the processor to evaluate one or more of: coding sequences of a nucleic acid that encodes a protein associated with the pathogen; conserved sequences of a nucleic acid sequence that encodes a protein associated with the pathogen non-conserved sequences of a nucleic acid that encodes a protein; conserved domains within a particular protein associated with the pathogen; and non-conserved domains within a particular protein associated with the pathogen. In certain embodiments, the instructions, when executed by the processor, cause the processor to evaluate a coronavirus spike (S) protein [e.g., MERS, SARS-CoV, or SARS-CoV2 spike (S) protein] or a receptor-binding domain (RBD) thereof. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the pathogen is a bacterium. In certain embodiments, the bacterium is a Staphylococcus species or a Pseudomonas species.

In at least one aspect, the present disclosure includes a system for automatically identifying one or more conserved portions of coding sequences representative of a plasmid, the system comprising: a processor; and a memory having instructions thereon, the instructions, when executed by the processor, causing the processor to: obtain a plurality of complete or partial plasmid sequences of a pathogenic bacterium from a data structure; extract, by the processor, coding sequences from the plasmid sequences; categorize, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; select coding sequences from among the categorized coding sequences according to the measure of identity and the measure of coverage; convert, by the processor, the selected coding sequences into corresponding amino acid sequences; align, by the processor, the amino acid sequences; and classify each of a plurality of portions of the amino acid sequences according to a level of conservation of the portion among the plurality of plasmid sequences, thereby identifying one or more conserved portions of coding sequences representative of the plasmid. In certain embodiments, the instructions, when executed by the processor, cause the processor to compute, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the instructions, when executed by the processor, cause the processor to create a matrix of the measures of similarity and render a graphical representation of the matrix, thereby displaying levels of conservation between the query sequences and subject sequences. In certain embodiments, the graphical representation comprises one or more of a heatmap, a graph, and a phylogeny. In certain embodiments, the data structure comprises contigs, and where the instructions, when executed by the processor, cause the processor to obtain the plurality of complete or partial plasmid sequences of a pathogenic bacterium by merging, by the processor, overlapping contigs to produce at least some of the complete or partial plasmid sequences. In certain embodiments, the instructions, when executed by the processor, cause the processor to evaluate one or more of: coding sequences of a nucleic acid that encodes a protein associated with the pathogen; conserved sequences of a nucleic acid sequence that encodes a protein associated with the pathogen non-conserved sequences of a nucleic acid that encodes a protein; conserved domains within a particular protein associated with the pathogen; and non-conserved domains within a particular protein associated with the pathogen. In certain embodiments, the instructions, when executed by the processor, cause the processor to evaluate a coronavirus spike (S) protein [e.g., MERS, SARS-CoV, or SARS-CoV2 spike (S) protein] or a receptor-binding domain (RBD) thereof. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the pathogen is a bacterium. In certain embodiments, the bacterium is a Staphylococcus species or a Pseudomonas species.

In at least one aspect, the present disclosure includes a therapeutic agent for use in identifying one or more putative escape mutations after administration of the therapeutic agent to one or more subjects for treatment of a pathogen infection, the use comprising: obtaining a plurality of complete or partial pathogen genomic sequences isolated from one or more subjects after administration of the therapeutic agent to each subject; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; identifying, in the aligned amino acid sequences, one or more amino acid variants more frequent in the aligned amino acid sequences than in a reference, the one or more amino acid variants being one or more putative escape mutations. In certain embodiments, the reference comprises one or more complete or partial pathogen genomic sequences representative of a canonical pathogen sequence, one or more clinical strains of the pathogen, one or more earlier samples of pathogen from one or more of the subjects administered the therapeutic agent, or one or more samples of pathogen from subjects not administered the therapeutic agent. In certain embodiments, the use further comprises a step of determining whether one or more of the putative escape mutations decreases binding affinity of the therapeutic agent with a reference polypeptide. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the use comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the use comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the pathogen is a bacterium. In certain embodiments, the bacterium is a Staphylococcus species or a Pseudomonas species.

In at least one aspect, the present disclosure includes a therapeutic agent for use in treatment of a pathogen infection, the use comprising: selecting a conserved portion of an amino acid sequence by: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of the portion among the different strains of the pathogen; and selecting a conserved portion of the aligned amino acid sequences; and administering the therapeutic agent to a subject if a complete or partial pathogen genomic sequence isolated from the subject encodes the conserved portion of an amino acid sequence, where the therapeutic agent selectively binds the conserved portion of the amino acid sequence. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the use comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the pathogen is a bacterium. In certain embodiments, the bacterium is a Staphylococcus species or a Pseudomonas species.

In at least one aspect, the present disclosure includes use of a therapeutic agent for the manufacture of a medicament for identifying one or more putative escape mutations after administration of the medicament to one or more subjects for treatment of a pathogen infection, the use including: obtaining a plurality of complete or partial pathogen genomic sequences isolated from one or more subjects after administration of the medicament to each subject; extracting, by a processor of a computing device, coding sequences from the genomic sequences; categorizing, by the processor, the coding sequences according to a measure of identity and a measure of coverage, where the measure of identity includes one or more of percent identity, percent identity over a predetermined coverage length, number of mutations, and percent mutation, and where the measure of coverage includes one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; identifying, in the aligned amino acid sequences, one or more amino acid variants more frequent in the aligned amino acid sequences than in a reference, said one or more amino acid variants being one or more putative escape mutations. In certain embodiments, the reference comprises one or more complete or partial pathogen genomic sequences representative of a canonical pathogen sequence, one or more clinical strains of the pathogen, one or more earlier samples of pathogen from one or more of the subjects administered the therapeutic agent, or one or more samples of pathogen from subjects not administered the therapeutic agent. In certain embodiments, the use further comprises a step of determining whether one or more of the putative escape mutations decreases binding affinity of the therapeutic agent with a reference polypeptide. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, the use comprises evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the use comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the pathogen is a bacterium. In certain embodiments, the bacterium is a Staphylococcus species or a Pseudomonas species.

In at least one aspect, the present disclosure includes use of a therapeutic agent for the manufacture of a medicament for treatment of a pathogen infection, the use including: selecting a conserved portion of an amino acid sequence by: obtaining a plurality of complete or partial genomic sequences of different strains of the pathogen from a data structure; extracting, by a processor of a computing device, coding sequences from the genomic sequences; categorizing, by the processor, the coding sequences according to a measure of identity and a measure of coverage, where the measure of identity includes one or more of percent identity, percent identity over a predetermined coverage length, number of mutations, and percent mutation, and where the measure of coverage includes one or more of percent coverage and coverage length; selecting coding sequences from among the categorized coding sequences according to the measure of identity and the measure of coverage; converting, by the processor, the selected coding sequences into corresponding amino acid sequences; aligning, by the processor, the amino acid sequences; classifying each of a plurality of portions of the aligned amino acid sequences according to a level of conservation of said portion among the different strains of the pathogen; and selecting a conserved portion of the aligned amino acid sequences; and administering the medicament to a subject if a complete or partial pathogen genomic sequence isolated from the subject encodes the conserved portion of an amino acid sequence, where the therapeutic agent selectively binds the conserved portion of the amino acid sequence. In certain embodiments, the data structure comprises contigs, and where obtaining the plurality of complete or partial genomic sequences of different strains of the pathogen from the data structure comprises merging, by the processor, overlapping contigs to produce at least a portion of the complete or partial genomic sequences. In certain embodiments, the categorizing step comprises quantifying the measure of identity and the measure of coverage for each of a plurality of pairs, each of the pairs comprising an extracted coding sequence and a reference sequence. In certain embodiments, the categorizing step comprises computing, for each of a set of query coding sequences against a set of subject sequences, measures of similarity between the query coding sequence and each subject sequence, each of the measures of similarity a function of a measure of identity between the query sequence and the subject sequence and a measure of coverage between the query sequence and the subject sequence. In certain embodiments, the computing step comprises creating a matrix of the measures of similarity and rendering a graphical representation of the matrix, thereby displaying levels of conservation between the query sequences and subject sequences. In certain embodiments, the graphical representation comprises one or more of a heatmap, a graph, and a phylogeny. In certain embodiments, the measure of identity comprises number of mutations. In certain embodiments, the measure of coverage comprises percent coverage. In certain embodiments, the measure of identity comprises calculating E-value. In certain embodiments, evaluating one or more of: coding sequences of a nucleic acid that encodes a protein associated with the pathogen; conserved sequences of a nucleic acid sequence that encodes a protein associated with the pathogen; non-conserved sequences of a nucleic acid that encodes a protein; conserved domains within a particular protein associated with the pathogen; and non-conserved domains within a particular protein associated with the pathogen. In certain embodiments, each portion of an amino acid sequence comprises one or more amino acid positions. In certain embodiments, the pathogen is a virus. In certain embodiments, the virus is Methicillin-resistant Staphylococcus aureus (MRSA), Hepatitis B Virus (HBV), influenza, or Ebola virus. In certain embodiments, the virus is a coronavirus. In certain embodiments, the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV). In certain embodiments, the coronavirus is SARS-CoV-2. In certain embodiments, the use comprises evaluating a coronavirus spike (S) protein [e.g., MERS, SARS-CoV, or SARS-CoV2 spike (S) protein] or a receptor-binding domain (RBD) thereof. In certain embodiments, the therapeutic agent comprises an antibody. In certain embodiments, the antibody binds SARS-CoV-2. In certain embodiments, the antibody binds SARS-CoV-2 spike protein. In certain embodiments, the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3. In certain embodiments, the pathogen is a bacterium. In certain embodiments, the bacterium is a Staphylococcus species or a Pseudomonas species.

In at least one aspect, the present disclosure includes a method of determining whether a pathogen epitope bound by an antibody is conserved, comprising: obtaining a plurality of complete or partial genomic sequences of different strains of the pathogen from a data structure; extracting, by a processor of a computing device, coding sequences from the genomic sequences; comparing the coding sequences to a reference sequence encoding the pathogen epitope; categorizing, by the processor, the coding sequences according to a measure of identity and a measure of coverage, where the measure of identity comprises one or more of percent identity, percent identity over a predetermined coverage length, number of mutations, and percent mutation, and where the measure of coverage comprises one or more of percent coverage and coverage length; selecting coding sequences from among the categorized coding sequences according to the measure of identity and the measure of coverage; converting the selected coding sequences into corresponding amino acid sequences; and determining the level of conservation of the pathogen epitope among the different strains of the pathogen.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The Drawings included herein, which are composed of the following Figures, are for illustrative purposes only and not for limitation.

FIG. 1 is a schematic that shows an exemplary sequence analysis workflow, according to an illustrative embodiment.

FIG. 2 is a schematic that shows an exemplary set of information to be provided when extracting sequences from publicly accessible databases, or when manually providing sequences, for analysis according to a method or system of the present disclosure.

FIG. 3 is a schematic that shows an exemplary system of organizing data into folders for analysis according to a method or system of the present disclosure.

FIG. 4 is a schematic that shows an exemplary distribution of copies of sequences and/or annotation information downloaded from one or more publicly accessible databases (e.g., NCBI) into folders, according to an illustrative embodiment. As shown in FIG. 4, downloaded sequences and/or annotation information is copied into three folders: Reference Sequences, Aligner Databases, and Annotation Folder.

FIG. 5 is a schematic that shows exemplary steps for downloading and curating sequences from an exemplary publicly accessible database (NCBI), according to an illustrative embodiment.

FIG. 6 is a schematic that shows exemplary steps for entering query sequences for use in a method or system of the present disclosure.

FIG. 7 is a schematic that shows an exemplary approach to pairwise BLAST comparison of query sequences and subject sequences (reference sequences) stored in a Query Sequences folder and an Aligner Databases folder, respectively, according to an illustrative embodiment.

FIG. 8 is a schematic that shows exemplary steps for application of BLAST to perform pairwise sequence comparisons of query sequences and subject sequences (reference sequences), according to an illustrative embodiment.

FIG. 9 is a schematic that shows an exemplary compilation of BLAST results, sequence information, and sequence annotation information to generate a Gene Output Table (“Got Table”), according to an illustrative embodiment.

FIG. 10 is a schematic that shows exemplary steps for compiling BLAST results for inclusion in a Got Table, according to an illustrative embodiment.

FIG. 11 is a schematic that shows exemplary steps for compiling information related to contigs in a Got Table, according to an illustrative embodiment.

FIG. 12 is a schematic that shows exemplary steps for identifying matched sequences after pairwise comparison, calculating the percent mutation of matched sequences, and compiling feature file annotations available in the publicly accessible database (NCBI), according to an illustrative embodiment.

FIG. 13 is a schematic that shows exemplary content of a Got Table, according to an illustrative embodiment.

FIG. 14 is a schematic that shows exemplary steps for generating a Comparative Table for each query sequence including a matrix of similarity scores for pairwise comparisons, which similarity scores values assigned based on percent coverage and number of mutations, according to an illustrative embodiment.

FIG. 15 is a schematic that shows exemplary steps for representing similarity scores in a heatmap or in a bar plot, according to an illustrative embodiment.

FIG. 16 is a schematic that shows exemplary steps for extracting coding sequences, which extracted sequences can be translated and aligned, according to an illustrative embodiment. Steps provide an exemplary approach to contigs. Steps provide an exemplary approach to generating a table that includes the number and frequency of unique versions of an extracted sequence.

FIG. 17 is a schematic that shows an exemplary approach for creation of phylogenies from extracted coding sequences, according to an illustrative embodiment.

FIG. 18 is a schematic that shows exemplary steps for production of a Got Table and exemplary out puts that can be generated from data present in a Got Table, according to an illustrative embodiment.

FIG. 19 is a graph that shows exemplary bacterial genomes represented in NCBI and suitable for use in an analysis according to methods and systems disclosed herein.

FIG. 20 is a schematic that shows an exemplary system as disclosed herein.

FIG. 21 is a schematic that represents infection of a human with Hepatitis B Virus (HBV) which infection can lead to hepatocellular carcinoma.

FIG. 22 is a schematic that shows an exemplary HBV circular genome.

FIG. 23 is a schematic that shows an exemplary HVC circular genome with the gene S identified by a bracket.

FIG. 24 is a schematic that shows an exemplary distribution of genotypes of HBV.

FIG. 25 is a schematic that shows exemplary sequence structures suitable for analysis according to methods and systems of the present disclosure, including circular, linear, and fragmented sequences that are provided manually and/or downloaded from a publicly accessible database such as NCBI.

FIG. 26 is a schematic that represents extraction of coding sequences from a genomic sequence, according to an illustrative embodiment. Extracted coding sequences from a genomic sequence can be found in the genomic sequence in various lengths and orientations.

FIG. 27 is a schematic that represents an exemplary pairwise BLAST comparison of a single coding sequence from a collection of query coding sequences with each of a plurality of input genomic sequences, e.g., comparison of an extracted query coding sequence from a collection of extracted query coding sequences with each of a plurality of subject sequences that are reference genomic sequences, according to an illustrative embodiment. At least in part because subject sequences such as reference sequences can vary in nucleotide sequence and content, alignment of an extracted query sequence with each reference sequence can vary in relative position of alignment, coverage length, and/or orientation. In some embodiments, a subject sequence and a reference sequence will not be found to have corresponding sequences (i.e., comparison may produce “no hits” in one more particular subject genomic sequences). In certain embodiments, coding sequences are extracted from subject genomic sequences, each subject coding sequence is compared (e.g., by BLAST) with one or more query genomic sequences, and one or more sequence categorization factors (e.g., coverage length and percent identity) are determined for each comparison. In various embodiments, if coverage length and percent identity are each greater than a respective threshold value, a corresponding query sequence is extracted and can be further analyzed or evaluated. The threshold values are applied to determine whether each query genomic sequence or portion thereof is similar to a reference sequence. Methods and systems provided herein are applicable to genomic sequences that represent complete genomes as well as genomic sequences that represent one or more portions of a complete genome.

FIG. 28 is a schematic that shows an exemplary summary of results of pairwise BLAST comparison of a single reference sequence with each of a plurality of input query genomic sequences, e.g., comparison of a plurality of query coding sequence with a subject genomic sequences that is a reference genomic sequence, according to an illustrative embodiment. Column 1 of the summary indicates a reference genomic sequence (B Lee 1940) to which query genomic sequences were compared. In particular, the shown table relates to a particular gene of the reference genomic sequence encoding a particular known product annotated in the reference genomic sequence, hemagglutinin. The table shows that the hemagglutinin reference sequence from the reference genome was compared to each of 9 query genomes. Categorization factors were used to determine whether the a sequence corresponding to hemagglutinin was present in each query genome (yes, no, or partially, as indicated in the “gene presence” column). The orientation (“strand”) of the corresponding query sequence was also included in the table. For each comparison, percent coverage, number of mutations (SNPs), and alignment gaps were noted in the table.

FIG. 29 is a schematic that shows four exemplary plots each showing the number of subject genomes with specified numbers and types of variations as compared to one of four query sequences, according to an illustrative embodiment.

FIG. 30 is a schematic that shows an exemplary heatmap of similarity scores representing level of conservation between each of 20 exemplary subject sequences that are reference genomic sequences (X axis) and each of eight exemplary query coding sequences, according to an illustrative embodiment.

FIG. 31 is an exemplary presentation of a whole genome phylogeny for FluA contemporary strains, according to an illustrative embodiment.

FIG. 32 is a schematic that shows exemplary phylogeny in rectangular layout, according to an illustrative embodiment.

FIG. 33 is a schematic that shows an exemplary phylogeny in polar layout, according to an illustrative embodiment.

FIG. 34 is a schematic that shows exemplary coding sequences extracted from genomic sequences, according to an illustrative embodiment.

FIG. 35 is a schematic that shows translations of the exemplary coding sequences of FIG. 34, and includes a summary of particular variant sequences and their frequencies within analyzed genomes, according to an illustrative embodiment.

FIG. 36 is a schematic that shows an exemplary alignment of amino acid sequences derived from 8 distinct pairwise-compared genomes, according to an illustrative embodiment.

FIG. 37 is a schematic of a computer network environment for use in providing systems and methods described herein.

FIG. 38 is a schematic of a computing device and a mobile computing device that can be used to implement systems and methods described herein.

FIG. 39 is a block flow diagram of an exemplary method for identifying amino acid sequences as candidate antigens in the development of a therapy against a pathogen, according to an illustrative embodiment.

FIG. 40 is a block flow diagram of an exemplary method for identifying one or more conserved portions of coding sequences representative of a pathogen, according to an illustrative embodiment.

FIG. 41 is a block flow diagram of an exemplary method for identifying whether an isolated pathogen is representative of a circulating strain, according to an illustrative embodiment.

FIG. 42 is a block flow diagram of an exemplary method for identifying an amino acid sequence as a candidate antibiotic resistance marker, according to an illustrative embodiment.

FIG. 43 is a block flow diagram of an exemplary method for identifying one or more conserved portions of coding sequences representative of a plasmid, according to an illustrative embodiment.

FIG. 44 is a block flow diagram of an exemplary method for identifying a mass-to-charge ratio of a peptide representative of a pathogen, for example, to identify mass spectrometry targets for such pathogen-representative peptides, according to an illustrative embodiment.

FIG. 45 is a block flow diagram of an exemplary method for identifying amino acid sequences as candidate antigens in the development of a therapy against a pathogen, according to an illustrative embodiment.

FIG. 46 is a block flow diagram of an exemplary method for identifying an amino acid sequence as a candidate antibiotic resistance marker, according to an illustrative embodiment.

FIG. 47 is a schematic of an exemplary coronavirus such as SARS-CoV-2. The coronavirus structure has an exterior lipid membrane, which includes embedded transmembrane proteins including, but not limited to, spike proteins, envelope proteins, and membrane glycoproteins. The schematic includes a representation of a coronavirus RNA viral genome associated with nucleocapsid proteins.

FIG. 48 is a schematic representation of a method of determining amino acid conservation of subject sequences in a set of query sequences. Coding sequences are extracted from query and subject sequences. Pairwise BLAST comparison of extracted query coding sequences and extracted subject coding sequences is performed. Data from pairwise BLAST is used to produce a table of data including categorization factors such as percent identity, percent coverage, coverage length, percent identity over a predetermined coverage length, E-value, number of mutations, and percent mutation for each pairwise comparison. BLAST comparison results are then categorized based on threshold values of one or more categorization factors. Comparisons in categories that do not meet inclusion threshold, and/or meet an exclusion threshold, are removed from analysis. Remaining query sequences are translated and resulting amino acid sequences are aligned with corresponding translated subject sequences. Amino acid conservation of translated subject sequences among the translated query sequences is evaluated from these alignments.

FIG. 49 is a schematic that illustrates extraction of a spike coding sequence from a reference genome. Extraction was based on GenBank file annotations.

FIG. 50 is a graph showing the cumulative number of spike coding sequences compared by BLAST with the reference spike coding sequence over time. As shown by the dates and number of sequences sampled, a large number of sequences were acquired and analyzed, representing sequences isolated in Europe, North America, Asia, Oceania, South America, and Africa.

FIG. 51 is a schematic that illustrates alignment of spike amino acid sequences. Coding sequences retained for analysis after filtering based on number of mutations and coverage length were translated and aligned by BLAST. The aligned sequences can then be inspected and/or compared to identify the range of amino acids present at each aligned position of the reference spike protein sequence.

FIG. 52 is a schematic that illustrates, in part, amino acid variation identified by alignment of amino acid translations of analyzed coding sequences.

### DETAILED DESCRIPTION

Genomic and Plasmid Sequence Information

Methods and systems of the present disclosure include analysis of genomic sequences and/or plasmid sequences. Genomic sequences can include complete and/or partial genomic sequences. Plasmid sequences can include complete and/or partial plasmid sequences. The size and structure of genomes differ among organisms. For instance, eukaryotic genomes typically include a plurality of chromosomes, and prokaryotic genomes typically include a single circular nucleic acid. Prokaryotes can additionally include smaller independent molecules known in the art as plasmids. Plasmids can encode genes, e.g., genes that encode proteins that confer antibiotic resistance (antibiotic resistance markers). Various embodiments disclosed herein as applicable to one form of genetic sequence information are applicable to other forms as well, e.g., that embodiments disclosed in relation to genomic sequences will be applicable to plasmid sequences as well.

A complete genomic sequence can include a single sequence representing the entire genome of an organism. A complete genomic sequence can include a plurality of sequences that together represent the entire genome of an organism. A partial genomic sequence can refer to any single sequence representing a contiguous subset of the nucleic acids of a genomic sequence. A partial genomic sequence can include a plurality of sequences that together represent a contiguous subset of the nucleic acids of a genomic sequence.

In various embodiments, a genomic sequence is a complete or partial sequence of a pathogen genome, e.g., a complete or partial genome of any pathogenic bacteria, yeast, protozoa, or virus. For example, in some embodiments, a genomic sequence is a complete or partial sequence of the genome of a coronavirus, e.g., Severe Acute Respiratory Syndrome-associated coronavirus (SARS-CoV), Severe Acute Respiratory Syndrome coronavirus 2 (SARS-CoV2), or Middle East Respiratory Syndrome-associated coronavirus (MERS-CoV).

A complete plasmid sequence can include a single sequence representing the entire genome of an organism. A complete plasmid sequence can include a plurality of sequences that together represent the entire genome of an organism. A partial plasmid sequence can refer to any single sequence representing a contiguous subset of the nucleic acids of a plasmid sequence. A partial plasmid sequence can include a plurality of sequences that together represent a contiguous subset of the nucleic acids of a plasmid sequence.

In some embodiments, individual sequences that together represent a larger nucleic acid sequence can be referred to as contigs. In some embodiments, contigs can be assembled to provide the sequence of the larger nucleic acid sequence they represent.

In various embodiments, a complete or partial genomic sequence can include at least, e.g., about 1 kb, 5 kb, 10 kb, 50 kb, 100 kb, 500 kb, 1 Mb, 2 Mb, 3 Mb, 4 Mb, 5 Mb, 10 Mb, 20 Mb, 50 Mb, 100 Mb, 500 Mb, 1,000 Mb, 2,000 Mb, 3,000 Mb, or more. In various embodiments, a complete genomic sequence can include a number of nucleotides equal to a canonical number of nucleotides for the genome of the relevant organism. In various embodiments, a complete genomic sequence can include a number of nucleotides within the range of the number of nucleotides typical for the genome of the relevant organism.

In various embodiments, a complete or partial plasmid sequence can include at least, e.g., about 1 kb, 5 kb, 10 kb, 50 kb, 100 kb, 200 kb, or more. In various embodiments, a complete plasmid sequence can include a number of nucleotides equal to a canonical number of nucleotides for the sequence of the relevant plasmid. In various embodiments, a complete genomic sequence can include a number of nucleotides within the range of the number of nucleotides typical for the relevant plasmid.

Genomic sequences, or plasmid sequences, of the present disclosure can include one or more sequences available in a publicly accessible database. Various publicly accessible databases include accessible genomic and plasmid sequence information (see, e.g., FIG. 19). One example of a publicly accessible database of genomic and/or plasmid sequence information is GenBank of the National Center for Biotechnology Information (NCBI). Another publicly accessible database of genomic and/or plasmid sequence information is the International Nucleotide Sequence Database Collaboration (INSDC) (available on the World Wide Web at ncbi.nlm.nih.gov/sra/) of the European Molecular Biology Laboratory (EMBL), the DNA Databank of Japan (DDBJ), and NCBI. Another example is the 1000 Genomes Project.

To provide just one example of the expansion of publicly accessible genomic sequence information resources, from August 2010 to August 2017, public databases expanded from about 19 Staphylococcus aureus genomic sequences to about 48,259 Staphylococcus aureus genomic sequences derived from about 4,155 independent studies. Most sequence data are deposited at the Sequence Read Archive at the US National Center for Biotechnology Information (NCBI), which is part of the INSDC. Of the S. aureus genomic sequences, about 84% (about 42,285) represented short DNA reads or small fragments. The remaining fraction (about 7,974; about 16%) were assembled into larger DNA segments and only about 2% (about 166/7,974) are gapless and fully-annotated. Therefore, fully assembled and annotated complete genomic sequences represent a minor fraction of S. aureus genomes available in NCBI.

Genomic sequences, or plasmid sequences, of the present disclosure can include sequences derived from biological samples and not found in a publicly accessible database. A biological sample can include, e.g., a laboratory sample or a clinical sample. A genomic sequence, or plasmid sequence, can be determined, e.g., by any of the various methods of DNA sequencing known in the art (e.g., high-throughput sequencing and/or multiplex sequencing).

A data structure can include (e.g., store) information related to genomic sequences and/or plasmid sequences of the present disclosure, including the sequences themselves. Thus, data structures of the present disclosure can include, without limitation, publicly accessible database of genomic sequence information, private structures including sequence information, structures including data directly input from high-throughput sequencing systems, and combinations thereof.

Genomic sequences representative of double-stranded DNA can be provided in the form of either strand (sometimes referred to as “Watson” and “Crick” strands or as “5′” and “3′” strands). The two strands are generally understood to be complementary, such that the sequence of either strand discloses the sequence of the other.

A plurality of complete or partial genomic sequences and/or plasmid sequences can be acquired, included in a data structure, and obtained from the data structure according to various techniques known in the art. Genomic sequences and/or plasmid sequences obtained or obtainable from a data structure can be sequences from existing records (e.g., in public databases) and/or sequences acquired by sequencing of samples. In various embodiments, a data structure can include differing sequences that represent or are associated with a particular source (e.g., a particular species, e.g., humans or a particular pathogen species). In various embodiments, each differing sequence representative of or associated with a particular source can be referred to as a strain. In various embodiments, it is advantageous to obtain from a data structure a plurality of sequences representative of or associated with a particular source so that obtained sequences can be compared and/or contrasted, e.g., according to various methods and systems disclosed herein.

Extraction of Coding Sequences and Encoded Amino Acid Sequences

Genomic and plasmid sequences of the present disclosure can include coding sequences. Various genomes and plasmids include nucleotide sequences that encode amino acids of proteins expressible from the genome or plasmid (which nucleotide sequences can be referred to as coding sequences) and nucleotide sequences that do not encode amino acids of proteins expressible from the sequence (which nucleotide sequences can be referred to as non-coding sequences). Coding sequences can be read in triplets referred to as codons, each of which codons encodes an amino acid. Thus, coding sequences of the present disclosure are sequences that consist of codons and encode a protein or a portion thereof. Non-coding sequences (e.g., promoters or introns) are in some cases adjacent to and/or interspersed with coding sequences. Coding sequences can be distinguished from non-coding sequences by a variety of techniques known in the art, including without limitation by the number of contiguous and/or in-frame codons encoding amino acids and/or by comparison to known sequences such as known coding sequences or known proteins encoded by coding sequences. Various methods of extracting (identifying and/or isolating) coding sequences are known in the art. Various methods of extracting coding sequences include analyzing a provided sequence for open reading frames that can include, among other features, a contiguous series of codons that does not include a termination codon, e.g., a contiguous series of at least about 20, 30, 40, 50, 60, 70, 80, 90, 100, 125, 150, 175, 200, 250, or 300 or more codons that does not include a termination codon. In some embodiments, a sequence in a publicly accessible database is associated with annotation information that demarcates the locations of coding sequences. Thus, either or both of database annotation and any of the various methods known in the art can be used to extract coding sequences from genomic and plasmid sequences.

Once a coding sequence has been extracted, the sequence of amino acids encoded by the coding sequence can be determined by applying the genetic code. Each codon that is not a stop codon corresponds to a particular amino acid. The genetic code can differ between organisms. Accordingly, a genetic code appropriate to the source and/or context of a genomic sequence or plasmid coding sequence can be applied when converting the coding sequence to an amino acid sequence. A nucleic sequence has been converted to an amino acid sequence by applying a genetic code can be referred to as a translation of the nucleic acid sequence.

The human genetic code, as with other genetic codes, can be represented as a DNA codon table, as seen in Table 1. Most codons encode particular amino acids, while several codons encode a “STOP” signal that does not code for any amino acid. Table 1 includes certain general conventions applied in the representation of nucleic acid and amino acid sequences. With reference to nucleic acid sequences, the letters A, C, G, and T respectively indicate adenine (A), cytosine (C), guanine (G), and thymine (T). With reference to amino acid sequences, each of twenty amino acids can be represented by a particular letter or set of three letters as follows: Alanine (A; Ala), Arginine (R; Arg), Asparagine (N; Asn), Aspartic Acid (D; Asp), Cysteine (C; Cys), Glutamic Acid (E; Glu), Glutamine (Q; Gln), Glycine (G; Gly), Histidine (H; His), Isoleucine (I; Ile), Leucine (L; Leu), Lysine (K; Lys), Methionine (M; Met), Phenylalanine (F; Phe), Proline (P; Pro), Serine (S; Ser), Threonine (T; Thr), Tryptophan (W; Trp), Tyrosine (Y; Tyr), Valine (V; Val).

TABLE 1

| | T | | | C | | | A | | | G | | |
|---|---|---|---|---|---|---|---|---|---|---|---|---|
| T | TTT | Phe | F | TCT | Ser | S | TAT | Tyr | Y | TGT | Cys | C |
| | TTC | | | TCC | | | TAC | | | TGC | | |
| | TTA | Leu | L | TCA | | | TAA | STOP | | TGA | STOP | |
| | TTG | | | TCG | | | TAG | | | TGG | Trp | W |
| C | CTT | Leu | L | CCT | Pro | P | CAT | His | H | CGT | Arg | R |
| | CTC | | | CCC | | | CAC | | | CGC | | |
| | CTA | | | CCA | | | CAA | Gln | Q | CGA | | |
| | CTG | | | CCG | | | CAG | | | CGG | | |
| A | ATT | Ile | I | ACT | Thr | T | AAT | Asn | N | AGT | Ser | S |
| | ATC | | | ACC | | | AAC | | | AGC | | |
| | ATA | | | ACA | | | AAA | Lys | K | AGA | Arg | R |
| | ATG | Met | M | ACG | | | AAG | | | AGG | | |
| G | GTT | Val | V | GCT | Ala | A | GAT | Asp | D | GGT | Gly | G |
| | GTC | | | GCC | | | GAC | | | GGC | | |
| | GTA | | | GCA | | | GAA | Glu | E | GGA | | |
| | GTG | | | GCG | | | GAG | | | GGG | | |

Data Generated from Pairwise Comparison of Sequences

In certain embodiments, methods and systems of the present disclosure include determining measurements to characterize alignment between sequences. Example measurements include percent identity, percent coverage, coverage length, percent identity over a predetermined coverage length, E-value, number of mutations, percent mutation, and phylogeny (e.g., phylogenetic groupings and/or phylogenetic relationships), all of which are discussed in more detail herein. It has been found that characterizing alignment using both a measure of coverage (e.g., percent coverage and/or coverage length) and a measure of identity (e.g., percent identity, percent identity over a predetermined coverage length, number of mutations, and percent mutation) efficiently and effectively achieves a high number of pairwise comparisons that can be used, for example, in identifying properly matched sequences in an assessment of conservation. Pairwise comparison can be used to evaluate the overall relatedness between polymeric sequences, e.g., between nucleic acid sequences (e.g., DNA molecules and/or RNA molecules) and/or between amino acid sequences. In various methods and systems provided herein, pairwise comparison is used to evaluate the overall relatedness between extracted coding sequences and/or translations thereof. In some embodiments, a pairwise comparison of two sequences is between a query sequence and a subject sequence (e.g., a reference sequence), the comparison including alignment and determination of one or more, or all, of percent identity, percent coverage, coverage length, percent identity over a predetermined coverage length, E-value, number of mutations, percent mutation, and/or phylogeny (e.g., phylogenetic groupings and/or phylogenetic relationships). In various embodiments, a subject sequence such as a reference sequence can be a baseline to which a query sequence is compared. Generally, query sequences and subject sequences refer respectively to collections of one or more sequences, where query sequences are pairwise compared with subject sequences. In some embodiments, query sequences are not compared to query sequences and subject sequences are not compared to subject sequences, except insofar as query sequences and subject sequences have the same sequence (e.g., in embodiments in which the query sequences and the subject sequences are identical collections of sequences). A subject sequence can be or include a reference sequence. A reference sequence can be a complete or partial genomic sequence that is representative of corresponding complete or partial genomic sequences of a population, species, strain, organism, or the like, e.g., that include one or more particular genes or portions thereof and/or that encode one or more proteins or portions thereof. A reference sequence can be selected and/or used as a representative sequence based on, without limitation, any of one or more of sequence availability, public accessibility, historical context, convention, canon, standard practices, statistical analysis, practical considerations, or user preference. As disclosed herein, data generated from pairwise comparison of sequences can include one or more, or all, of percent identity, percent coverage, coverage length, percent identity over a predetermined coverage length, E-value, number of mutations, percent mutation, and/or phylogeny (e.g., phylogenetic groupings and/or phylogenetic relationships), each of which provides distinct information relating to analyzed sequences.

In performing pairwise comparisons of query sequences with reference sequences, it is found herein to be remarkably efficient and effective to determine both a measurement of identity and a measurement of coverage for a given pairwise comparison, then use both measurements in categorizing the query sequences (e.g., coding sequences) into two or more groups, e.g., for identifying properly comparable sequence portions in an assessment of conservation of one or more amino acid sequences or portions thereof. Examples of measurements of identity include percent identity; percent identity/predetermined coverage length; number of mutations; and percent mutation (e.g., single nucleotide polymorphisms SNP/size). Examples of measurements of coverage include percent coverage and coverage length.

Methods for aligning two provided sequences include algorithms and/or commercially available computer programs such as BLASTN for nucleotide sequences and BLASTP, gapped BLAST, and PSI-BLAST for amino acid sequences. Calculation of a measure of coverage and a measure of identity may follow the alignment of the two sequences (or the complement of one or both sequences) using one or more of these alignment algorithms. In certain embodiments, gaps are introduced in one or both of a first and a second sequence for optimal alignment, and non-identical sequences can be disregarded for comparison purposes. Alignment refers to the process, or result, of matching up nucleotide or amino acid residues of two or more sequences to achieve a maximal level of percent identity and, in some embodiments (e.g., in the alignment of amino acid sequences), to maximize conservation of physico-chemical properties.

After alignment, nucleotides or amino acids at corresponding positions of a first and a second sequence can be compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Accordingly, determination of percent identity requires determining the identity or non-identity of aligned positions. The determination of percent identity between two sequences can be accomplished using a computational algorithm, such as BLAST (basic local alignment search tool).

A percent identity can express the fraction of positions within an aligned sequence that have the same residue in both of the aligned sequences. In some embodiments, two sequences are considered to be substantially identical if at least about 50%, 55%, 60%, 65%, 70%, 75%, 80%, 85%, 90%, 91%, 92%, 93%, 94%, 95%, 96%, 97%, 98%, 99% or more of their corresponding residues are identical over a relevant sequence. Sequences can be substantially similar if they differ by a conservative substitution, e.g., by nucleotide substitution that does not change an encoded amino acid sequence, or by amino acid substitution in which the substituted amino acid has similar structural or functional characteristics (e.g., replacement of a hydrophobic, hydrophilic, polar, or non-polar type amino acid with a different amino acid of the same type).

Each sequence analyzed in a pairwise comparison can also be evaluated according to the percent of a first sequence that is covered by the alignment with the second sequence (i.e., the percent of the first sequence that is aligned with the second sequence, which can be referred to as coverage or percent coverage) (e.g., % of subject sequence length aligned with query sequence or % of query sequence length aligned with subject sequence).

Alignment of two sequences can generate a coverage length and/or a percent coverage. In the alignment of a first sequence and a second sequence, coverage length refers to the number of units (e.g., nucleotides or amino acids) that are aligned. For avoidance of doubt, in calculating coverage length, a pair of corresponding positions (i.e., a nucleotide or amino acid of a first sequence and the correspondingly positioned nucleotide or amino acid of a second sequence) count as one unit of coverage length. In the alignment of a first sequence and a second sequence, percent coverage refers to the percent of the query that is included in the alignment of the sequences. Percent coverage can refer to the percent of nucleotide or amino acids in a subject sequence that are aligned with corresponding nucleotides or amino acids of a query sequence, regardless of whether aligned nucleotides or amino acids are identical or non-identical. Percent coverage can also refer to the percent of nucleotide or amino acids in a query sequence that are aligned with corresponding nucleotides or amino acids of a subject sequence, regardless of whether aligned nucleotides or amino acids are identical or non-identical. In various methods and systems provided herein, percent coverage refers in particular to the percent of nucleotide or amino acids in a subject sequence that are aligned with corresponding nucleotides or amino acids of a query sequence, regardless of whether aligned nucleotides or amino acids are identical or non-identical. Percent coverage can be determined for both contiguous and gapped alignments.

In various embodiments, at least because percent identity is determined by comparison of aligned nucleotides or amino acids to determine the identity or non-identity of each aligned pair of nucleotides or amino acids, sequence gaps do not reduce percent identity. To provide one example for purposes of illustration, if a query sequence of 80 amino acids is aligned to a subject sequence of 100 amino acids, where the first 40 amino acids of the subject sequence align with perfect identity to the first 40 amino acids of the query sequence and the last 40 amino acids of the subject sequence align with perfect identity to the last 40 amino acids of the query sequence, the percent identity would be equal to 100% but the percent coverage would be 80%. Thus, in some embodiments, despite 100% identity, the query sequence would be categorized as partial or “lack of integrity,” falling in the threshold range of 70% to 95% coverage.

In various embodiments, alignment of two sequences can be used to determine a percent identity over a predetermined coverage length. A predetermined coverage length can be a number of nucleotides and/or amino acids, where percent identity over the predetermined coverage length can refer to percent identity between a query sequence and a subject sequence over any portion of an alignment thereof that has a length equal to the predetermined coverage length and/or greater than the predetermined coverage length. For the avoidance of doubt, the portion of the alignment can be any sufficiently long subset of nucleotides or amino acids of the alignment, such that a single alignment can include a plurality of sufficiently long portions for analysis, which portions can be overlapping, non-overlapping, adjacent, or non-adjacent. In various embodiments, a percent identity over a predetermined coverage length for an alignment of two sequences can be presented as the highest percent identity associated with any sufficiently long portion of the alignment.

Various techniques of calculating percent identity produce an Expect (E) value. For instance, determination of percent identity using BLAST produces an E-value. An E-value represents the likelihood that an alignment occurred by chance (e.g., rather than as a result of biologically meaningful similarity). E-value has been described by some sources as essentially a description of background noise. The closer an E-value is to zero, the more significant the alignment. E-value relates at least in part to the determined percent identity of the alignment and the length of the alignment. Broadly, shorter and lower percent identity alignments will have higher E-values than longer and higher percent identity alignments. An E-value can be used to rank a plurality of alignments or can be selected as a significance threshold for categorizing alignments, alone or in combination with other criteria.

In some embodiments, for each query sequence analyzed in a pairwise comparison, the number of sequence variations within an alignment can be determined relative to the subject sequence. A variation can be a difference between aligned positions of a first sequence and a second sequence, where the sequences are nucleic acid sequences or where the sequences are amino acid sequences (e.g., a difference between a query sequence and a subject sequence such as a reference sequence). A variation in a nucleic acid sequence or a variation in an amino acid sequence can be referred to herein as a mutation. A variation in a nucleic acid sequence can be a Single Nucleotide Polymorphism (“SNP”).

In some embodiments, for each query sequence analyzed in a pairwise comparison, the number of sequence variations between the query sequence and the subject sequence (i.e., the number of sequence positions within the alignment between query and subject that are non-matching) can be referred to as the “number of mutations.” In some embodiments, for each query sequence analyzed in a pairwise comparison, the number of sequence variations per nucleotide or amino acid of sequence coverage length can be determined. This ratio can be the number of sequence variations within an alignment over the length of the alignment (“percent mutation,” alternatively referred to herein as “mutation/size,” an example of which is “SNP/size”).

In some embodiments, results of pairwise comparison can be used to generate a phylogeny for one or more genomes, plasmids, genes, coding sequences, or translated coding sequences. In some embodiments, a phylogeny can be based on percent identity data generated by pairwise comparisons. In some embodiments, a phylogeny can be based on percent mutation data generated by pairwise comparisons. Tools and techniques for generating phylogenies from provided data are known in the art.

Genome-level or plasmid-level phylogenies can be generated using the percent identity or percent mutation pairwise comparison results for the most conserved subject sequences. For example, a genome-level or plasmid-level phylogeny can be based on about the top 1, top 2, top 3, top 4, top 5, top 10, top 20, top 25, top 50, top 100, top 1%, top 2%, top 5%, top 10%, top 15%, top 20%, top 25%, or top 50% of conserved pairwise-compared sequence (e.g., top genes, coding sequences, or translated coding sequence amino acid sequences). Conservation can be ranked based on the result of pairwise comparison using, e.g., percent identity or percent mutation data.

Any of one or more, or all, of percent identity, percent coverage, coverage length, percent identity over a predetermined coverage length, E-value, number of mutations, and/or percent mutation can represent the full length of a nucleic acid or amino acid alignment or one or more portions thereof. Exemplary portions of complete or partial genomic sequences can include, e.g., a gene, coding sequence, individual nucleotide, or set of contiguous nucleotides (e.g., about 1, 2, 3, 4, 5, 6, 7, 8, 9, 10, 20, 30, 40, 50, 100, 150, 200, 250, 500, 1,000, 1,500, 2,000, 2,500, 3,000, 5,000, 10,000, or more nucleotides). Exemplary portions of amino acid sequences can include, e.g., a protein, domain, individual amino acid, or set of contiguous amino acids (e.g., about 1, 2, 3, 4, 5, 6, 7, 8, 9, 10, 20, 30, 40, 50, 100, 150, 200, 250, 300, 350, 400, 450, or 500, or more amino acids). In some embodiments, a portion of a nucleic acid sequences can include a number of nucleotides that has a lower bound of about 1, 2, 3, 4, 5, 6, 7, 8, 9, 10, 20, 30, 40, 50, 100, 150, 200, 250, 500, 1,000, 1,500, 2,000, 2,500, or 3,000 nucleotides and an upper bound of about 50, 100, 150, 200, 250, 500, 1,000, 1,500, 2,000, 2,500, 3,000, 5,000, 10,000, or more nucleotides. In some embodiments, a portion of an amino acid sequence can include a number of amino acids that has a lower bound of about 1, 2, 3, 4, 5, 6, 7, 8, 9, 10, 20, 30, 40, 50, 100, 150, 200, 250, or 300 amino acids and an upper bound of about 10, 20, 30, 40, 50, 100, 150, 200, 250, 300, 350, 400, 450, or 500, or more amino acids. In various embodiments, each overlapping or adjacent non-overlapping portion of a nucleic acid or amino acid sequence can be individually analyzed. Accordingly, first and second aligned nucleotide sequences can have a total percent identity representing percent identity between all aligned nucleotides of the first and second aligned sequences, and can have one or more percent identities representing percent identity between a subset of the aligned nucleotides of the first and second aligned sequences. First and second aligned amino acid sequences can have a total percent identity representing percent identity between all aligned amino acids of the first and second aligned sequences, and can have one or more percent identities representing percent identity between a subset of the aligned amino acids of the first and second aligned sequences. The percent identity of a subset of the aligned nucleotides or amino acids can be a different percent than the total percent identity for all aligned nucleotides or amino acids.

In various embodiments, any of one or more, or all, of percent identity, percent coverage, coverage length, percent identity over a predetermined coverage length, E-value, number of mutations, and/or percent mutation can be displayed as a graph or heatmap. In various embodiments, at least one axis of a graph or heatmap includes sequences included in a pairwise comparison of sequences and at least one additional axis includes data generated by the pairwise comparison of sequences.

In some embodiments, a single collection of genomic sequences or a single collection of plasmid sequences is analyzed, where all members of the analyzed collection are compared in a pairwise manner (i.e., the single collection is used as both the query sequence collection and the reference sequence collection) to determine the percent identity, percent coverage, coverage length, percent identity over a predetermined coverage length, E-value, number of mutations, and/or percent mutation of each pairwise comparison. In some embodiments, a collection of genomic sequences or a collection of plasmid sequences is analyzed, where each member of the analyzed collection is compared to a subject sequence to determine the percent identity, percent coverage, coverage length, percent identity over a predetermined coverage length, E-value, number of mutations, and/or percent mutation of each comparison.

In some embodiments, each genomic or plasmid sequence of a collection can be of the same species. In some embodiments, each genomic or plasmid sequence of a collection can be or include a sequence representative of organism of the same genus, family, order, class, phylum, kingdom, or domain. In some embodiments, each genomic or plasmid sequence of a collection can be or include a sequence representative of the same gene or a portion thereof. In some embodiments, each genomic or plasmid sequence of the single collection can be or include a sequence representative of the same coding sequence or a portion thereof.

In certain embodiments, analysis includes two collections, each of which is a collection of genomic sequences or each of which is a collection of plasmid sequences. In such instances a first collection can be referred to as a subject, and the second collection can be referred to as a query. In certain embodiments including a subject collection and a query collection, each sequence of the query collection is compared in a pairwise manner to each sequence of the subject collection to determine the percent identity, percent coverage, coverage length, percent identity over a predetermined coverage length, E-value, number of mutations, and/or percent mutation of each comparison.

In some embodiments, analysis includes a single collection of sequences and each sequence is compared to the other in a pairwise manner such that, in at least certain embodiments, the single collection of sequences is both the subject and the query. Whether the sequences analyzed include a single collection of sequences or multiple collections such as a subject and a query, all sequences used in the analysis can be cumulatively together, or with respect to any subset thereof, referred to as input sequences.

In some embodiments, each genomic or plasmid sequence of a subject and/or of a query can be of the same species. In some embodiments, each genomic or plasmid sequence of the subject and/or of the query can be or include a sequence representative of organism of the same genus, family, order, class, phylum, kingdom, or domain. In some embodiments, each genomic or plasmid sequence of the subject and/or of the query can be or include a sequence representative of the same gene or a portion thereof. In some embodiments, each genomic or plasmid sequence of the subject and/or of the query can be or include a sequence representative of the same coding sequence or a portion thereof.

In some embodiments, one or more, or all, subject sequences can be comparable to one or more query sequences in that it is representative of the same species. In some embodiments, one or more, or all, subject sequences can be comparable to one or more query sequences in that it is from an organism of the same genus, family, order, class, phylum, kingdom, or domain. In some embodiments, one or more, or all, subject sequences can be comparable to one or more query sequences in that it is representative of the same gene or a portion thereof. In some embodiments, one or more, or all, subject sequences can be comparable to one or more query sequences in that it is representative of the same coding sequence or a portion thereof.

In some embodiments one or more, or all, subject sequences are available in, and/or from, a publicly accessible database. In some embodiments, one or more, or all, subject sequences are derived from biological samples and not found in a publicly accessible database. In some embodiments one or more, or all, query sequences are available in, and/or from, a publicly accessible database. In some embodiments, one or more, or all, query sequences are derived from biological samples and not found in a publicly accessible database. In some embodiments one or more, or all, subject sequences are available in, and/or from, a publicly accessible database; and one or more, or all, query sequences are derived from biological samples and not found in a publicly accessible database.

In some embodiments, initially input genomic or plasmid sequences are compared. In certain embodiments, extracted coding sequences of initially input genomic or plasmid sequences are compared. In certain embodiments, translations of extracted coding sequences of initially input genomic or plasmid sequences are compared. Accordingly, in certain embodiments, initially input query genomic or plasmid sequences are compared in a pairwise manner to initially input subject genomic or plasmid sequences. In certain embodiments, extracted coding sequences of initially input query genomic or plasmid sequences are compared in a pairwise manner to extracted coding sequences of initially input subject genomic or plasmid sequences. In certain embodiments, translations of extracted coding sequences of initially input query genomic or plasmid sequences are compared in a pairwise manner to translations of extracted coding sequences of initially input subject genomic or plasmid sequences.

Processing of Data Generated by Pairwise Comparisons: Combinations of Multiple Sequence Categorization Factors for Efficient Categorization of Sequences

The present disclosure includes use of data generated from pairwise sequence comparisons to efficiently categorize sequences. In various embodiments, data resulting from pairwise sequence comparisons includes percent identity, percent coverage, coverage length, percent identity over a predetermined coverage length, E-value, number of mutations, percent mutation, and/or phylogeny, any or all of which can be used individually or in combinations, e.g., in combinations set forth herein, as sequence categorization factors. Thus, in various embodiments, sequences can be categorized into categorized sequence groups, which categorized sequence groups can be based on one or more threshold values for one or more categorization factors, In various embodiments, categorization factors can be used to filter sequences out for purposes of any further analysis (or to otherwise exclude sequences from further consideration), e.g., where the filtering is based on threshold values of one or more categorization factors and/or filtering out of one or more categorized sequence groups, Conversely, in various embodiments, categorization factors can be used to select sequences for inclusion in further analyses, e.g., where the selection is based on threshold values of one or more categorization factors and/or selection of one or more categorized sequence groups, In various embodiments, data resulting from pairwise sequence comparisons, optionally together with the sequences of the analyzed sequences and/or available annotations, if any, can be compiled together, e.g., in a Got Table.

As disclosed herein, the pairwise sequence comparisons can be comparisons of nucleic acid coding sequences (e.g., extracted coding sequences) or comparisons of amino acid sequences (e.g., translations of extracted coding sequences). Accordingly, query sequences categorized according to methods and systems of the present disclosure can include nucleic acid coding sequences (e.g., extracted coding sequences) or amino acid sequences (e.g., translations of extracted coding sequences).

In various embodiments, sequences can be categorized, or filtered out for purposes of any further analysis, based on whether percent identity is equal to and/or below a threshold value. In various embodiments, sequences can be categorized, or selected for inclusion in further analysis, based on whether percent identity is equal to and/or above a threshold value. In various embodiments, an exemplary threshold percent identity can be equal to or at least about, e.g., 75%, 80%, 85%, 90%, 95%, 96%, 97%, 98%, 99%, or 100%. In various embodiments, a threshold percent identity can be within a range having a lower bound of, e.g., 75%, 80%, 85%, 90%, or 95% and an upper bound of, e.g., 80%, 85%, 90%, 95%, 96%, 97%, 98%, 99%, or 100%.

In various embodiments, sequences can be categorized, or filtered out for purposes of any further analysis, based on whether percent coverage is equal to and/or below a threshold value. In various embodiments, sequences can be categorized, or selected for inclusion in further analysis, based on whether percent coverage is equal to and/or above a threshold value. In various embodiments, an exemplary threshold percent coverage can be equal to or at least about, e.g., 75%, 80%, 85%, 90%, 95%, 96%, 97%, 98%, 99%, or 100%. In various embodiments, a threshold percent coverage can be within a range having a lower bound of, e.g., 75%, 80%, 85%, 90%, or 95% and an upper bound of, e.g., 80%, 85%, 90%, 95%, 96%, 97%, 98%, 99%, or 100%.

In various embodiments, sequences can be categorized, or filtered out for purposes of any further analysis, based on whether coverage length is equal to and/or below a threshold value. In various embodiments, sequences can be categorized, or selected for inclusion in further analysis, based on whether coverage length is equal to and/or above a threshold value. In various embodiments, an exemplary threshold coverage length can be equal to or at least about, e.g., 20, 25, 30, 35, 40, 45, 50, 75, 100, 125, 150, 175, or 200 nucleotides or amino acids. In various embodiments, a threshold coverage length can be within a range having a lower bound of, e.g., 20, 25, 30, 35, 40, 45, 50, 75, 100, 125, 150, or 175 nucleotides or amino acids and an upper bound of, e.g., 25, 30, 35, 40, 45, 50, 75, 100, 125, 150, 175, or 200 nucleotides or amino acids.

In various embodiments, sequences can be categorized, or filtered out for purposes of any further analysis, based on whether percent identity over a predetermined coverage length is equal to and/or below a threshold value. In various embodiments, sequences can be categorized, or selected for inclusion in further analysis, based on whether percent identity over a predetermined coverage length is equal to and/or above a threshold value. In various embodiments, an exemplary threshold percent identity over a predetermined coverage length can be, e.g., a percent identity that is equal to or at least about 75%, 80%, 85%, 90%, 95%, 96%, 97%, 98%, 99%, or 100% over a predetermined coverage length that is equal to or at least about 20, 25, 30, 35, 40, 45, 50, 75, 100, 125, 150, 175, or 200 nucleotides or amino acids. In various embodiments, a threshold percent identity over a predetermined coverage length can include a percent identity within a range having a lower bound of, e.g., 75%, 80%, 85%, 90%, or 95% and an upper bound of, e.g., 80%, 85%, 90%, 95%, 96%, 97%, 98%, 99%, or 100% and can include a coverage length within a range having a lower bound of, e.g., 20, 25, 30, 35, 40, 45, 50, 75, 100, 125, 150, or 175 nucleotides or amino acids and an upper bound of, e.g., 25, 30, 35, 40, 45, 50, 75, 100, 125, 150, 175, or 200 nucleotides or amino acids

In various embodiments, sequences can be categorized, or filtered out for purposes of any further analysis, based on based on whether E-value is equal to and/or above a threshold value. In various embodiments, sequences can be categorized, or selected for inclusion in further analysis, based on whether E-value is equal to and/or below a threshold value. In various embodiments, an exemplary threshold E-value can be equal to or at least about, e.g., 1e-50, 1e-40, 1e-30, 1e-20, 1e-10, 1e-9, 1e-8, 1e-7, 1e-6, 1e-5, 1e-4, 1e-3, or 1e-2. In various embodiments, a threshold E-value can be within a range having a lower bound of, e.g., 1e-50, 1e-40, 1e-30, 1e-20, 1e-10, 1e-9, 1e-8, 1e-7, 1e-6, 1e-5, 1e-4, or 1e-3 and an upper bound of, e.g., 1e-40, 1e-30, 1e-20, 1e-10, 1e-9, 1e-8, 1e-7, 1e-6, 1e-5, 1e-4, 1e-3, or 1e-2.

In various embodiments, sequences can be categorized, or filtered out for purposes of any further analysis, based on whether number of mutations is equal to and/or above a threshold value. In various embodiments, sequences can be categorized, or selected for inclusion in further analysis, based on whether number of mutations is equal to and/or below a threshold value. In various embodiments, an exemplary threshold number of mutations can be equal to or at least about, e.g., 1, 2, 3, 4, 5, 6, 7, 8, 9, 10, 11, 12, 13, 14, 15, 16, 17, 18, 19, 20, 25, 30, 35, 40, 45, or 50. In various embodiments, a threshold number of mutations can be within a range having a lower bound of, e.g., 1, 2, 3, 4, 5, 6, 7, 8, 9, 10, 11, 12, 13, 14, 15, 16, 17, 18, 19, 20, 25, 30, 35, 40, or 45 and an upper bound of, e.g., 2, 3, 4, 5, 6, 7, 8, 9, 10, 11, 12, 13, 14, 15, 16, 17, 18, 19, 20, 25, 30, 35, 40, 45, or 50.

In various embodiments, sequences can be categorized, or filtered out for purposes of any further analysis, based on whether percent mutation is equal to and/or above a threshold value. In various embodiments, sequences can be categorized, or selected for inclusion in further analysis, based on whether percent mutation is equal to and/or below a threshold value. In various embodiments, an exemplary threshold percent mutation can be equal to or at least about, e.g., 0%, 1%, 2%, 3%, 4%, 5%, 10%, 15%, 20%, or 25%. In various embodiments, a threshold percent mutation can be within a range having a lower bound of, e.g., 0%, 1%, 2%, 3%, 4%, 5%, 10%, 15%, or 20% and an upper bound of, e.g., 1%, 2%, 3%, 4%, 5%, 10%, 15%, 20%, or 25%.

In various embodiments, sequences can be categorized, or filtered out for purposes of any further analysis, based on phylogeny. In various embodiments, one or more clades are filtered out for purposes of any further analysis. In various embodiments, one or more clades are selected for inclusion in further analysis.

The present disclosure includes categorization of sequences based on two or more categorization factors from pairwise sequences comparisons. In various embodiments, categorization of sequences is based on two or more categorization factors selected from percent identity, percent coverage, coverage length, percent identity over a predetermined coverage length, E-value, number of mutations, and/or percent mutation. The present disclosure further includes embodiments in which categorized sequence groups are generated based on parameters (e.g., one or more threshold values) for two or more categorization factors. In some embodiments, each sequence category is assigned a numerical value. In various embodiments, a numerical value assigned to a sequence category can be a value that tracks with one or more categorization factors that measures the similarity between a query sequence and a subject sequence and/or can be referred to as a “similarity score.” Similarity scores can include any series of numerical values across any range, but in particular embodiments can include a range of 0 to 1, 0 to 10, or 0 to 100. Examples of similarity scores are provided herein.

In various embodiments, the present disclosure categorization of sequences based on two or more categorization factors including a first categorization factor that is a measurement of identity and a second categorization factor that is a measurement of coverage. In various embodiments, a measurement of identity can be selected from percent identity, percent identity over a predetermined coverage length, number of mutations, and percent mutation. In various embodiments, a measurement of coverage can be selected from percent coverage and coverage length.

In various embodiments, each sequence analyzed in a pairwise comparison can be assigned a similarity score based on a defined scoring system in which each sequence analyzed in a pairwise comparison is categorized or ranked according to percent coverage and number of sequence variations. For instance, sequences can be categorized and assigned similarity scores according to Table 2 below, in which each query sequence analyzed in a pairwise comparison with a particular subject sequence is assigned to the bin in which it falls that has the highest similarity score, based on data from comparison of the query sequence with the particular subject sequence:

TABLE 2

| | Number of | Assigned |
|---|---|---|
| Percent Coverage | Mutations | Similarity Score |
| ≥99% | =0 | 1 |
| ≥99% | <10 | 0.95 |
| ≥99% | ≥10 | 0.8 |
| ≥90% | (any) | 0.5 |
| ≥75% | (any) | 0.4 |
| >0% | (any) | 0.3 |
| =0% | (any) | 0 |

The values in Table 2 are further to be understood to provide ranges around provided values, e.g., as if each value in Table 2 were preceded by the term “about.” Similarity scores for sequences of some or all pairwise comparisons can be displayed in a matrix, heatmap, or graph such as a bar graph. For example, a matrix or heatmap that includes columns of cells and rows of cells could include a column for each subject sequence and a row for each query sequence, with each cell displaying a similarity score based on comparison of the query and the subject.

In some embodiments, pairwise sequence comparisons (and/or query sequences thereof) that fail to meet one or more threshold criteria or values (e.g., a threshold similarity score) can be filtered out for purposes of any further analysis (or otherwise excluded from further consideration). In some embodiments, data associated with pairwise sequence comparison of a particular query sequence and a particular subject sequence (and/or associated query sequences), where the data fail to meet one or more threshold criteria or values (e.g., a threshold similarity score), can be filtered out for purposes of any further analysis (or otherwise excluded from further consideration).

In some embodiments, pairwise sequence comparisons (and/or query sequences or subject sequences thereof) that fall into one or more particular categorized sequence groups as set forth herein can be filtered out for purposes of any further analysis (or otherwise excluded from further consideration). In some embodiments, data associated with pairwise sequence comparison of a particular query sequence and a particular subject sequence (and/or associated query sequences), where the data and/or sequences fall into one or more particular categorized sequence groups, can be filtered out for purposes of any further analysis (or otherwise excluded from further consideration).

Table 2 provides an exemplary categorization scheme that permits filtering of categorized sequence groups by similarity score. As set forth in the exemplary categorization scheme of Table 2, pairwise comparisons resulting in a percent coverage of at least about 99%, where the number of mutations is zero, are assigned a similarity score of 1; the remaining pairwise comparisons resulting in a percent coverage of at least about 99%, where the number of mutations is less than about 10, are assigned a similarity score of 0.95; the remaining pairwise comparisons resulting in a percent coverage of at least about 99%, where the number of mutations is at least 10, are assigned a similarity score of 0.8; the remaining pairwise comparisons resulting in a percent coverage that is at least about 90% but less than about 99%, including any number of mutations, are assigned a similarity score of 0.5; the remaining pairwise comparisons resulting in a percent coverage that is at least about 75% but less than about 90%, including any number of mutations, are assigned a similarity score of 0.4; the remaining pairwise comparisons resulting in a percent coverage that is at least about 0% but less than about 75%, including any number of mutations, are assigned a similarity score of 0.3; the remaining pairwise comparisons resulting in a percent coverage equal to 0%, including any number of mutations, are assigned a similarity score of 0.

In certain embodiments, any of one or more sequence comparisons categorized as set forth in Table 2 (or as categorized by another combined measure of coverage and identity) can be filtered out for purposes of any further analysis (or otherwise excluded from further consideration), e.g., by filtering to exclude sequence comparisons having an assigned similarity score less than 1, less than 0.95, less than 0.8, less than 0.5, less than 0.4, less than 0.3, or 0. In certain embodiments, one or more thresholds are applied to a pairwise comparison either before or after (or both before and after) being assigned to a category corresponding to a similarity score as set forth in Table 2 (or other similarity score that is a combination of a measure of coverage and a measure of identity). In certain embodiments, the one or more thresholds can include, for example, a minimum coverage length, a minimum percent coverage, a maximum E-value, a minimum percent identity, a minimum percent identity over a coverage length, a maximum number of mutations, and/or a maximum percent mutation. In certain embodiments, one or more thresholds are applied as an alternative to the filtering based on Table 2. In certain embodiments, the one or more thresholds can include, for example, a minimum coverage length, a minimum percent coverage, a maximum E-value, a minimum percent identity, a minimum percent identity over a coverage length, a maximum number of mutations, and/or a maximum percent mutation.

In some embodiments, in addition to or as an alternative to categorization and/or filtering based on Table 2, pairwise sequence comparisons demonstrating at least about 80% identity over coverage length of at least about 51 nucleotides or amino acids, with an E-value at or below about 0.001, can be included for further analysis, and/or pairwise sequence comparisons demonstrating less than about 80% identity and/or an alignment match length of about 50 or fewer nucleotides or amino acids and/or an E-value greater than about 0.001 are filtered out of the analysis.

Determination of Target Characteristics and/or Selection of Sequences with Target Characteristics

In various embodiments, methods and systems of the present disclosure can be used to determine whether one or more sequences display certain target characteristics, and/or to select sequences determined to have one or more target characteristics. As is further disclosed herein, exemplary target characteristics can include, without limitation, a target level of sequence conservation, level of sequence variability (e.g., across a collection of sequences and/or as compared to one or more subject sequences), or phylogenetic grouping,

In various embodiments, a categorization and/or filtering step is followed by one or more further steps for analysis of target characteristics, optionally including selection of sequences with target characteristics. In some embodiments in which nucleic acid sequences (e.g., extracted coding sequences) have been compared and categorized and/or filtered, analysis of target characteristics is carried out by translating the nucleic acids (e.g., extracted coding sequences) into amino acid sequences and optionally carrying out further pairwise comparisons of the amino acid sequences to one or more subject amino acid sequences. In some embodiments in which nucleic acid sequences (e.g., extracted coding sequences) have been compared and categorized and/or filtered, analysis of target characteristics is carried out by analysis of data from the pairwise nucleic acid sequence comparisons. In some embodiments in which amino acid sequences have been compared and categorized and/or filtered, analysis of target characteristics is carried out by analysis of data from the pairwise amino acid sequence comparisons.

Conservation and/or variability can be evaluated (e.g., measured or determined) with respect to any of one or more of genomes, plasmids, genes, coding sequences, or translated coding sequence amino acid sequences. Conservation and/or variability can be evaluated with respect to a subset of nucleotide positions of a coding sequence, e.g., a subset of nucleotide positions of the coding sequence that encode an amino acid domain. Conservation and/or variability can be evaluated with respect to one or more nucleotide positions within a coding sequence. Conservation and/or variability can be evaluated with respect to a subset of amino acid positions of a translated coding sequence amino acid sequence, e.g., a subset of amino acid positions that include an amino acid domain. Conservation and/or variability can be evaluated with respect to one or more amino acid positions within a translated coding sequence amino acid sequence.

A variety of approaches can be used for analysis of sequence conservation and/or variability. As disclosed herein, sequence conservation and/or variability can refer to a measure of the frequency of identity or non-identity of the nucleotide or amino acid at one or more corresponding positions across compared sequences. At least insofar as sequence conservation and sequence variability are both measures of the similarity between or among sequences, approaches for measuring one are generally applicable to measurement of both.

In some embodiments, sequence conservation and/or variability can be measured according to percent mutation. In some embodiments, sequence conservation and/or variability can be measured according to percent identity. In various embodiments, conservation and/or variability can be determined by a combination of a measure of identity and a measure of coverage. For example, in various embodiments, a sequence is identified as conserved if it meets both a threshold value of a measure of identity and a threshold value of a measure of coverage. In some embodiments, sequence conservation and/or variability can be measured according to percent mutation in combination with coverage length and/or percent coverage. In some embodiments, sequence conservation and/or variability can be measured according to percent identity in combination with coverage length and/or percent coverage. In some embodiments, sequence conservation and/or variability can be measured according to a similarity score (as exemplified, e.g., in Table 2).

In some embodiments, conservation of sequences corresponding to a particular subject coding sequence can be determined by averaging the percent identity of each sequence as compared to the particular subject coding sequence. In various embodiments, sequences with high conservation (low variability) are selected based on an average percent identity that is at least 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100%. In some embodiments, sequences with low conservation (high variability) are selected based on an average percent identity that is less than 99%, 98%, 97%, 96%, 95%, 94%, 93%, 92%, 91%, 90%, 85%, 80%, 75%, 70%, 65%, 60%, 55%, 50%, 40%, or 30%.

In various embodiments, sequences can be selected based on their measured level of conservation and/or variability. In some embodiments, sequences with high conservation (low variability) are selected, e.g., after ordering pairwise compared sequences according to a measure of conservation, selecting about the top 1, top 2, top 3, top 4, top 5, top 10, top 20, top 25, top 50, top 100, top 1%, top 2%, top 5%, top 10%, top 15%, top 20%, top 25%, or top 50% of conserved pairwise-compared sequence (e.g., top genes, coding sequences, or translated coding sequence amino acid sequences, or a subset or portion thereof). In some embodiments, sequences with low conservation (high variability) are selected, e.g., after ordering pairwise compared sequences according to a measure of conservation, selecting about the bottom 1, bottom 2, bottom 3, bottom 4, bottom 5, bottom 10, bottom 20, bottom 25, bottom 50, bottom 100, bottom 1%, bottom 2%, bottom 5%, bottom 10%, bottom 15%, bottom 20%, bottom 25%, or bottom 50% of conserved pairwise-compared sequence (e.g., bottom genes, coding sequences, translated coding sequence amino acid sequences, or a subset or portion thereof).

In various embodiments, sequence conservation is demonstrated by phylogenetic analysis. Various methods and programs for phylogenetic analysis include AncesTree, AliGROOVE, ape, Armadillo Workflow Platform, BAli-Phy, BATWING, BayesPhylogenies, BayesTraits, BEAST, BioNumerics, Bosque, BUCKy, Canopy, CITUP, ClustalW, Dendroscope, EzEditor, fastDNAml, FastTree 2, fitmodel, Geneious, HyPhy, IQPNNI, IQ-TREE, jModelTest 2, LisBeth, MEGA, Mesquite, MetaPIGA2, Modelgenerator, MOLPHY, MorphoBank, MrBayes, Network, Nona, PAML, ParaPhylo, PartitionFinder, PASTIS, PAUP*, phangorn, Phybase, phyclust, PHYLIP, phyloT, PhyloQuart, PhyloWGS, PhyML, phyx, POY, ProtTest 3, PyCogent, QuickTree, RAxML-HPC, RAxML-NG, SEMPHY, sowhat, SplitsTree, TNT, TOPALi, TreeGen, TreeAlign, Treefinder, TREE-PUZZLE, T-REX (Webserver), UGENE, Winclada, and Xrate,

Network Environment and Computing Devices

As shown in FIG. 37, an implementation of a network environment 3700 for use in providing systems, methods, and architectures as described herein is shown and described. In brief overview, referring now to FIG. 37, a block diagram of an exemplary cloud computing environment 3700 is shown and described. The cloud computing environment 3700 may include one or more resource providers 3702a, 3702b, 3702c (collectively, 3702). Each resource provider 3702 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 3702 may be connected to any other resource provider 3702 in the cloud computing environment 3700. In some implementations, the resource providers 3702 may be connected over a computer network 3708. Each resource provider 3702 may be connected to one or more computing device 3704a, 3704b, 3704c (collectively, 3704), over the computer network 3708.

The cloud computing environment 3700 may include a resource manager 3706. The resource manager 3706 may be connected to the resource providers 3702 and the computing devices 3704 over the computer network 3708. In some implementations, the resource manager 3706 may facilitate the provision of computing resources by one or more resource providers 3702 to one or more computing devices 3704. The resource manager 3706 may receive a request for a computing resource from a particular computing device 3704. The resource manager 3706 may identify one or more resource providers 3702 capable of providing the computing resource requested by the computing device 3704. The resource manager 3706 may select a resource provider 3702 to provide the computing resource. The resource manager 3706 may facilitate a connection between the resource provider 3702 and a particular computing device 3704. In some implementations, the resource manager 3706 may establish a connection between a particular resource provider 3702 and a particular computing device 3704. In some implementations, the resource manager 3706 may redirect a particular computing device 3704 to a particular resource provider 3702 with the requested computing resource.

FIG. 38 shows an example of a computing device 3800 and a mobile computing device 3850 that can be used to implement the techniques described in this disclosure. The computing device 3800 is intended to represent various forms of digital computers, such as laptops, desktops, workstations, personal digital assistants, servers, blade servers, mainframes, and other appropriate computers. The mobile computing device 3850 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 3800 includes a processor 3802, a memory 3804, a storage device 3806, a high-speed interface 3808 connecting to the memory 3804 and multiple high-speed expansion ports 3810, and a low-speed interface 3812 connecting to a low-speed expansion port 3814 and the storage device 3806. Each of the processor 3802, the memory 3804, the storage device 3806, the high-speed interface 3808, the high-speed expansion ports 3810, and the low-speed interface 3812, are interconnected using various busses, and may be mounted on a common motherboard or in other manners as appropriate. The processor 3802 can process instructions for execution within the computing device 3800, including instructions stored in the memory 3804 or on the storage device 3806 to display graphical information for a GUI on an external input/output device, such as a display 3816 coupled to the high-speed interface 3808. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, where a plurality of functions are described as being performed by a processor, this encompasses embodiments wherein the plurality of functions are performed by any number of processors (one or more) of any number of computing devices (one or more). Furthermore, where a function is described as being performed by a processor, this encompasses embodiments wherein the function is performed by any number of processors (one or more) of any number of computing devices (one or more) (e.g., in a distributed computing system).

The memory 3804 stores information within the computing device 3800. In some implementations, the memory 3804 is a volatile memory unit or units. In some implementations, the memory 3804 is a non-volatile memory unit or units. The memory 3804 may also be another form of computer-readable medium, such as a magnetic or optical disk.

The storage device 3806 is capable of providing mass storage for the computing device 3800. In some implementations, the storage device 3806 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 3802), perform one or more methods, such as those described above. The instructions can also be stored by one or more storage devices such as computer- or machine-readable mediums (for example, the memory 3804, the storage device 3806, or memory on the processor 3802).

The high-speed interface 3808 manages bandwidth-intensive operations for the computing device 3800, while the low-speed interface 3812 manages lower bandwidth-intensive operations. Such allocation of functions is an example only. In some implementations, the high-speed interface 3808 is coupled to the memory 3804, the display 3816 (e.g., through a graphics processor or accelerator), and to the high-speed expansion ports 3810, which may accept various expansion cards (not shown). In the implementation, the low-speed interface 3812 is coupled to the storage device 3806 and the low-speed expansion port 3814. The low-speed expansion port 3814, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 3800 may be implemented in a number of different forms, as shown in the figure. For example, it may be implemented as a standard server 3820, or multiple times in a group of such servers. In addition, it may be implemented in a personal computer such as a laptop computer 3822. It may also be implemented as part of a rack server system 3824. Alternatively, components from the computing device 3800 may be combined with other components in a mobile device (not shown), such as a mobile computing device 3850. Each of such devices may contain one or more of the computing device 3800 and the mobile computing device 3850, and an entire system may be made up of multiple computing devices communicating with each other.

The mobile computing device 3850 includes a processor 3852, a memory 3864, an input/output device such as a display 3854, a communication interface 3866, and a transceiver 3868, among other components. The mobile computing device 3850 may also be provided with a storage device, such as a micro-drive or other device, to provide additional storage. Each of the processor 3852, the memory 3864, the display 3854, the communication interface 3866, and the transceiver 3868, are interconnected using various buses, and several of the components may be mounted on a common motherboard or in other manners as appropriate.

The processor 3852 can execute instructions within the mobile computing device 3850, including instructions stored in the memory 3864. The processor 3852 may be implemented as a chipset of chips that include separate and multiple analog and digital processors. The processor 3852 may provide, for example, for coordination of the other components of the mobile computing device 3850, such as control of user interfaces, applications run by the mobile computing device 3850, and wireless communication by the mobile computing device 3850.

The processor 3852 may communicate with a user through a control interface 3858 and a display interface 3856 coupled to the display 3854. The display 3854 may be, for example, a TFT (Thin-Film-Transistor Liquid Crystal Display) display or an OLED (Organic Light Emitting Diode) display, or other appropriate display technology. The display interface 3856 may comprise appropriate circuitry for driving the display 3854 to present graphical and other information to a user. The control interface 3858 may receive commands from a user and convert them for submission to the processor 3852. In addition, an external interface 3862 may provide communication with the processor 3852, so as to enable near area communication of the mobile computing device 3850 with other devices. The external interface 3862 may provide, for example, for wired communication in some implementations, or for wireless communication in other implementations, and multiple interfaces may also be used.

The memory 3864 stores information within the mobile computing device 3850. The memory 3864 can be implemented as one or more of a computer-readable medium or media, a volatile memory unit or units, or a non-volatile memory unit or units. An expansion memory 3874 may also be provided and connected to the mobile computing device 3850 through an expansion interface 3872, which may include, for example, a SIMM (Single In Line Memory Module) card interface. The expansion memory 3874 may provide extra storage space for the mobile computing device 3850, or may also store applications or other information for the mobile computing device 3850. Specifically, the expansion memory 3874 may include instructions to carry out or supplement the processes described above, and may include secure information also. Thus, for example, the expansion memory 3874 may be provide as a security module for the mobile computing device 3850, and may be programmed with instructions that permit secure use of the mobile computing device 3850. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 3852), perform one or more methods, such as those described above. The instructions can also be stored by one or more storage devices, such as one or more computer- or machine-readable mediums (for example, the memory 3864, the expansion memory 3874, or memory on the processor 3852). In some implementations, the instructions can be received in a propagated signal, for example, over the transceiver 3868 or the external interface 3862.

The mobile computing device 3850 may communicate wirelessly through the communication interface 3866, which may include digital signal processing circuitry where necessary. The communication interface 3866 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 3868 using a radio-frequency. In addition, short-range communication may occur, such as using a Bluetooth®, Wi-Fi™, or other such transceiver (not shown). In addition, a GPS (Global Positioning System) receiver module 3870 may provide additional navigation- and location-related wireless data to the mobile computing device 3850, which may be used as appropriate by applications running on the mobile computing device 3850.

The mobile computing device 3850 may also communicate audibly using an audio codec 3860, which may receive spoken information from a user and convert it to usable digital information. The audio codec 3860 may likewise generate audible sound for a user, such as through a speaker, e.g., in a handset of the mobile computing device 3850. Such sound may include sound from voice telephone calls, may include recorded sound (e.g., voice messages, music files, etc.) and may also include sound generated by applications operating on the mobile computing device 3850.

The mobile computing device 3850 may be implemented in a number of different forms, as shown in the figure. For example, it may be implemented as a cellular telephone 3880. It may also be implemented as part of a smart-phone 3882, personal digital assistant, or other similar mobile device.

A further non-limiting schematic including certain components of an exemplary system is provided in FIG. 20.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. Machine-readable medium and computer-readable medium can refer to a computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. Machine-readable signal can refer to a signal used to provide machine instructions and/or data to a programmable processor.

In certain embodiments, the computer programs comprise one or more machine learning modules. Machine learning module can refer to a computer implemented process (e.g., function) that implements one or more specific machine learning algorithms. The machine learning module may include, for example, one or more artificial neural networks. In certain embodiments, two or more machine learning modules may be combined and implemented as a single module and/or a single software application. In certain embodiments, two or more machine learning modules may also be implemented separately, e.g., as separate software applications. A machine learning module may be software and/or hardware. For example, a machine learning module may be implemented entirely as software, or certain functions of a machine learning module may be carried out via specialized hardware (e.g., via an application specific integrated circuit (ASIC)).

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

Block Flow Diagrams of Various Embodiments

FIG. 39 is a block flow diagram 3900 of an exemplary method for identifying amino acid sequences as candidate antigens in the development of a therapy against a pathogen. Some or all of the steps may be performed in whole or in part by a processor of a computing device (e.g., executing software instructions).

In step 3910, a plurality of complete or partial genomic sequences of different strains of the pathogen are obtained (accessed). The sequences may come from public or private sequence databases, and/or from de novo sequencing reads. The plurality of sequences may include contigs that are merged to produce at least some of the complete or partial genomic sequences.

In step 3920, coding sequences are identified from the genomic sequences. In step 3930, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 3940, the coding sequences are converted into amino acid sequences, and in step 3950, the amino acid sequences are aligned. In certain embodiments, amino acid sequences are aligned by dint of the coding sequences having been aligned. In certain embodiments, the coding sequences are converted into amino acid sequences after the measures of similarity are computed, and in other embodiments, the coding sequences are converted into amino acid sequences before the measures of similarity are computed (e.g., where a measure of similarity is computed for each of a set of query amino acid sequences against a set of subject amino acid sequences).

In step 3960, aligned portions of the amino acid sequences are classified according to level of conservation of the sequence portion among the different strains of the pathogen represented by the plurality of genomic sequences accessed in step 3910. Of particular interest are those sequence portions that are highly conserved and, therefore, common to the various strains of the pathogen represented by the plurality of genomic sequences accessed in step 3910.

In step 3970, each amino acid sequence portion identified as highly conserved is checked to determine whether it is identical to a human protein sequence. Any highly conserved sequence identical to a human protein sequence is eliminated as a candidate antigen because of toxicity concerns. Other criteria may also be applied in identifying one or more final candidate antigens in the development of therapy against the pathogen, for example, the presence of a peptide signal, the protein annotation (or presence/absence thereof), the particular domain structure, and/or the presence of a transmembrane domain in the sequence, the latter of which may indicate whether the candidate antigen corresponds to a protein that is secreted or is exposed within a membrane and/or cell wall of the pathogen, thereby enhancing its potential value as a therapeutic against the pathogen. The method may additionally include the step of administering a polypeptide that encompasses the candidate antigen to an animal. Also, where the therapy is a vaccine, the method may include the step of non-clinically evaluating the candidate antigen for immunogenicity.

FIG. 40 is a block flow diagram 4000 of an exemplary method for identifying one or more conserved portions of coding sequences representative of a pathogen. Some or all of the steps may be performed in whole or in part by a processor of a computing device (e.g., executing software instructions).

In step 4010, a plurality of complete or partial genomic sequences of different strains of the pathogen are obtained (accessed) from a data structure. The sequences may come from public or private sequence databases, and/or from de novo sequencing reads. The plurality of sequences may include contigs that are merged to produce at least some of the complete or partial genomic sequences.

In step 4020, coding sequences are identified from the genomic sequences. In step 4030, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4040, the coding sequences are converted into amino acid sequences. In certain embodiments, the coding sequences are converted into amino acid sequences after they are categorized according to percent identity and percent coverage. In other embodiments, the coding sequences are converted into amino acid sequences before being categorized according to percent identity and percent coverage (e.g., where a measure of similarity is computed for each of a set of query amino acid sequences against a set of subject amino acid sequences).

In step 4050, portions of the amino acid sequences are classified according to level of conservation of the sequence portion among the different strains of the pathogen represented by the plurality of genomic sequences accessed in step 4010. Of particular interest are those sequence portions that are highly conserved and, therefore, common to the various strains of the pathogen represented by the plurality of genomic sequences accessed in step 4010.

FIG. 41 is a block flow diagram 4100 of an exemplary method for identifying whether an isolated pathogen is representative of a circulating strain. Some or all of the steps may be performed in whole or in part by a processor of a computing device (e.g., executing software instructions).

In step 4110, a plurality of complete or partial genomic sequences of a circulating strain of the pathogen are obtained (accessed). The sequences may come from public or private sequence databases, and/or from de novo sequencing reads. The plurality of sequences may include contigs that are merged to produce at least some of the complete or partial genomic sequences.

In step 4120, one or more conserved (e.g., highly conserved) portions of sequences of the circulating strain are identified. In certain embodiments, sequences of the circulating strain are categorized according to percent identity and percent coverage. For example, for each of a set of query coding sequences being compared against a set of subject sequences (where both “query” and “subject” sequences are of the circulating strain of the pathogen), measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4130, a plurality of complete or partial genomic sequences of the isolated pathogen are obtained (accessed). For example, the sequences of the isolated pathogen may come from de novo sequencing reads (e.g., high throughput sequencing reads of a biological sample obtained from a patient suffering from an infection). In certain embodiments these sequences may be analyzed as above to identify which portions are conserved and properly representative of the isolated pathogen.

In step 4140, one or more sequences of the isolated pathogen (or portions thereof) is/are compared against the one or more conserved (e.g., highly conserved) portions of sequences of the circulating strain identified in step 4120, thereby identifying whether the isolate pathogen is representative of (e.g., common to, an incidence of) the circulating strain.

FIG. 42 is a block flow diagram of an exemplary method for identifying an amino acid sequence as a candidate antibiotic resistance marker (e.g., in the development of a therapy against a pathogenic bacterium), according to an illustrative embodiment. Some or all of the steps may be performed in whole or in part by a processor of a computing device (e.g., executing software instructions).

In step 4210, a plurality of complete or partial genomic sequences of a pathogenic bacterium are obtained (accessed) from a data structure. The sequences may come from public or private sequence databases, and/or from de novo sequencing reads. The plurality of sequences may include contigs that are merged to produce at least some of the complete or partial genomic sequences.

In step 4220, coding sequences are identified from the plasmid sequences. In step 4230, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4240, the coding sequences are converted into amino acid sequences, and in step 4250, the amino acid sequences are aligned. In certain embodiments, amino acid sequences are aligned by dint of the coding sequences having been aligned. In certain embodiments, the coding sequences are converted into amino acid sequences after the measures of similarity are computed, and in other embodiments, the coding sequences are converted into amino acid sequences before the measures of similarity are computed (e.g., where a measure of similarity is computed for each of a set of query amino acid sequences against a set of subject amino acid sequences).

In step 4260, aligned portions of the amino acid sequences are classified according to level of conservation of the sequence portion among the plurality of plasmid sequences accessed in step 4210. Of particular interest are those sequence portions that are highly conserved and, therefore, common to the plasmids of the pathogen represented by the plurality of genomic sequences accessed in step 4210.

In step 4270, one or more sequence portions identified as conserved (e.g., highly conserved) are selected as a candidate antibiotic resistance marker. Other criteria may also be applied in identifying the candidate antibiotic resistance marker, for example, the presence of a peptide signal, the protein annotation (or presence/absence thereof), the particular domain structure, and/or the presence of a transmembrane domain in the sequence. The method may additionally include the step of administering a polypeptide that encompasses the candidate antibiotic resistance marker to an animal. Also, where the therapy is a vaccine, the method may include the step of non-clinically evaluating the polypeptide for immunogenicity.

FIG. 43 is a block flow diagram 4300 of an exemplary method for identifying one or more conserved portions of coding sequences representative of a plasmid, according to an illustrative embodiment. Some or all of the steps may be performed in whole or in part by a processor of a computing device (e.g., executing software instructions).

In step 4310, a plurality of complete or partial plasmid sequences of a pathogenic bacterium are obtained (accessed) from a data structure. The sequences may come from public or private sequence databases, and/or from de novo sequencing reads. The plurality of sequences may include contigs that are merged to produce at least some of the complete or partial genomic sequences.

In step 4320, coding sequences are identified from the plasmid sequences. In step 4330, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4340, the coding sequences are converted into amino acid sequences. In certain embodiments, the coding sequences are converted into amino acid sequences after they are categorized according to percent identity and percent coverage. In other embodiments, the coding sequences are converted into amino acid sequences before being categorized according to percent identity and percent coverage (e.g., where a measure of similarity is computed for each of a set of query amino acid sequences against a set of subject amino acid sequences).

In step 4350, portions of the amino acid sequences are classified according to level of conservation of the sequence portion among the plurality of plasmid sequences accessed in step 4310. Of particular interest are those sequence portions that are highly conserved and, therefore, common to the plasmids of the pathogen represented by the plurality of genomic sequences accessed in step 4310.

FIG. 44 is a block flow diagram of an exemplary method for identifying a mass-to-charge ratio of a peptide representative of a pathogen, for example, to identify mass spectrometry targets for such pathogen-representative peptides. Some or all of the steps may be performed in whole or in part by a processor of a computing device (e.g., executing software instructions).

In step 4410, a plurality of complete or partial genomic sequences of different strains of the pathogen are obtained (accessed). The sequences may come from public or private sequence databases, and/or from de novo sequencing reads. The plurality of sequences may include contigs that are merged to produce at least some of the complete or partial genomic sequences.

In step 4420, coding sequences are identified from the genomic sequences, and in step 4430, coding sequences are converted to amino acid sequences. In step 4440, one or more conserved portions of the amino acid sequences are identified. For example, sequences may be categorized according to percent identity and percent coverage. For example, for each of a set of query sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. In certain embodiments, coding sequences are converted into amino acid sequences after the measures of similarity are computed, and in other embodiments, the coding sequences are converted into amino acid sequences before the measures of similarity are computed (e.g., where a measure of similarity is computed for each of a set of query amino acid sequences against a set of subject amino acid sequences). A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4450, the mass-to-charge ratio of one or more of the sequence portions identified as conserved is determined. This is useful, for example, to identify mass spectrometry targets for the corresponding pathogen-representative peptides, such that they can be identified by mass spectrometry.

FIG. 45 is a block flow diagram of an exemplary method for identifying amino acid sequences as candidate antigens in the development of a therapy against a pathogen. Some or all of the steps may be performed in whole or in part by a processor of a computing device (e.g., executing software instructions).

In step 4510, a plurality of complete or partial genomic sequences of different strains of the pathogen are obtained (accessed). The sequences may come from public or private sequence databases, and/or from de novo sequencing reads. The plurality of sequences may include contigs that are merged to produce at least some of the complete or partial genomic sequences.

In step 4520, coding sequences are identified from the genomic sequences. In step 4530, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4540, the coding sequences are converted into amino acid sequences. In certain embodiments, the coding sequences are converted into amino acid sequences after the measures of similarity are computed, and in other embodiments, the coding sequences are converted into amino acid sequences before the measures of similarity are computed (e.g., where a measure of similarity is computed for each of a set of query amino acid sequences against a set of subject amino acid sequences).

In step 4550, portions of the amino acid sequences are classified according to level of conservation of the sequence portion among the different strains of the pathogen represented by the plurality of genomic sequences accessed in step 4510. Of particular interest are those sequence portions that are highly conserved and, therefore, common to the various strains of the pathogen represented by the plurality of genomic sequences accessed in step 4510.

In step 4560, each amino acid sequence portion identified as highly conserved is checked to determine whether it is identical to a human protein sequence. Any highly conserved sequence identical to a human protein sequence is eliminated as a candidate antigen because of toxicity concerns. Other criteria may also be applied in identifying one or more final candidate antigens in the development of therapy against the pathogen, for example, the presence of a peptide signal, the protein annotation (or presence/absence thereof), the particular domain structure, and/or the presence of a transmembrane domain in the sequence, the latter of which may indicate whether the candidate antigen corresponds to a protein that is secreted or is exposed within a membrane and/or cell wall of the pathogen, thereby enhancing its potential value as a therapeutic against the pathogen. The method may additionally include the step of administering a polypeptide that encompasses the candidate antigen to an animal. Also, where the therapy is a vaccine, the method may include the step of non-clinically evaluating the candidate antigen for immunogenicity.

FIG. 46 is a block flow diagram of an exemplary method 4600 for identifying an amino acid sequence as a candidate antibiotic resistance marker, according to an illustrative embodiment. Some or all of the steps may be performed in whole or in part by a processor of a computing device (e.g., executing software instructions).

In step 4610, a plurality of complete or partial genomic sequences of a pathogenic bacterium are obtained (accessed) from a data structure. The sequences may come from public or private sequence databases, and/or from de novo sequencing reads. The plurality of sequences may include contigs that are merged to produce at least some of the complete or partial genomic sequences.

In step 4620, coding sequences are identified from the plasmid sequences. In step 4630, the coding sequences are categorized according to percent identity and percent coverage. For example, for each of a set of query coding sequences being compared against a set of subject sequences, measures of similarity between the query coding sequence and each subject sequence are computed, where each of the measures of similarity is a function of (i) a percent identity between the query sequence and subject sequence and (ii) a percent coverage between the query sequence and subject sequence, In certain embodiments, a threshold involving both (i) and (ii) is applied. In some cases, an absolute (as opposed to relative) number of mutations is considered equivalent to a “percent identity”. The set of query sequences can be the same as the set of subject sequences, or they can be different sets or partially overlapping sets. A matrix of the measures of similarity may be graphically rendered. For example, a heat map of the similarity measurements may be graphically displayed, e.g., where x and y axes represent sequences and the intensity or color in a given x-y position represents the similarity measurement between the corresponding two sequences.

In step 4640, the coding sequences are converted into amino acid sequences. In certain embodiments, the coding sequences are converted into amino acid sequences after the measures of similarity are computed, and in other embodiments, the coding sequences are converted into amino acid sequences before the measures of similarity are computed (e.g., where a measure of similarity is computed for each of a set of query amino acid sequences against a set of subject amino acid sequences).

In step 4650, portions of the amino acid sequences are classified according to level of conservation of the sequence portion among the plurality of plasmid sequences accessed in step 4610. Of particular interest are those sequence portions that are highly conserved and, therefore, common to the plasmids of the pathogen represented by the plurality of genomic sequences accessed in step 4610.

In step 4660, one or more sequence portions identified as conserved (e.g., highly conserved) are selected as a candidate antibiotic resistance marker. Other criteria may also be applied in identifying the candidate antibiotic resistance marker, for example, the presence of a peptide signal, the protein annotation (or presence/absence thereof), the particular domain structure, and/or the presence of a transmembrane domain in the sequence. The method may additionally include the step of administering a polypeptide that encompasses the candidate antibiotic resistance marker to an animal. Also, where the therapy is a vaccine, the method may include the step of non-clinically evaluating the polypeptide for immunogenicity.

Elements of different implementations described herein may be combined to form other implementations not specifically set forth above. Elements may be left out of the methods, processes, computer programs, databases, etc. described herein without adversely affecting their operation. Various separate elements may be combined into one or more individual elements to perform the functions described herein.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader—the presence and/or placement of a header is not intended to limit the scope of the subject matter described herein.

Applications

Methods and Systems of the present disclosure that characterize sequence conservation between, among, and/or of subsets of residues within, input sequences are useful in a variety of analytic and therapeutic applications. Various uses of methods and systems of characterizing sequence conservation are provided herein. For instance, methods and systems disclosed herein can be used to identify the therapeutic relevance of uncharacterized sequences, e.g., based on sequence conservation characteristics. Non-limiting examples of the utility of methods and systems disclosed herein are provided.

Identification of Antigens for Selection of Anti-Antigen Antibodies

Among examples of a particular species, such as a pathogen species, genomic and plasmid nucleic acid sequences, including coding sequences, can vary. In many instances, variability in nucleic acid sequences derived from members of a particular species can be revealed by analysis of publicly available genomic sequences and/or other genomic sequences, such non-public sequencing data. Successful analysis of the growing volume of disparate sequence information is increasingly challenging, as the number of sequences deposited in publicly accessible databases alone is continually growing. Methods and systems of the present disclosure address this difficulty by providing a systematic methods of analyzing conservation characteristics of input sequences.

Conserved sequences of pathogen genomes may be preferable to non-conserved sequences of pathogen genomes as a source of antigens for use in production of anti-pathogen therapeutics. Identification and/or characterization of an antigen can be or include identification and/or characterization of an epitope. Antigens can be or include epitopes, and that one or more characteristics disclosed herein as useful in the identification of antigen are equally useful for identification of epitopes. At least one reason is that a therapeutic antibody or other drug molecule that binds or otherwise interacts with a sequence that is relatively conserved within a relevant pathogen population will necessarily be more likely to have a therapeutic benefit across a broader range of members of the pathogen species, and thus in patients suffering therefrom. Accordingly, sequences identified by methods and systems of the present disclosure that are conserved in a relevant pathogen population are identified as candidate antigens for development of therapeutic antibodies or as targets for other therapeutic modalities, such as small molecule drugs. Certain methods for the development of antibodies against therapeutic antigens are known in the art, and can include, to provide just one example, immunization of an antibody-generating organism with an antigen of interest.

In various embodiments, sequences identified as conserved can be further narrowed down to identify therapeutically relevant targets by secondary considerations. One secondary consideration is whether an identified candidate therapeutic target is identical to a known human sequences. Whether an identified sequence is identical to a known human sequence can be determined using publicly available databases and search tools. Various embodiments of the presently disclosed methods and systems include removal from among candidate therapeutic targets (e.g., from a list of candidate antigens) of candidate therapeutic targets that are identical to known human sequences. At least one reason for removal of sequences identical to known human sequences is that development of a drug (e.g., an antibody) that targets such a sequence could display clinically detrimental or otherwise undesired interactions with non-target human cells and/or proteins.

Additional examples of secondary considerations include protein annotations, functions, and/or the presence or absence of protein domains. Examples of protein domains include signal sequences, domains known to cause or be associated with secretion, domains characteristic of cell membrane proteins, characteristics indicative of extracellular exposure of a sequence at a cell membrane or cell wall, or other structural features. Extracellular exposure of a sequence facilitates interaction of therapeutic agents with the sequence, and is therefore a characteristic that may be desirable in a therapeutic target.

In certain embodiments, the above information, e.g., the identification of candidate antigens via the methods presented herein, is used in the development of one or more compositions (or identification of one or more new and/or existing compositions) for the treatment of a pathogen-caused disease. In certain embodiments, a therapy involving multiple drug compositions (e.g., a drug cocktail) is identified and/or developed. For example, the methods presented herein can be used to select for the best one or more pathogen-neutralizing antibodies that can be used in a drug (e.g., a drug cocktail) for the treatment of a pathogen-caused disease, such as COVID-19. In some embodiments, the drug is not a treatment for a disease but rather a stop-gap, e.g., for use in a pandemic, to enhance the ability of a human body (e.g., an immuno-compromised or otherwise vulnerable individual) to fight off infection, e.g., until a vaccine is developed. In some embodiments, the drug interferes with the functioning of the pathogen (e.g., a virus such as SARS-CoV2) to prevent or reduce damage caused by the virus to the human body, e.g., thereby reducing the need for a patient to use a ventilator and/or other respiratory devices. In some embodiments, the drug is a treatment customized for a particular individual or group of individuals. In certain embodiments, mice or other animals may be used for the manufacture of a composition for treatment of a pathogen-caused disease, where information produced via the computer-implemented methods presented herein is used in such manufacture. For example, mice or other animals may be injected with a virus (or portion thereof) for generating human antibodies that can be manufactured and administered to one or more patients. In certain embodiments, it is possible to proceed from identification of a sequence of a virus or other pathogen to production of an antibody that can be manufactured at scale using the methods presented herein.

In certain embodiments, the methods presented herein are used to evaluate coding sequences of a nucleic acid that encodes a protein, conserved sequences of a nucleic acid sequence that encodes a protein, non-conserved sequences (sequences characterized by variation) of a nucleic acid that encodes a protein, conserved domains within a particular protein, and/or non-conserved domains (sections characterized by variation) within a particular protein, e.g., where said protein is associated with a pathogen. Such evaluation is then used in the development of antibodies, entry inhibitors, vaccines, and/or other therapeutics for treating, preventing, or ameliorating disease caused by the pathogen. For example, in certain embodiments, methods presented herein are used to evaluate a SARS-CoV2 spike (S) protein or a receptor-binding domain (RBD) thereof that binds to receptors on SARS-CoV2 host cells, such as human or bat angiotensin-converting enzyme 2 (ACE2) receptors, to facilitate infection of host cells, or a nucleic acid sequence encoding the same. Thus, for example, the present specification includes use of computer-implemented methods provided herein for analysis of a SARS-CoV2 spike (S) protein or a RBD thereof to identify sequences useful in development of antibodies, entry inhibitors, vaccines, and/or other therapeutics to treat, prevent, or ameliorate the disease caused by the SARS-CoV2 virus, i.e., COVID-19.

In certain embodiments, methods presented herein are used to evaluate coding sequences of a nucleic acid that encodes a SARS-CoV2 spike (S) protein or a receptor-binding domain (RBD) thereof, conserved sequences of a nucleic acid sequence that encodes a SARS-CoV2 spike (S) protein or a RBD thereof, non-conserved domains (sequences characterized by variation) of a nucleic acid that encodes a SARS-CoV2 spike (S) protein or a RBD thereof, conserved domains of a particular SARS-CoV2 spike (S) protein or a RBD thereof, and/or non-conserved domains (sections characterized by variation) of a SARS-CoV2 spike (S) protein or a RBD thereof. In certain embodiments, methods presented herein are used to evaluate coding sequences of a nucleic acid that encodes a coronavirus spike protein (e.g., a MERS or SARS-CoV spike protein) or a RBD thereof, conserved sequences of a nucleic acid sequence that encodes a coronavirus spike protein (e.g., a MERS or SARS-CoV spike protein) or a RBD thereof, non-conserved sequences (sequences characterized by variation) of a nucleic acid that encodes a coronavirus spike protein (e.g., a MERS or SARS-CoV spike protein) or a RBD thereof, conserved domains of a particular coronavirus spike protein (e.g., a MERS or SARS-CoV spike protein) or a RBD thereof, and/or non-conserved domains (sections characterized by variation) of a coronavirus spike protein (e.g., a MERS or SARS-CoV spike protein) or a RBD thereof.

Identification of Candidate Vaccine Antigens

Vaccines include non-pathogenic substances administered to stimulate recipient production of antibodies against a pathogen (vaccine antigens). A vaccine antigen can be a peptide that is presented by the pathogen. Vaccine efficacy requires that the antibodies produced by the recipient in response to the vaccine antigen are capable of binding the pathogen if the recipient is later infected. Because strains of a pathogen can differ, vaccines provide immunity against the broadest range of pathogen strains when the vaccine antigen has or is encoded by a conserved sequence. As is disclosed herein with respect to identification of antigens for selection of anti-antigen antibodies, methods and systems of the present disclosure can be used to identify conserved pathogen sequences. Accordingly, conserved pathogen sequences identified using methods and systems of the present disclosure can be utilized as vaccine antigens and/or candidate vaccine antigens. Candidate vaccine antigens can be validated in clinically appropriate animal models of immunization and infection, and further validated in clinical trials, e.g., for safety and efficacy.

Identification of Representative Samples

Although many strains of various pathogens are known or likely to exist in clinical samples, research often focuses on one or a few strains for practical and/or historical reasons. However, in the development of therapeutics, use of research strains that are representative of clinical samples, preferably of many or most clinical samples, of the pathogen facilitates discovery of therapeutics with broad clinical efficacy. The present disclosure provides methods and systems that can be used for comparison of sequences of one or more research strains with diverse collections of sequences from other strains (e.g., diverse clinical isolates) to characterize conservation of the genome of the one or more research strains as compared to others. Conservation of sequences of research strains indicates that an analyzed research strain, or research strain sequence, is representative of all or a substantial number of compared strains. Accordingly, research strains, or research strain sequences, that demonstrate conservation in analysis according to methods and systems of the present disclosure are suitable for clinically relevant research. By contrast, research strains, or research strain sequences, that do not demonstrate conservation in analysis according to methods and systems of the present disclosure may not be optimal for clinically relevant research.

Identification of Antibiotic Resistance Markers

Antibiotic resistance of pathogenic bacteria a subject of growing clinical concern. For instance, resistant infections are much more likely to result in mortality. Bacteria acquire resistance to antibiotics through two principal routes: chromosomal mutation and the acquisition of mobile genetic elements such as plasmids by horizontal gene transfer. Plasmids are extra-genomic circular DNA molecules that replicate independently of the chromosome and are able to transfer horizontally between bacteria by conjugation. Thus, plasmids play an important role in the dissemination of antibiotic resistance in many pathogens.

Methods and systems provided herein can be applied to identify genetic and/or amino acid sequences indicative and/or causal of antibody resistance of pathogenic bacteria (antibody resistance markers). Methods and systems provided herein can be applied to plasmid sequences to identify conserved sequences. Conserved sequences of plasmids are therefore identified as candidate antibiotic resistance markers. Moreover, conserved sequences of plasmids are candidate targets for development of therapeutic agents that disrupt or neutralize plasmid-conferred antibiotic resistance.

Generation of Peptide Discovery Resources for Mass Spectrometry

Mass spectrometry identifies analyzed substances based on their precisely measured mass-to-charge ratio. Peptide mass-to-charge ratios are dependent upon peptide sequence. At least in part because mass-to-charge ratios are complex, a mass spectrometry analysis may identify peptides by comparing detected mass-to-charge ratios against a collection of expected mass-to-charge ratios. As a result, mass spectrometry can fail to identify unexpected sequences. Because organisms of a particular species, e.g., clinically relevant isolates of pathogens, vary in their genomes and proteomes, analysis of diverse samples can be hindered by an inability to identify unexpected peptides.

Methods and systems of the present disclosure can provide peptide discovery resources for mass spectrometry by analyzing the conservation characteristics of diverse genomes representative of a species of interest, e.g., of a clinically relevant pathogen. For instance, analysis according to methods and systems of the present disclosure can identify regions of sequence diversity that can be used to revise the collection of expected mass-to-charge ratios used to query mass spectrometry data. Thus, incorporation of diverse sequences identified by methods and systems of the present disclosure can enhance the power of mass spectrometry to discover peptides in samples, e.g., to discovery clinically relevant pathogen peptides.

To provide one particular example, major histocompatibility complex I associated proteins are of clinical relevance and can be discovered by mass spectrometry, provided data are analyzed based on an appropriate collection of expected mass-to-charge ratios. Major histocompatibility complexes (MHCs or HLAs in humans) are expressed on the cell surface of all nucleated cells and act as the machinery for antigen presentation to T cells in the acquired immune system. They function to display peptide fragments of processed self and foreign proteins (antigens) on the cell surface for inspection by T lymphocytes (CD8⁺ cytotoxic T lymphocytes (CTL) for MHC Class I, and CD4⁺ helper T lymphocytes for MHC Class II). Characterizing antigens involved in this process contributes to identification of therapeutically useful targets, e.g., as antigens for development of therapeutic antibodies. Mass spectrometry is a technique that can be used to identify MHC-presented antigens. However, MHC-presented antigens cannot be detected if the mass spectrometry analysis is not designed to detect the antigens present. Methods and systems disclosed herein can be used to generate an inclusive collection of expected mass-to-charge ratios to query mass spectrometry data for MHC-presented antigens of a target pathogen.

Identification of Regions of Diversity within Genomes, Genes, and Proteins (e.g., Antigens)

As disclosed herein, provided methods and systems can be used to identify regions of diversity within genomes, genes and proteins. Regions of diversity (regions that are less conserved than others) can indicate nucleotide or amino acid positions that may be amenable to more substantial laboratory manipulation, e.g., to laboratory-introduced sequence modifications. In certain biological contexts, the character of sequence diversity is critical to biological function, as is the case for example in the variable regions of immunoglobulins. Diversity can also indicate regions that may be useful for phylogenetic analyses, as regions of diversity can provide a larger number of sequence variations for phylogenetic analysis over a same or shorter period of time as compared to analysis of a relatively more conserved sequence. Diversity can also be indicative of sequences subject to evolutionary development more recently than conserved sequences.

Generation of Phylogenies of Epidemy-Causing Pathogens

Methods and systems disclosed herein can be used to generate phylogenies. Phylogenies are particularly useful for the analysis of sequences from pathogens, e.g., rapidly evolving pathogens. Phylogenies can be used to describe the molecular epidemiology and transmission of pathogens such as the human immunodeficiency virus (HIV), the origins and subsequent evolution of a severe acute respiratory syndrome (SARS)-associated coronavirus (e.g., Severe Acute Respiratory Syndrome-associated coronavirus (SARS-CoV); Severe Acute Respiratory Syndrome coronavirus 2 (SARS-CoV2), which is the virus that causes the coronavirus disease (COVID-19), Middle East Respiratory Syndrome-associated coronavirus (MERS-CoV), the evolving epidemiology of avian influenza, and seasonal and pandemic human influenza viruses. Examples of information that can be determined using phylogenies include estimations (with confidence limits) of the actual time of the origin of a new pathogen strain or its emergence in a new species, pathogen recombination and reassortment events, the rate of population size change in a pathogen epidemic, and how the pathogen spreads and evolves within a specific population and geographical region.

Genomic studies have confirmed that mutations and acquisition of mobile genetic elements can dramatically impact the pathology of microbial clones. Indeed, even a modest genetic change can have a dramatic impact on host-pathogen interaction, as well as antibody recognition of the pathogen. Within-host evolution has implications not only for patients, but also for establishing thresholds to differentiate relatedness in strains for epidemiological purposes in hospitals. Microbial genetic diversity, immunomodulation, and damage by individual strains can vary dramatically. Thus, programs that capture the breadth of clones to account for the diversity in host-pathogen interactions at the genomic level will likely yield unique understanding of the biology of microbial pathogen. That understanding promotes the development of more effective and personalized approaches for preventing infection and improving management of pathogens.

Sequence-derived information obtained from phylogenies can assist in the design and implementation of public health and therapeutic interventions. For example, as applied to HBV, methods and systems of the present disclosure could be used to determine which HBV lineage a particular strain (e.g., a laboratory strain) belongs to, determine the genetic diversity of one or more HBV genes or proteins (e.g., HBsAg) across HBV lineages, determine the number and breadth of genetic variants of HBV or of an HBV gene or protein (e.g., HBsAg) that exist in nature, and/or determine what portion of the HBV genome or of a genetic or encoded protein sequence thereof (e.g., of HBsAg) is generically conserved. In another example, methods and systems disclosed herein could be used to determine what strain with which a particular patient is infected and/or the defining genetic characteristics of such a strain and/or the antibiotic resistance characteristics of a strain with which a particular patient is infected. In another example, methods and systems disclosed herein could be used to determine the genetic diversity of a pathogen genome, e.g., the Ebola genome, and determine whether measured variations have clinical ramifications.

Identification of Orthologous Genes

Orthologs are homologous sequences of different species that descend from a common ancestral DNA sequence. Comparative genetics among species is based at least in part on the fact that orthologs are thought to be functionally related between species. Although detailed analysis can often establish the accuracy of ortholog identification, bulk analysis of genomic information has increased the rate of error in ortholog identification. Accordingly, improved methods of distinguishing real from mis-annotated orthologs are needed. As disclosed herein, methods and systems of the present disclosure can be used to characterize sequence conservation. Accordingly, methods and systems of the present disclosure can be used to improve the accuracy of ortholog identification, and/or to identify and correct existing ortholog mis-annotations. Identification of orthologs according to methods and systems disclosed herein can be used to annotate new or uncharacterized sequences by aligning the new or uncharacterized sequences with previously annotated sequences and applying the previous annotations to orthologous new or uncharacterized sequences.

Evaluation of Epitope Sequence Variation for Selection of Antibody Therapies, Identification of Putative Escape Mutants, and Personalized Medicine

In various embodiments, it is useful to evaluate variation in a particular gene or protein, or a portion thereof. For example, in the context of antibody therapy, a number of important questions can be addressed by evaluation of variation in the antigen and/or epitope of an antibody.

Various embodiments of the present specification include a therapy and/or therapeutic agent. In various embodiments, a therapy and/or therapeutic agent can be or include a small interfering RNA (siRNA) or short hairpin RNA (shRNA). In various embodiments, a therapy and/or therapeutic agent can be or include an antibody. In various embodiments, a therapy and/or therapeutic agent can be or include a therapy and/or therapeutic agent that treats COVID-19. Exemplary therapies and/or therapeutic agents that treat COVID-19 can include remdesivir, kaletra, ivermectin, tamiflu, avigan, colcrys, dexamethasone, chloroquine, hydroxychloroquine, azithromycin, it-6 inhibitors (e.g., tocilizumab and sarilumab), kinase inhibitors (e.g., acalabrutinib, ibrutinib, zanubrutinib, baricitinib, ruxolitinib, and tofacitinib), interferons, convalescent plasma, antibodies that bind SARS-CoV-2 spike protein (anti-SARS-CoV-2-Spike protein antibodies), mAb10933 (Regeneron), mAb10934 (Regeneron), mAb10987 (Regeneron), mAb10989 (Regeneron), REGN-COV2 (Regeneron), LY-CoV555 (Eli Lilly), LY-CoV016 (Eli Lilly), and/or BNT162b2 (Pfizer). Exemplary antibodies can include antibodies that bind the spike protein of SARS-CoV-2 for use in COVID-19 therapy, e.g., as disclosed in U.S. Pat. No. 10,787,501, which is incorporated herein by reference in its entirety and particularly with respect to COVID-19 therapeutic antibodies as well as their epitopes and other properties. Table 1 of U.S. Pat. No. 10,787,501, which provides exemplary anti-SARS-CoV-2-Spike protein (SARS-CoV-2-S) antibodies and antibody sequences, is specifically incorporated by reference in its entirety. See also Table 3 below:

TABLE 3

| Antibody | Component | | SEQ |
|---|---|---|---|
| Designation | Part | Sequence | ID NO |
| | Amino Acids | | |
| mAb10933 | HCVR | QVQLVESGGGLVKPGGSLRLSCAASGFTFSDYYM | 29 |
| | | SWIRQAPGKGLEWVSYITYSGSTIYYADSVKGRF | |
| | | TISRDNAKSSLYLQMNSLRAEDTAVYYCARDRGT | |
| | | TMVPFDYWGQGTLVTVSS | |
| | HCDR1 | GFTFSDYY | 30 |
| | HCDR2 | ITYSGSTI | 31 |
| | HCDR3 | ARDRGTTMVPFDY | 32 |
| | LCVR | DIQMTQSPSSLSASVGDRVTITCQASQDITNYLN | 33 |
| | | WYQQKPGKAPKLLIYAASNLETGVPSRFSGSGSG | |
| | | TDFTFTISGLQPEDIATYYCQQYDNLPLTFGGGT | |
| | | KVEIK | |
| | LCDR1 | QDITNY | 34 |
| | LCDR2 | AAS | 35 |
| | LCDR3 | QQYDNLPLT | 36 |
| | HC | QVQLVESGGGLVKPGGSLRLSCAASGFTFSDYYM | 37 |
| | | SWIRQAPGKGLEWVSYITYSGSTIYYADSVKGRF | |
| | | TISRDNAKSSLYLQMNSLRAEDTAVYYCARDRGT | |
| | | TMVPFDYWGQGTLVTVSSASTKGPSVFPLAPSSK | |
| | | STSGGTAALGCLVKDYFPEPVTVSWNSGALTSGV | |
| | | HTFPAVLQSSGLYSLSSVVTVPSSSLGTQTYICN | |
| | | VNHKPSNTKVDKKVEPKSCDKTHTCPPCPAPELL | |
| | | GGPSVFLFPPKPKDTLMISRTPEVTCVVVDVSHE | |
| | | DPEVKFNWYVDGVEVHNAKTKPREEQYNSTYRVV | |
| | | SVLTVLHQDWLNGKEYKCKVSNKALPAPIEKTIS | |
| | | KAKGQPREPQVYTLPPSRDELTKNQVSLTCLVKG | |
| | | FYPSDIAVEWESNGQPENNYKTTPPVLDSDGSFF | |
| | | LYSKLTVDKSRWQQGNVFSCSVMHEALHNHYTQK | |
| | | SLSLSPGK | |
| | LC | DIQMTQSPSSLSASVGDRVTITCQASQDITNYLN | 38 |
| | | WYQQKPGKAPKLLIYAASNLETGVPSRFSGSGSG | |
| | | TDFTFTISGLQPEDIATYYCQQYDNLPLTFGGGT | |
| | | KVEIKRTVAAPSVFIFPPSDEQLKSGTASVVCLL | |
| | | NNFYPREAKVQWKVDNALQSGNSQESVTEQDSKD | |
| | | STYSLSSTLTLSKADYEKHKVYACEVTHQGLSSP | |
| | | VTKSFNRGEC | |
| | Nucleic Acids | | |
| | HCVR | CAGGTGCAGCTGGTGGAGTCTGGGGGAGGCTTGG | 39 |
| | | TCAAGCCTGGAGGGTCCCTGAGACTCTCCTGTGC | |
| | | AGCCTCTGGATTCACCTTCAGTGACTACTACATG | |
| | | AGCTGGATCCGCCAGGCTCCAGGGAAGGGGCTGG | |
| | | AGTGGGTTTCATACATTACTTATAGTGGTAGTAC | |
| | | CATATACTACGCAGACTCTGTGAAGGGCCGATTC | |
| | | ACCATCTCCAGGGACAACGCCAAGAGCTCACTGT | |
| | | ATCTGCAAATGAACAGCCTGAGAGCCGAGGACAC | |
| | | GGCCGTGTATTACTGTGCGAGAGATCGCGGTACA | |
| | | ACTATGGTCCCCTTTGACTACTGGGGCCAGGGAA | |
| | | CCCTGGTCACCGTCTCCTCA | |
| | HCDR1 | GGATTCACCTTCAGTGACTACTAC | 40 |
| | HCDR2 | ATTACTTATAGTGGTAGTACCATA | 41 |
| | HCDR3 | GCGAGAGATCGCGGTACAACTATGGTCCCCTTTG | 42 |
| | | ACTAC | |
| | LCVR | GACATCCAGATGACCCAGTCTCCATCCTCCCTGT | 43 |
| | | CTGCATCTGTAGGAGACAGAGTCACCATCACTTG | |
| | | CCAGGCGAGTCAGGACATTACCAACTATTTAAAT | |
| | | TGGTATCAGCAGAAACCAGGGAAAGCCCCTAAGC | |
| | | TCCTGATCTACGCTGCATCCAATTTGGAAACAGG | |
| | | GGTCCCATCAAGGTTCAGTGGAAGTGGATCTGGG | |
| | | ACAGATTTTACTTTCACCATCAGCGGCCTGCAGC | |
| | | CTGAAGATATTGCAACATATTACTGTCAACAGTA | |
| | | TGATAATCTCCCTCTCACTTTCGGCGGAGGGACC | |
| | | AAGGTGGAGATCAAA | |
| | LCDR1 | CAGGACATTACCAACTAT | 44 |
| | LCDR2 | GCTGCATCC | 45 |
| | LCDR3 | CAACAGTATGATAATCTCCCTCTCACT | 46 |
| | HC | CAGGTGCAGCTGGTGGAGTCTGGGGGAGGCTTGG | 47 |
| | | TCAAGCCTGGAGGGTCCCTGAGACTCTCCTGTGC | |
| | | AGCCTCTGGATTCACCTTCAGTGACTACTACATG | |
| | | AGCTGGATCCGCCAGGCTCCAGGGAAGGGGCTGG | |
| | | AGTGGGTTTCATACATTACTTATAGTGGTAGTAC | |
| | | CATATACTACGCAGACTCTGTGAAGGGCCGATTC | |
| | | ACCATCTCCAGGGACAACGCCAAGAGCTCACTGT | |
| | | ATCTGCAAATGAACAGCCTGAGAGCCGAGGACAC | |
| | | GGCCGTGTATTACTGTGCGAGAGATCGCGGTACA | |
| | | ACTATGGTCCCCTTTGACTACTGGGGCCAGGGAA | |
| | | CCCTGGTCACCGTCTCCTCAGCCTCCACCAAGGG | |
| | | CCCATCGGTCTTCCCCCTGGCACCCTCCTCCAAG | |
| | | AGCACCTCTGGGGGCACAGCGGCCCTGGGCTGCC | |
| | | TGGTCAAGGACTACTTCCCCGAACCGGTGACGGT | |
| | | GTCGTGGAACTCAGGCGCCCTGACCAGCGGCGTG | |
| | | CACACCTTCCCGGCTGTCCTACAGTCCTCAGGAC | |
| | | TCTACTCCCTCAGCAGCGTGGTGACCGTGCCCTC | |
| | | CAGCAGCTTGGGCACCCAGACCTACATCTGCAAC | |
| | | GTGAATCACAAGCCCAGCAACACCAAGGTGGACA | |
| | | AGAAAGTTGAGCCCAAATCTTGTGACAAAACTCA | |
| | | CACATGCCCACCGTGCCCAGCACCTGAACTCCTG | |
| | | GGGGGACCGTCAGTCTTCCTCTTCCCCCCAAAAC | |
| | | CCAAGGACACCCTCATGATCTCCCGGACCCCTGA | |
| | | GGTCACATGCGTGGTGGTGGACGTGAGCCACGAA | |
| | | GACCCTGAGGTCAAGTTCAACTGGTACGTGGACG | |
| | | GCGTGGAGGTGCATAATGCCAAGACAAAGCCGCG | |
| | | GGAGGAGCAGTACAACAGCACGTACCGTGTGGTC | |
| | | AGCGTCCTCACCGTCCTGCACCAGGACTGGCTGA | |
| | | ATGGCAAGGAGTACAAGTGCAAGGTCTCCAACAA | |
| | | AGCCCTCCCAGCCCCCATCGAGAAAACCATCTCC | |
| | | AAAGCCAAAGGGCAGCCCCGAGAACCACAGGTGT | |
| | | ACACCCTGCCCCCATCCCGGGATGAGCTGACCAA | |
| | | GAACCAGGTCAGCCTGACCTGCCTGGTCAAAGGC | |
| | | TTCTATCCCAGCGACATCGCCGTGGAGTGGGAGA | |
| | | GCAATGGGCAGCCGGAGAACAACTACAAGACCAC | |
| | | GCCTCCCGTGCTGGACTCCGACGGCTCCTTCTTC | |
| | | CTCTACAGCAAGCTCACCGTGGACAAGAGCAGGT | |
| | | GGCAGCAGGGGAACGTCTTCTCATGCTCCGTGAT | |
| | | GCATGAGGCTCTGCACAACCACTACACGCAGAAG | |
| | | TCCCTCTCCCTGTCTCCGGGTAAATGA | |
| | LC | GACATCCAGATGACCCAGTCTCCATCCTCCCTGT | 48 |
| | | CTGCATCTGTAGGAGACAGAGTCACCATCACTTG | |
| | | CCAGGCGAGTCAGGACATTACCAACTATTTAAAT | |
| | | TGGTATCAGCAGAAACCAGGGAAAGCCCCTAAGC | |
| | | TCCTGATCTACGCTGCATCCAATTTGGAAACAGG | |
| | | GGTCCCATCAAGGTTCAGTGGAAGTGGATCTGGG | |
| | | ACAGATTTTACTTTCACCATCAGCGGCCTGCAGC | |
| | | CTGAAGATATTGCAACATATTACTGTCAACAGTA | |
| | | TGATAATCTCCCTCTCACTTTCGGCGGAGGGACC | |
| | | AAGGTGGAGATCAAACGAACTGTGGCTGCACCAT | |
| | | CTGTCTTCATCTTCCCGCCATCTGATGAGCAGTT | |
| | | GAAATCTGGAACTGCCTCTGTTGTGTGCCTGCTG | |
| | | AATAACTTCTATCCCAGAGAGGCCAAAGTACAGT | |
| | | GGAAGGTGGATAACGCCCTCCAATCGGGTAACTC | |
| | | CCAGGAGAGTGTCACAGAGCAGGACAGCAAGGAC | |
| | | AGCACCTACAGCCTCAGCAGCACCCTGACGCTGA | |
| | | GCAAAGCAGACTACGAGAAACACAAAGTCTACGC | |
| | | CTGCGAAGTCACCCATCAGGGCCTGAGCTCGCCC | |
| | | GTCACAAAGAGCTTCAACAGGGGAGAGTGTTAG | |
| | Amino Acids | | |
| mAb10934 | HCVR | EVQLVESGGGLVKPGGSLRLSCAASGITFSNAWM | 49 |
| | | SWVRQAPGKGLEWVGRIKSKTDGGTTDYAAPVKG | |
| | | RFTISRDDSKNTLYLQMNSLKTEDTAVYYCTTAR | |
| | | WDWYFDLWGRGTLVTVSS | |
| | HCDR1 | GITFSNAW | 50 |
| | HCDR2 | IKSKTDGGTT | 51 |
| | HCDR3 | TTARWDWYFDL | 52 |
| | LCVR | DIQMTQSPSSLSASVGDRVTITCQASQDIWNYIN | 53 |
| | | WYQQKPGKAPKLLIYDASNLKTGVPSRFSGSGSG | |
| | | TDFTFTISSLQPEDIATYYCQQHDDLPPTFGQGT | |
| | | KVEIK | |
| | LCDR1 | QDIWNY | 54 |
| | LCDR2 | DAS | 55 |
| | LCDR3 | QQHDDLPPT | 56 |
| | HC | EVQLVESGGGLVKPGGSLRLSCAASGITFSNAWM | 57 |
| | | SWVRQAPGKGLEWVGRIKSKTDGGTTDYAAPVKG | |
| | | RFTISRDDSKNTLYLQMNSLKTEDTAVYYCTTAR | |
| | | WDWYFDLWGRGTLVTVSSASTKGPSVFPLAPSSK | |
| | | STSGGTAALGCLVKDYFPEPVTVSWNSGALTSGV | |
| | | HTFPAVLQSSGLYSLSSVVTVPSSSLGTQTYICN | |
| | | VNHKPSNTKVDKKVEPKSCDKTHTCPPCPAPELL | |
| | | GGPSVFLFPPKPKDTLMISRTPEVTCVVVDVSHE | |
| | | DPEVKFNWYVDGVEVHNAKTKPREEQYNSTYRVV | |
| | | SVLTVLHQDWLNGKEYKCKVSNKALPAPIEKTIS | |
| | | KAKGQPREPQVYTLPPSRDELTKNQVSLTCLVKG | |
| | | FYPSDIAVEWESNGQPENNYKTTPPVLDSDGSFF | |
| | | LYSKLTVDKSRWQQGNVFSCSVMHEALHNHYTQK | |
| | | SLSLSPGK | |
| | LC | DIQMTQSPSSLSASVGDRVTITCQASQDIWNYIN | 58 |
| | | WYQQKPGKAPKLLIYDASNLKTGVPSRFSGSGSG | |
| | | TDFTFTISSLQPEDIATYYCQQHDDLPPTFGQGT | |
| | | KVEIKRTVAAPSVFIFPPSDEQLKSGTASVVCLL | |
| | | NNFYPREAKVQWKVDNALQSGNSQESVTEQDSKD | |
| | | STYSLSSTLTLSKADYEKHKVYACEVTHQGLSSP | |
| | | VTKSFNRGEC | |
| | Nucleic Acids | | |
| | HCVR | GAGGTGCAGCTGGTGGAGTCTGGGGGAGGCTTGG | 59 |
| | | TAAAGCCTGGGGGGTCCCTTAGACTCTCCTGTGC | |
| | | AGCCTCTGGAATCACTTTCAGTAACGCCTGGATG | |
| | | AGTTGGGTCCGCCAGGCTCCAGGGAAGGGGCTGG | |
| | | AGTGGGTTGGCCGTATTAAAAGCAAAACTGATGG | |
| | | TGGGACAACAGACTACGCCGCACCCGTGAAAGGC | |
| | | AGATTCACCATCTCAAGAGATGATTCAAAAAACA | |
| | | CGCTGTATCTACAAATGAACAGCCTGAAAACCGA | |
| | | GGACACAGCCGTGTATTACTGTACCACAGCGAGG | |
| | | TGGGACTGGTACTTCGATCTCTGGGGCCGTGGCA | |
| | | CCCTGGTCACTGTCTCCTCA | |
| | HCDR1 | GGAATCACTTTCAGTAACGCCTGG | 60 |
| | HCDR2 | ATTAAAAGCAAAACTGATGGTGGGACAACA | 61 |
| | HCDR3 | ACCACAGCGAGGTGGGACTGGTACTTCGATCTC | 62 |
| | LCVR | GACATCCAGATGACCCAGTCTCCATCCTCCCTGT | 63 |
| | | CTGCATCTGTAGGAGACAGAGTCACCATCACTTG | |
| | | CCAGGCGAGTCAGGACATTTGGAATTATATAAAT | |
| | | TGGTATCAGCAGAAACCAGGGAAGGCCCCTAAGC | |
| | | TCCTGATCTACGATGCATCCAATTTGAAAACAGG | |
| | | GGTCCCATCAAGGTTCAGTGGAAGTGGATCTGGG | |
| | | ACAGATTTTACTTTCACCATCAGCAGCCTGCAGC | |
| | | CTGAAGATATTGCAACATATTACTGTCAACAGCA | |
| | | TGATGATCTCCCTCCGACCTTCGGCCAAGGGACC | |
| | | AAGGTGGAAATCAA | |
| | LCDR1 | CAGGACATTTGGAATTAT | 64 |
| | LCDR2 | GATGCATCC | 65 |
| | LCDR3 | CAACAGCATGATGATCTCCCTCCGACC | 66 |
| | HC | GAGGTGCAGCTGGTGGAGTCTGGGGGAGGCTTGG | 67 |
| | | TAAAGCCTGGGGGGTCCCTTAGACTCTCCTGTGC | |
| | | AGCCTCTGGAATCACTTTCAGTAACGCCTGGATG | |
| | | AGTTGGGTCCGCCAGGCTCCAGGGAAGGGGCTGG | |
| | | AGTGGGTTGGCCGTATTAAAAGCAAAACTGATGG | |
| | | TGGGACAACAGACTACGCCGCACCCGTGAAAGGC | |
| | | AGATTCACCATCTCAAGAGATGATTCAAAAAACA | |
| | | CGCTGTATCTACAAATGAACAGCCTGAAAACCGA | |
| | | GGACACAGCCGTGTATTACTGTACCACAGCGAGG | |
| | | TGGGACTGGTACTTCGATCTCTGGGGCCGTGGCA | |
| | | CCCTGGTCACTGTCTCCTCAGCCTCCACCAAGGG | |
| | | CCCATCGGTCTTCCCCCTGGCACCCTCCTCCAAG | |
| | | AGCACCTCTGGGGGCACAGCGGCCCTGGGCTGCC | |
| | | TGGTCAAGGACTACTTCCCCGAACCGGTGACGGT | |
| | | GTCGTGGAACTCAGGCGCCCTGACCAGCGGCGTG | |
| | | CACACCTTCCCGGCTGTCCTACAGTCCTCAGGAC | |
| | | TCTACTCCCTCAGCAGCGTGGTGACCGTGCCCTC | |
| | | CAGCAGCTTGGGCACCCAGACCTACATCTGCAAC | |
| | | GTGAATCACAAGCCCAGCAACACCAAGGTGGACA | |
| | | AGAAAGTTGAGCCCAAATCTTGTGACAAAACTCA | |
| | | CACATGCCCACCGTGCCCAGCACCTGAACTCCTG | |
| | | GGGGGACCGTCAGTCTTCCTCTTCCCCCCAAAAC | |
| | | CCAAGGACACCCTCATGATCTCCCGGACCCCTGA | |
| | | GGTCACATGCGTGGTGGTGGACGTGAGCCACGAA | |
| | | GACCCTGAGGTCAAGTTCAACTGGTACGTGGACG | |
| | | GCGTGGAGGTGCATAATGCCAAGACAAAGCCGCG | |
| | | GGAGGAGCAGTACAACAGCACGTACCGTGTGGTC | |
| | | AGCGTCCTCACCGTCCTGCACCAGGACTGGCTGA | |
| | | ATGGCAAGGAGTACAAGTGCAAGGTCTCCAACAA | |
| | | AGCCCTCCCAGCCCCCATCGAGAAAACCATCTCC | |
| | | AAAGCCAAAGGGCAGCCCCGAGAACCACAGGTGT | |
| | | ACACCCTGCCCCCATCCCGGGATGAGCTGACCAA | |
| | | GAACCAGGTCAGCCTGACCTGCCTGGTCAAAGGC | |
| | | TTCTATCCCAGCGACATCGCCGTGGAGTGGGAGA | |
| | | GCAATGGGCAGCCGGAGAACAACTACAAGACCAC | |
| | | GCCTCCCGTGCTGGACTCCGACGGCTCCTTCTTC | |
| | | CTCTACAGCAAGCTCACCGTGGACAAGAGCAGGT | |
| | | GGCAGCAGGGGAACGTCTTCTCATGCTCCGTGAT | |
| | | GCATGAGGCTCTGCACAACCACTACACGCAGAAG | |
| | | TCCCTCTCCCTGTCTCCGGGTAAATGA | |
| | LC | GACATCCAGATGACCCAGTCTCCATCCTCCCTGT | 68 |
| | | CTGCATCTGTAGGAGACAGAGTCACCATCACTTG | |
| | | CCAGGCGAGTCAGGACATTTGGAATTATATAAAT | |
| | | TGGTATCAGCAGAAACCAGGGAAGGCCCCTAAGC | |
| | | TCCTGATCTACGATGCATCCAATTTGAAAACAGG | |
| | | GGTCCCATCAAGGTTCAGTGGAAGTGGATCTGGG | |
| | | ACAGATTTTACTTTCACCATCAGCAGCCTGCAGC | |
| | | CTGAAGATATTGCAACATATTACTGTCAACAGCA | |
| | | TGATGATCTCCCTCCGACCTTCGGCCAAGGGACC | |
| | | AAGGTGGAAATCAAACGAACTGTGGCTGCACCAT | |
| | | CTGTCTTCATCTTCCCGCCATCTGATGAGCAGTT | |
| | | GAAATCTGGAACTGCCTCTGTTGTGTGCCTGCTG | |
| | | AATAACTTCTATCCCAGAGAGGCCAAAGTACAGT | |
| | | GGAAGGTGGATAACGCCCTCCAATCGGGTAACTC | |
| | | CCAGGAGAGTGTCACAGAGCAGGACAGCAAGGAC | |
| | | AGCACCTACAGCCTCAGCAGCACCCTGACGCTGA | |
| | | GCAAAGCAGACTACGAGAAACACAAAGTCTACGC | |
| | | CTGCGAAGTCACCCATCAGGGCCTGAGCTCGCCC | |
| | | GTCACAAAGAGCTTCAACAGGGGAGAGTGTTAG | |
| | Amino Acids | | |
| mAb10987 | HCVR | QVQLVESGGGVVQPGRSLRLSCAASGFTFSNYAM | 69 |
| | | YWVRQAPGKGLEWVAVISYDGSNKYYADSVKGRF | |
| | | TISRDNSKNTLYLQMNSLRTEDTAVYYCASGSDY | |
| | | GDYLLVYWGQGTLVTVSS | |
| | HCDR1 | GFTFSNYA | 70 |
| | HCDR2 | ISYDGSNK | 71 |
| | HCDR3 | ASGSDYGDYLLVY | 72 |
| | LCVR | QSALTQPASVSGSPGQSITISCTGTSSDVGGYNY | 73 |
| | | VSWYQQHPGKAPKLMIYDVSKRPSGVSNRFSGSK | |
| | | SGNTASLTISGLQSEDEADYYCNSLTSISTWVFG | |
| | | GGTKLTVL | |
| | LCDR1 | SSDVGGYNY | 74 |
| | LCDR2 | DVS | 75 |
| | LCDR3 | NSLTSISTWV | 76 |
| | HC | QVQLVESGGGVVQPGRSLRLSCAASGFTFSNYAM | 77 |
| | | YWVRQAPGKGLEWVAVISYDGSNKYYADSVKGRF | |
| | | TISRDNSKNTLYLQMNSLRTEDTAVYYCASGSDY | |
| | | GDYLLVYWGQGTLVTVSSASTKGPSVFPLAPSSK | |
| | | STSGGTAALGCLVKDYFPEPVTVSWNSGALTSGV | |
| | | HTFPAVLQSSGLYSLSSVVTVPSSSLGTQTYICN | |
| | | VNHKPSNTKVDKKVEPKSCDKTHTCPPCPAPELL | |
| | | GGPSVFLFPPKPKDTLMISRTPEVTCVVVDVSHE | |
| | | DPEVKFNWYVDGVEVHNAKTKPREEQYNSTYRVV | |
| | | SVLTVLHQDWLNGKEYKCKVSNKALPAPIEKTIS | |
| | | KAKGQPREPQVYTLPPSRDELTKNQVSLTCLVKG | |
| | | FYPSDIAVEWESNGQPENNYKTTPPVLDSDGSFF | |
| | | LYSKLTVDKSRWQQGNVFSCSVMHEALHNHYTQK | |
| | | SLSLSPGK | |
| | LC | QSALTQPASVSGSPGQSITISCTGTSSDVGGYNY | 78 |
| | | VSWYQQHPGKAPKLMIYDVSKRPSGVSNRFSGSK | |
| | | SGNTASLTISGLQSEDEADYYCNSLTSISTWVFG | |
| | | GGTKLTVLGQPKAAPSVTLFPPSSEELQANKATL | |
| | | VCLISDFYPGAVTVAWKADSSPVKAGVETTTPSK | |
| | | QSNNKYAASSYLSLTPEQWKSHRSYSCQVTHEGS | |
| | | TVEKTVAPTECS | |
| | Nucleic Acids | | |
| | HCVR | CAGGTGCAGCTGGTGGAGTCTGGGGGAGGCGTGG | 79 |
| | | TCCAGCCTGGGAGGTCCCTGAGACTCTCCTGTGC | |
| | | AGCCTCTGGATTCACCTTCAGTAACTATGCTATG | |
| | | TACTGGGTCCGCCAGGCTCCAGGCAAGGGGCTGG | |
| | | AGTGGGTGGCAGTTATATCATATGATGGAAGTAA | |
| | | TAAATACTATGCAGACTCCGTGAAGGGCCGATTC | |
| | | ACCATCTCCAGAGACAATTCCAAGAACACGCTGT | |
| | | ATCTGCAAATGAACAGCCTGAGACTGAGGACAC | |
| | | GGCTGTGTATTACTGTGCGAGTGGCTCCGACTAC | |
| | | GGTGACTACTTATTGGTTTACTGGGGCCAGGGAA | |
| | | CCCTGGTCACCGTCTCCTCA | |
| | HCDR1 | GGATTCACCTTCAGTAACTATGCT | 80 |
| | HCDR2 | ATATCATATGATGGAAGTAATAAA | 81 |
| | HCDR3 | GCGAGTGGCTCCGACTACGGTGACTACTTATTGG | 82 |
| | | TTTAC | |
| | LCVR | CAGTCTGCCCTGACTCAGCCTGCCTCCGTGTCTG | 83 |
| | | GGTCTCCTGGACAGTCGATCACCATCTCCTGCAC | |
| | | TGGAACCAGCAGTGACGTTGGTGGTTATAACTAT | |
| | | GTCTCCTGGTACCAACAACACCCAGGCAAAGCCC | |
| | | CCAAACTCATGATTTATGATGTCAGTAAGCGGCC | |
| | | CTCAGGGGTTTCTAATCGCTTCTCTGGCTCCAAG | |
| | | TCTGGCAACACGGCCTCCCTGACCATCTCTGGGC | |
| | | TCCAGTCTGAGGACGAGGCTGATTATTACTGCAA | |
| | | CTCTTTGACAAGCATCAGCACTTGGGTGTTCGGC | |
| | | GGAGGGACCAAGCTGACCGTCCTA | |
| | LCDR1 | AGCAGTGACGTTGGTGGTTATAACTAT | 84 |
| | LCDR2 | GATGTCAGT | 85 |
| | LCDR3 | AACTCTTTGACAAGCATCAGCACTTGGGTG | 86 |
| | HC | CAGGTGCAGCTGGTGGAGTCTGGGGGAGGCGTGG | 87 |
| | | TCCAGCCTGGGAGGTCCCTGAGACTCTCCTGTGC | |
| | | AGCCTCTGGATTCACCTTCAGTAACTATGCTATG | |
| | | TACTGGGTCCGCCAGGCTCCAGGCAAGGGGCTGG | |
| | | AGTGGGTGGCAGTTATATCATATGATGGAAGTAA | |
| | | TAAATACTATGCAGACTCCGTGAAGGGCCGATTC | |
| | | ACCATCTCCAGAGACAATTCCAAGAACACGCTGT | |
| | | ATCTGCAAATGAACAGCCTGAGAACTGAGGACAC | |
| | | GGCTGTGTATTACTGTGCGAGTGGCTCCGACTAC | |
| | | GGTGACTACTTATTGGTTTACTGGGGCCAGGGAA | |
| | | CCCTGGTCACCGTCTCCTCAGCCTCCACCAAGGG | |
| | | CCCATCGGTCTTCCCCCTGGCACCCTCCTCCAAG | |
| | | AGCACCTCTGGGGGCACAGCGGCCCTGGGCTGCC | |
| | | TGGTCAAGGACTACTTCCCCGAACCGGTGACGGT | |
| | | GTCGTGGAACTCAGGCGCCCTGACCAGCGGCGTG | |
| | | CACACCTTCCCGGCTGTCCTACAGTCCTCAGGAC | |
| | | TCTACTCCCTCAGCAGCGTGGTGACCGTGCCCTC | |
| | | CAGCAGCTTGGGCACCCAGACCTACATCTGCAAC | |
| | | GTGATCACAAGCCCAGCAACACCAAGGTGGACA | |
| | | AGAAAGTTGAGCCCAAATCTTGTGACAAAACTCA | |
| | | CACATGCCCACCGTGCCCAGCACCTGAACTCCTG | |
| | | GGGGGACCGTCAGTCTTCCTCTTCCCCCCAAAAC | |
| | | CCAAGGACACCCTCATGATCTCCCGGACCCCTGA | |
| | | GGTCACATGCGTGGTGGTGGACGTGAGCCACGAA | |
| | | GACCCTGAGGTCAAGTTCAACTGGTACGTGGACG | |
| | | GCGTGGAGGTGCATAATGCCAAGACAAAGCCGCG | |
| | | GGAGGAGCAGTACAACAGCACGTACCGTGTGGTC | |
| | | AGCGTCCTCACCGTCCTGCACCAGGACTGGCTGA | |
| | | ATGGCAAGGAGTACAAGTGCAAGGTCTCCAACAA | |
| | | AGCCCTCCCAGCCCCCATCGAGAAAACCATCTCC | |
| | | AAAGCCAAAGGGCAGCCCCGAGAACCACAGGTGT | |
| | | ACACCCTGCCCCCATCCCGGGATGAGCTGACCAA | |
| | | GAACCAGGTCAGCCTGACCTGCCTGGTCAAAGGC | |
| | | TTCTATCCCAGCGACATCGCCGTGGAGTGGGAGA | |
| | | GCAATGGGCAGCCGGAGAACAACTACAAGACCAC | |
| | | GCCTCCCGTGCTGGACTCCGACGGCTCCTTCTTC | |
| | | CTCTACAGCAAGCTCACCGTGGACAAGAGCAGGT | |
| | | GGCAGCAGGGGAACGTCTTCTCATGCTCCGTGAT | |
| | | GCATGAGGCTCTGCACAACCACTACACGCAGAAG | |
| | | TCCCTCTCCCTGTCTCCGGGTAAATGA | |
| | LC | CAGTCTGCCCTGACTCAGCCTGCCTCCGTGTCTG | 88 |
| | | GGTCTCCTGGACAGTCGATCACCATCTCCTGCAC | |
| | | TGGAACCAGCAGTGACGTTGGTGGTTATAACTAT | |
| | | GTCTCCTGGTACCAACAACACCCAGGCAAAGCCC | |
| | | CCAAACTCATGATTTATGATGTCAGTAAGCGGCC | |
| | | CTCAGGGGTTTCTAATCGCTTCTCTGGCTCCAAG | |
| | | TCTGGCAACACGGCCTCCCTGACCATCTCTGGGC | |
| | | TCCAGTCTGAGGACGAGGCTGATTATTACTGCAA | |
| | | CTCTTTGACAAGCATCAGCACTTGGGTGTTCGGC | |
| | | GGAGGGACCAAGCTGACCGTCCTAGGCCAGCCCA | |
| | | AGGCCGCCCCCTCCGTGACCCTGTTCCCCCCCTC | |
| | | CTCCGAGGAGCTGCAGGCCAACAAGGCCACCCTG | |
| | | GTGTGCCTGATCTCCGACTTCTACCCCGGCGCCG | |
| | | TGACCGTGGCCTGGAAGGCCGACTCCTCCCCCGT | |
| | | GAAGGCCGGCGTGGAGACCACCACCCCCTCCAAG | |
| | | CAGTCCAACAACAAGTACGCCGCCTCCTCCTACC | |
| | | TGTCCCTGACCCCCGAGCAGTGGAAGTCCCACCG | |
| | | GTCCTACTCCTGCCAGGTGACCCACGAGGGCTCC | |
| | | ACCGTGGAGAAGACCGTGGCCCCCACCGAGTGCT | |
| | | CCTGA | |
| mAb10989 | Amino Acids | | |
| | HCVR | QVQLVQSGAEVKKPGASVKVSCKASGYIFTGYYM | 89 |
| | | HWVRQAPGQGLEWMGWINPNSGGANYAQKFQGRV | |
| | | TLTRDTSITTVYMELSRLRFDDTAVYYCARGSRY | |
| | | DWNQNNWFDPWGQGTLVTVSS | |
| | HCDR1 | GYIFTGYY | 90 |
| | HCDR2 | INPNSGGA | 91 |
| | HCDR3 | ARGSRYDWNQNNWFDP | 92 |
| | LCVR | QSALTQPASVSGSPGQSITISCTGTSSDVGTYNY | 93 |
| | | VSWYQQHPGKAPKLMIFDVSNRPSGVSDRFSGSK | |
| | | SGNTASLTISGLQAEDEADYYCSSFTTSSTVVFG | |
| | | GGTKLTVL | |
| | LCDR1 | SSDVGTYNY | 94 |
| | LCDR2 | DVS | 75 |
| | LCDR3 | SSFTTSSTVV | 95 |
| | HC | QVQLVQSGAEVKKPGASVKVSCKASGYIFTGYYM | 96 |
| | | HWVRQAPGQGLEWMGWINPNSGGANYAQKFQGRV | |
| | | TLTRDTSITTVYMELSRLRFDDTAVYYCARGSRY | |
| | | DWNQNNWFDPWGQGTLVTVSSASTKGPSVFPLAP | |
| | | SSKSISGGTAALGCLVKDYFPEPVTVSWNSGALT | |
| | | SGVHTFPAVLQSSGLYSLSSVVTVPSSSLGTQTY | |
| | | ICNVNHKPSNTKVDKKVEPKSCDKTHTCPPCPAP | |
| | | ELLGGPSVFLFPPKPKDTLMISRTPEVTCVVVDV | |
| | | SHEDPEVKFNWYVDGVEVHNAKTKPREEQYNSTY | |
| | | RVVSVLTVLHQDWLNGKEYKCKVSNKALPAPIEK | |
| | | TISKAKGQPREPQVYTLPPSRDELTKNQVSLTCL | |
| | | VKGFYPSDIAVEWESNGQPENNYKTTPPVLDSDG | |
| | | SFFLYSKLTVDKSRWQQGNVFSCSVMHEALHNHY | |
| | | TQKSLSLSPGK | |
| | LC | QSALTQPASVSGSPGQSITISCTGTSSDVGTYNY | 97 |
| | | VSWYQQHPGKAPKLMIFDVSNRPSGVSDRFSGSK | |
| | | SGNTASLTISGLQAEDEADYYCSSFTTSSTVVFG | |
| | | GGTKLTVLGQPKAAPSVTLFPPSSEELQANKATL | |
| | | VCLISDFYPGAVTVAWKADSSPVKAGVETTTPSK | |
| | | QSNNKYAASSYLSLTPEQWKSHRSYSCQVTHEGS | |
| | | TVEKTVAPTECS | |
| | Nucleic Acids | | |
| | HCVR | CAGGTGCAGCTGGTGCAGTCTGGGGCTGAGGTGA | 98 |
| | | AGAAGCCTGGGGCCTCAGTGAAGGTCTCCTGCAA | |
| | | GGCTTCTGGATACATCTTCACCGGCTACTATATG | |
| | | CACTGGGTGCGACAGGCCCCTGGACAGGGGCTTG | |
| | | AGTGGATGGGATGGATCAACCCTAACAGTGGTGG | |
| | | CGCAAACTATGCACAGAAGTTTCAGGGCAGGGTC | |
| | | ACCCTGACCAGGGACACGTCCATCACCACAGTCT | |
| | | ACATGGAACTGAGCAGGCTGAGATTTGACGACAC | |
| | | GGCCGTGTATTACTGTGCGAGAGGATCCCGGTAT | |
| | | GACTGGAACCAGAACAACTGGTTCGACCCCTGGG | |
| | | GCCAGGGAACCCTGGTCACCGTCTCCTCA | |
| | HCDR1 | GGATACATCTTCACCGGCTACTAT | 99 |
| | HCDR2 | ATCAACCCTAACAGTGGTGGCGCA | 100 |
| | HCDR3 | GCGAGAGGATCCCGGTATGACTGGAACCAGAACA | 101 |
| | | ACTGGTTCGACCCC | |
| | LCVR | CAGTCTGCCCTGACTCAGCCTGCCTCCGTGTCTG | 102 |
| | | GGTCTCCTGGACAGTCGATCACCATCTCCTGCAC | |
| | | TGGAACCAGCAGTGACGTTGGTACTTATAACTAT | |
| | | GTCTCCTGGTACCAACAACACCCAGGCAAAGCCC | |
| | | CCAAACTCATGATTTTTGATGTCAGTAATCGGCC | |
| | | CTCAGGGGTTTCTGATCGCTTCTCTGGCTCCAAG | |
| | | TCTGGCAACACGGCCTCCCTGACCATCTCTGGGC | |
| | | TCCAGGCTGAGGACGAGGCTGATTATTACTGCAG | |
| | | CTCATTTACAACCAGCAGCACTGTGGTTTTCGGC | |
| | | GGAGGGACCAAGCTGACCGTCCTA | |
| | LCDR1 | AGCAGTGACGTTGGTACTTATAACTAT | 103 |
| | LCDR2 | GATGTCAGT | 104 |
| | LCDR3 | AGCTCATTTACAACCAGCAGCACTGTGGTT | 105 |
| | HC | CAGGTGCAGCTGGTGCAGTCTGGGGCTGAGGTGA | 106 |
| | | AGAAGCCTGGGGCCTCAGTGAAGGTCTCCTGCAA | |
| | | GGCTTCTGGATACATCTTCACCGGCTACTATATG | |
| | | CACTGGGTGCGACAGGCCCCTGGACAGGGGCTTG | |
| | | AGTGGATGGGATGGATCAACCCTAACAGTGGTGG | |
| | | CGCAAACTATGCACAGAAGTTTCAGGGCAGGGTC | |
| | | ACCCTGACCAGGGACACGTCCATCACCACAGTCT | |
| | | ACATGGAACTGAGCAGGCTGAGATTTGACGACAC | |
| | | GGCCGTGTATTACTGTGCGAGAGGATCCCGGTAT | |
| | | GACTGGAACCAGAACAACTGGTTCGACCCCTGGG | |
| | | GCCAGGGAACCCTGGTCACCGTCTCCTCAGCCTC | |
| | | CACCAAGGGCCCATCGGTCTTCCCCCTGGCACCC | |
| | | TCCTCCAAGAGCACCTCTGGGGGCACAGCGGCCC | |
| | | TGGGCTGCCTGGTCAAGGACTACTTCCCCGAACC | |
| | | GGTGACGGTGTCGTGGAACTCAGGCGCCCTGACC | |
| | | AGCGGCGTGCACACCTTCCCGGCTGTCCTACAGT | |
| | | CCTCAGGACTCTACTCCCTCAGCAGCGTGGTGAC | |
| | | CGTGCCCTCCAGCAGCTTGGGCACCCAGACCTAC | |
| | | ATCTGCAACGTGAATCACAAGCCCAGCAACACCA | |
| | | AGGTGGACAAGAAAGTTGAGCCCAAATCTTGTGA | |
| | | CAAAACTCACACATGCCCACCGTGCCCAGCACCT | |
| | | GAACTCCTGGGGGGACCGTCAGTCTTCCTCTTCC | |
| | | CCCCAAAACCCAAGGACACCCTCATGATCTCCCG | |
| | | GACCCCTGAGGTCACATGCGTGGTGGTGGACGTG | |
| | | AGCCACGAAGACCCTGAGGTCAAGTTCAACTGGT | |
| | | ACGTGGACGGCGTGGAGGTGCATAATGCCAAGAC | |
| | | AAAGCCGCGGGAGGAGCAGTACAACAGCACGTAC | |
| | | CGTGTGGTCAGCGTCCTCACCGTCCTGCACCAGG | |
| | | ACTGGCTGAATGGCAAGGAGTACAAGTGCAAGGT | |
| | | CTCCAACAAAGCCCTCCCAGCCCCCATCGAGAAA | |
| | | ACCATCTCCAAAGCCAAAGGGCAGCCCCGAGAAC | |
| | | CACAGGTGTACACCCTGCCCCCATCCCGGGATGA | |
| | | GCTGACCAAGAACCAGGTCAGCCTGACCTGCCTG | |
| | | GTCAAAGGCTTCTATCCCAGCGACATCGCCGTGG | |
| | | AGTGGGAGAGCAATGGGCAGCCGGAGAACAACTA | |
| | | CAAGACCACGCCTCCCGTGCTGGACTCCGACGGC | |
| | | TCCTTCTTCCTCTACAGCAAGCTCACCGTGGACA | |
| | | AGAGCAGGTGGCAGCAGGGGAACGTCTTCTCATG | |
| | | CTCCGTGATGCATGAGGCTCTGCACAACCACTAC | |
| | | ACGCAGAAGTCCCTCTCCCTGTCTCCGGGTAAAT | |
| | | GA | |
| | LC | CAGTCTGCCCTGACTCAGCCTGCCTCCGTGTCTG | 107 |
| | | GGTCTCCTGGACAGTCGATCACCATCTCCTGCAC | |
| | | TGGAACCAGCAGTGACGTTGGTACTTATAACTAT | |
| | | GTCTCCTGGTACCAACAACACCCAGGCAAAGCCC | |
| | | CCAAACTCATGATTTTTGATGTCAGTAATCGGCC | |
| | | CTCAGGGGTTTCTGATCGCTTCTCTGGCTCCAAG | |
| | | TCTGGCAACACGGCCTCCCTGACCATCTCTGGGC | |
| | | TCCAGGCTGAGGACGAGGCTGATTATTACTGCAG | |
| | | CTCATTTACAACCAGCAGCACTGTGGTTTTCGGC | |
| | | GGAGGGACCAAGCTGACCGTCCTAGGCCAGCCCA | |
| | | AGGCCGCCCCCTCCGTGACCCTGTTCCCCCCCTC | |
| | | CTCCGAGGAGCTGCAGGCCAACAAGGCCACCCTG | |
| | | GTGTGCCTGATCTCCGACTTCTACCCCGGCGCCG | |
| | | TGACCGTGGCCTGGAAGGCCGACTCCTCCCCCGT | |
| | | GAAGGCCGGCGTGGAGACCACCACCCCCTCCAAG | |
| | | CAGTCCAACAACAAGTACGCCGCCTCCTCCTACC | |
| | | TGTCCCTGACCCCCGAGCAGTGGAAGTCCCACCG | |
| | | GTCCTACTCCTGCCAGGTGACCCACGAGGGCTCC | |
| | | ACCGTGGAGAAGACCGTGGCCCCCACCGAGTGCT | |
| | | CCTGA | |

The antibodies of Table 1 include multispecific molecules, e.g., antibodies or antigen-binding fragments, that include the CDR-Hs and CDR-Ls, V_{H} and V_{L}, or HC and LC of those antibodies, respectively (including variants thereof as set forth herein).

In an embodiment, an antigen-binding domain that binds specifically to CoV-S, which may be included in a multispecific molecule, comprises:

(1)

(i) a heavy chain variable domain sequence that comprises CDR-H1, CDR-H2, and CDR-H3 amino acid sequences set forth in Table 1, and

(ii) a light chain variable domain sequence that comprises CDR-L1, CDR-L2, and CDR-L3 amino acid sequences set forth in Table 1;

or,

(2)

(i) a heavy chain variable domain sequence comprising an amino acid sequence set forth in Table 1, and

(ii) a light chain variable domain sequence comprising an amino acid sequence set forth in Table 1;

or,

(3)

(i) a heavy chain immunoglobulin sequence comprising an amino acid sequence set forth in Table 1, and

(ii) a light chain immunoglobulin sequence comprising an amino acid sequence set forth in Table 1.

In various embodiments, the present disclosure provides an isolated recombinant antibody or antigen-binding fragment thereof that specifically binds to a coronavirus spike protein (CoV-S), wherein the antibody has one or more of the following characteristics: (a) binds to CoV-S with an EC₅₀ of less than about 10⁻⁹M; (b) demonstrates an increase in survival in a coronavirus-infected animal after administration to said coronavirus-infected animal, as compared to a comparable coronavirus-infected animal without said administration; and/or (c) comprises three heavy chain complementarity determining regions (CDRs) (CDR-H1, CDR-H2, and CDR-H3) contained within a heavy chain variable region (HCVR) comprising an amino acid sequence having at least about 90% sequence identity to an HCVR of Table 1; and three light chain CDRs (CDR-L1, CDR-L2, and CDR-L3) contained within a light chain variable region (LCVR) comprising an amino acid sequence having at least about 90% sequence identity to an LCVR Table 1.

In various embodiments, a spike protein has at least 80% identity (e.g., at least 80%, 90%, 95%, 96%, 97%, 98%, 99%, or 100% identity) to the following sequence

(SEQ ID NO: 108):
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPRRARSVASQSIIAYTMSLGAENSVAYSNNSIAIPT
NFTISVTTEILPVSMTKTSVDCTMYICGDSTECSNLLLQYGSFCTQLNRALTGIAVEQDK
NTQEVFAQVKQIYKTPPIKDFGGFNFSQILPDPSKPSKRSFIEDLLFNKVTLADAGFIKQY
GDCLGDIAARDLICAQKFNGLTVLPPLLTDEMIAQYTSALLAGTITSGWTFGAGAALQIP
FAMQMAYRFNGIGVTQNVLYENQKLIANQFNSAIGKIQDSLSSTASALGKLQDVVNQN
AQALNTLVKQLSSNFGAISSVLNDILSRLDKVEAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN
ESLIDLQELGKYEQYIKWPWYIWLGFIAGLIAIVMVTIMLCCMTSCCSCLKGCCSCGSCC
KFDEDDSEPVLKGVKLHYT

In some embodiments, the present disclosure provides an isolated antibody or antigen-binding fragment thereof that binds a SARS-CoV-2 spike protein comprising the amino acid sequence set forth in SEQ ID NO: 108, wherein said isolated antibody or antigen-binding fragment comprises three heavy chain complementarity determining regions (CDRs) (HCDR1, HCDR2 and HCDR3) contained within a heavy chain variable region (HCVR) comprising the amino acid sequence set forth in SEQ ID NO: 29, and three light chain complementarity determining regions (CDRs) (LCDR1, LCDR2 and LCDR3) contained within a light chain variable region (LCVR) comprising the amino acid sequence set forth in SEQ ID NO: 33.

In some embodiments, the HCDR1 comprises the amino acid sequence set forth in SEQ ID NO: 30, the HCDR2 comprises the amino acid sequence set forth in SEQ ID NO: 31, the HCDR3 comprises the amino acid sequence set forth in SEQ ID NO: 32, the LCDR1 comprises the amino acid sequence set forth in SEQ ID NO: 34, the LCDR2 comprises the amino acid sequence set forth in SEQ ID NO: 35, and the LCDR3 comprises the amino acid sequence set forth in SEQ ID NO: 36. In some embodiments, the isolated antibody or antigen-binding fragment thereof comprises an HCVR that comprises the amino acid sequence set forth in SEQ ID NO: 29. In some embodiments, the isolated antibody or antigen-binding fragment thereof comprises an LCVR that comprises the amino acid sequence set forth in SEQ ID NO: 33. In some embodiments, the isolated antibody or antigen-binding fragment thereof comprises an HCVR that comprises the amino acid sequence set forth in SEQ ID NO: 29 and an LCVR that comprises the amino acid sequence set forth in SEQ ID NO: 33.

In some embodiments, the present disclosure provides an isolated antibody that binds a SARS-CoV-2 spike protein comprising the amino acid sequence set forth in SEQ ID NO: 108, wherein said isolated antibody comprises an immunoglobulin constant region, three heavy chain complementarity determining regions (CDRs) (HCDR1, HCDR2 and HCDR3) contained within a heavy chain variable region (HCVR) comprising the amino acid sequence set forth in SEQ ID NO: 29, and three light chain complementarity determining regions (CDRs) (LCDR1, LCDR2 and LCDR3) contained within a light chain variable region (LCVR) comprising the amino acid sequence set forth in SEQ ID NO: 33.

In some embodiments, the HCDR1 comprises the amino acid sequence set forth in SEQ ID NO: 30, the HCDR2 comprises the amino acid sequence set forth in SEQ ID NO: 31, the HCDR3 comprises the amino acid sequence set forth in SEQ ID NO: 32, the LCDR1 comprises the amino acid sequence set forth in SEQ ID NO: 34, the LCDR2 comprises the amino acid sequence set forth in SEQ ID NO: 35, and the LCDR3 comprises the amino acid sequence set forth in SEQ ID NO: 36. In some embodiments, the isolated antibody comprises an HCVR that comprises the amino acid sequence set forth in SEQ ID NO: 29 and an LCVR that comprises the amino acid sequence set forth in SEQ ID NO: 33. In some embodiments, the isolated antibody comprises a heavy chain comprising the amino acid sequence set forth in SEQ ID NO: 37 and a light chain comprising the amino acid sequence set forth in SEQ ID NO: 38. In some cases, the immunoglobulin constant region is an IgG1 constant region. In some cases, the isolated antibody is a recombinant antibody. In some cases, the isolated antibody is multispecific.

In some embodiments, the present disclosure provides a pharmaceutical composition comprising an isolated antibody as discussed above or herein, and a pharmaceutically acceptable carrier or diluent.

In some cases, an antibody or antigen-binding fragment thereof comprises three heavy chain CDRs (HCDR1, HCDR2 and HCDR3) contained within an HCVR comprising the amino acid sequence set forth in SEQ ID NO: 69, and three light chain CDRs (LCDR1, LCDR2 and LCDR3) contained within an LCVR comprising the amino acid sequence set forth in SEQ ID NO: 73. In some cases, an antibody or antigen-binding fragment thereof comprises: HCDR1, comprising the amino acid sequence set forth in SEQ ID NO: 70; HCDR2, comprising the amino acid sequence set forth in SEQ ID NO: 71; HCDR3, comprising the amino acid sequence set forth in SEQ ID NO: 72; LCDR1, comprising the amino acid sequence set forth in SEQ ID NO: 74; LCDR2, comprising the amino acid sequence set forth in SEQ ID NO: 75; and LCDR3, comprising the amino acid sequence set forth in SEQ ID NO: 76. In some cases, an antibody or antigen-binding fragment thereof comprises an HCVR comprising the amino acid sequence set forth in SEQ ID NO: 69 and an LCVR comprising the amino acid sequence set forth in SEQ ID NO: 73. In some cases, an antibody or antigen-binding fragment thereof comprises a heavy chain comprising the amino acid sequence set forth in SEQ ID NO: 77 and a light chain comprising the amino acid sequence set forth in SEQ ID NO: 78.

In some embodiments, the present disclosure provides an isolated antibody or antigen-binding fragment thereof that binds a SARS-CoV-2 spike protein comprising the amino acid sequence set forth in SEQ ID NO: 108, wherein said isolated antibody or antigen-binding fragment comprises three heavy chain complementarity determining regions (CDRs) (HCDR1, HCDR2 and HCDR3) contained within a heavy chain variable region (HCVR) comprising the amino acid sequence set forth in SEQ ID NO: 69, and three light chain complementarity determining regions (CDRs) (LCDR1, LCDR2 and LCDR3) contained within a light chain variable region (LCVR) comprising the amino acid sequence set forth in SEQ ID NO: 73.

In some embodiments, the HCDR1 comprises the amino acid sequence set forth in SEQ ID NO: 70, the HCDR2 comprises the amino acid sequence set forth in SEQ ID NO: 71, the HCDR3 comprises the amino acid sequence set forth in SEQ ID NO: 72, the LCDR1 comprises the amino acid sequence set forth in SEQ ID NO: 74, the LCDR2 comprises the amino acid sequence set forth in SEQ ID NO: 75, and the LCDR3 comprises the amino acid sequence set forth in SEQ ID NO: 76. In some embodiments, the isolated antibody or antigen-binding fragment thereof comprises an HCVR that comprises an amino acid sequence set forth in SEQ ID NO: 69. In some embodiments, the isolated antibody or antigen-binding fragment thereof comprises an LCVR that comprises an amino acid sequence set forth in SEQ ID NO: 73. In some embodiments, the isolated antibody or antigen-binding fragment thereof comprises an HCVR that comprises an amino acid sequence set forth in SEQ ID NO: 69 and an LCVR that comprises an amino acid sequence set forth in SEQ ID NO: 73.

In some embodiments, the present disclosure provides an isolated antibody that binds a SARS-CoV-2 spike protein comprising the amino acid sequence set forth in SEQ ID NO: 108, wherein said isolated antibody comprises an immunoglobulin constant region, three heavy chain complementarity determining regions (CDRs) (HCDR1, HCDR2 and HCDR3) contained within a heavy chain variable region (HCVR) comprising the amino acid sequence set forth in SEQ ID NO: 69, and three light chain complementarity determining regions (CDRs) (LCDR1, LCDR2 and LCDR3) contained within a light chain variable region (LCVR) comprising the amino acid sequence set forth in SEQ ID NO: 73.

In some embodiments, the HCDR1 comprises the amino acid sequence set forth in SEQ ID NO: 70, the HCDR2 comprises the amino acid sequence set forth in SEQ ID NO: 71, the HCDR3 comprises the amino acid sequence set forth in SEQ ID NO: 72, the LCDR1 comprises the amino acid sequence set forth in SEQ ID NO: 74, the LCDR2 comprises the amino acid sequence set forth in SEQ ID NO: 75, and the LCDR3 comprises the amino acid sequence set forth in SEQ ID NO: 76. In some embodiments, the isolated antibody comprises an HCVR that comprises the amino acid sequence set forth in SEQ ID NO: 69 and an LCVR that comprises the amino acid sequence set forth in SEQ ID NO: 73. In some embodiments, the isolated antibody comprises a heavy chain comprising the amino acid sequence set forth in SEQ ID NO: 77 and a light chain comprising the amino acid sequence set forth in SEQ ID NO: 78. In some cases, the immunoglobulin constant region is an IgG1 constant region. In some cases, the isolated antibody is a recombinant antibody. In some cases, the isolated antibody is multispecific.

In some embodiments, a pharmaceutical composition further comprises a second therapeutic agent. In some cases, the second therapeutic agent is selected from the group consisting of: a second antibody, or an antigen-binding fragment thereof, that binds a SARS-CoV-2 spike protein comprising the amino acid sequence set forth in SEQ ID NO: 108, an anti-inflammatory agent, an antimalarial agent, and an antibody or antigen-binding fragment thereof that binds TMPRSS2.

In certain embodiments in which the epitope of an antibody of interest is known, frequency of variations in the amino acids of the epitope can be used to determine the frequency of subjects that include an epitope bound or expected to be bound by the antibody of interest. For example, in a clinical context, genomes encoding the target antigen of an antibody can be isolated from subjects and analyzed for whether the isolated genomes encode an epitope of the antibody (e.g., an antigen sequence with which the antibody binds or is expected to bind) or a different sequence (e.g., a sequence that corresponds to the epitope but is not a sequence with which the antibody binds or is expected to bind). If a number of distinct epitopes are compared, antibodies targeting epitopes that are more conserved in a therapeutic population can generally be preferred to antibodies targeting epitopes that are less conserved in the therapeutic population.

Variation in an antigen, and particularly in an epitope, of a therapeutic antibody can be evaluated in subjects having received antibody therapy to evaluate putative escape variants. Therapeutic intervention, e.g., by antibody therapy, results in selective pressure for variants that are less susceptible to the intervention (escape variants). One example of escape variants is selection for a pathogen genome mutation that causes the pathogen to be less susceptible to treatment with an antibody therapy. For instance, a pathogen genome mutation can be a change in the epitope of a therapeutic antibody, such that the antibody no longer binds its target antigen. Methods and systems of the present disclosure can be used to evaluate putative escape variant selection in subjects having received an antibody therapy by isolating genomes encoding the target antigen of antibody from the subjects after treatment and analyzing the sequences for variation in the amino acid sequence of the antigen and/or epitope. Variations in the epitope as compared to a subject sequence (e.g., a reference sequence) that the antibody is able to bind can be identified as putative escape variants.

Analysis of variation in an antigen or epitope can also be used to determine whether subjects that have not received a particular antibody therapy are likely to respond to the antibody therapy. Subjects that include genomic sequences (e.g., pathogen genomic sequences) encoding an epitope sequence that matches a sequence bound or expected to be bound by the antibody therapy can be classified as subjects likely to respond to the antibody therapy. Conversely, subjects that have genomic sequences (e.g., pathogen genomic sequences) encoding amino acids corresponding to the epitope sequence that do not match a sequence bound or expected to be bound by the antibody therapy can be classified as subjects not likely to respond to the antibody therapy. Accordingly, methods and systems of the present disclosure can be used in personalized medicine applications in which subjects likely to respond to an antibody therapy are selected for treatment with that therapy and individuals not likely to respond to the antibody therapy are not selected for treatment with that therapy.

Exemplary Methods and Systems for Application

As will be appreciated from the present disclosure, methods and systems provided here can be useful in various applications at least in party by varying query sequences, subject sequences, and/or analysis of pairwise comparisons between query sequences and subject sequences.

In various embodiments, methods and systems of the present disclosure include steps of obtaining and/or selecting query and (if different from the query) subject sequences; extracting coding sequences from query and subject sequences; pairwise comparison of all query extracted coding sequences and all subject extracted coding sequences, producing data relating to one or more categorization factors (e.g., percent identity, percent coverage, coverage length, percent identity over a predetermined coverage length, E-value, number of mutations, percent mutation, and/or phylogeny (e.g., phylogenetic groupings and/or phylogenetic relationships)) for each comparison; categorizing compared sequences into one or more categorized sequence groups based on one or more threshold values for one or more categorization factors (e.g., where each categorized sequence group is assigned a similarity score); filtering one or more categorized sequence groups from further analysis (e.g., based on a similarity score threshold), translating coding sequences into amino acid sequences; aligning translated coding sequences; and determining conservation and/or variability for each of one or more subject sequences.

In various embodiments, methods and systems of the present disclosure include steps of obtaining and/or selecting query and (if different from the query) subject sequences; extracting coding sequences from query sequences; pairwise comparison of all query extracted coding sequences and all subject sequences, form which subject sequences coding sequences have not been extracted, producing data relating to one or more categorization factors (e.g., percent identity, percent coverage, coverage length, percent identity over a predetermined coverage length, E-value, number of mutations, percent mutation, and/or phylogeny (e.g., phylogenetic groupings and/or phylogenetic relationships)) for each comparison; categorizing compared sequences into one or more categorized sequence groups based on one or more threshold values for one or more categorization factors (e.g., where each categorized sequence group is assigned a similarity score); filtering one or more categorized sequence groups from further analysis (e.g., based on a similarity score threshold), translating coding sequences into amino acid sequences; aligning translated coding sequences; and determining conservation and/or variability for each of one or more subject sequences or portions thereof.

An exemplary schematic is provided in FIG. 48.

In various embodiments, methods and systems of the present disclosure include steps of obtaining and/or selecting query and (if different from the query) subject sequences; extracting coding sequences from query and subject sequences; translating coding sequences into amino acid sequences; pairwise comparison of all query translated coding sequences and all subject translated coding sequences, producing data relating to one or more categorization factors (e.g., percent identity, percent coverage, coverage length, percent identity over a predetermined coverage length, E-value, number of mutations, percent mutation, and/or phylogeny (e.g., phylogenetic groupings and/or phylogenetic relationships)) for each comparison; categorizing compared sequences into one or more categorized sequence groups based on one or more threshold values for one or more categorization factors (e.g., where each categorized sequence group is assigned a similarity score); filtering one or more categorized sequence groups from further analysis (e.g., based on a similarity score threshold); and determining conservation and/or variability for each subject sequence.

In various embodiments, extraction of coding sequences is based on annotation of a reference genomic sequence. Annotation of a reference genomic sequence can include identification, demarcation, or isolation of coding sequences. Annotated reference genomic sequences are available in publicly accessible databases and/or can be generated or modified by a user. Accordingly, in various embodiments in which a subject sequence is a reference genomic sequence, identification and/or extraction of query coding sequences can be based on available or user-defined annotation of coding sequences, e.g., in a reference genomic sequence. In various embodiments, coding sequences of subject and/or query genomic sequences can be identified and/or extracted by alignment of the subject and/or query genomic sequences to an annotated reference genomic sequence and/or coding sequences thereof.

In various embodiments, extraction of coding sequences from query and subject sequences is based on detection of contiguous in-frame codons encoding at least about 20, 30, 40, 50, 60, 70, 80, 90, 100, 125, 150, 175, 200, 250, or 300 or more amino acids.

In various embodiments, pairwise comparison of query and subject sequences is based on a BLAST algorithm. BLAST algorithms are known in the art, including BLASTN for nucleotide sequences and BLASTP, gapped BLAST, and PSI-BLAST for amino acid sequences. BLAST algorithms align sequences and produce various data for each alignment including without limitation data providing percent identity, number of mutations, percent mutation, coverage length, percent coverage, and E-value.

Compared sequences can be categorized according to categorization factors as set forth in Table 2. Table 2 assigns similarity scores to categorized sequence groups based on percent coverage and number of mutations. After formation of categorized sequence groups, categorized sequence groups having a similarity score less than a particular threshold (e.g., similarity score less than 1, less than 0.95, or less than 0.8) can be filtered out from further analysis.

Coding sequences (e.g., remaining categorized groups of coding sequences) can be translated into amino acid sequences by applying a relevant genetic code (e.g., the human genetic code). Translated coding sequences can be aligned. As noted above, alignment can be accomplished using a BLAST algorithm. Conservation and/or variability of sequences can then be determined. Various analyses set forth in methods and systems of the present disclosure do not require filtering or selection after alignment of amino acid sequences. Alignment absent further selection provides valuable information. For instance, in various embodiments, alignment of amino acid sequence provides information such as conservation at aligned positions (e.g., the percent of aligned sequences that include the same amino acid as a reference at each of one or more aligned positions) and sequence variation at aligned positions (e.g., the number and frequency of different amino acids that can occur at each aligned position). To the extent sequences are selected in certain embodiments following amino acid alignment, selection can be by a user, e.g., according to criteria applied to information produced by alignment of amino acid sequences. Thus, in various embodiments, no filters are applied to amino acid sequences, e.g., no threshold values are used for selection of amino acid sequences or portions thereof. In some embodiments, conserved or variable sequences can be selected based on a threshold as disclosed herein.

In various embodiments in which conservation and/or variability are evaluated, the query is a first collection of a sequences and the subject is a second different collection of sequences. In various embodiments, the query is a first collection of a sequences and the subject is the same collection of sequences. In various embodiments in which conservation and/or variability are evaluated, the query is a first collection of a sequences and the subject is a single sequences (e.g., a sequence of interest).

In certain embodiments, conservation and/or variability can be evaluated with respect to a pairwise comparison in which the query is a first collection of sequences from plurality of organisms of a particular species (e.g., a particular pathogen) and the subject is the same collection of sequences. Various such embodiments can produce data from pairwise comparisons that can be used to determine conserved sequences of the particular species and/or variable sequences of the particular species. Conserved sequences can be, e.g., selected or use an antigen or epitope in antibody or vaccine development. Conserved sequences can be traits under positive selection, e.g., evolutionary survival selection pressure and/or selection for antibiotic resistance, e.g., of a pathogen in human subjects. Variable sequences can be, e.g., selected as targets for laboratory engineering (e.g., genetic engineering), selected as targets for phylogenetic analysis, and/or identified as sequences undergoing evolutionary diversification. Variation in sequences can also be used to produce a listing or database of possible sequences (e.g., possible amino acid sequences) which can be used, for example, to generate possible masses for mass spectrometry analyses.

In certain embodiments, conservation and/or variability can be evaluated with respect to a pairwise comparison in which the query is a collection of sequences from a plurality of organisms of a particular species (e.g., a particular pathogen) and the subject includes one or more sequences from a particular strain or organism. In various embodiments, the query includes sequences from a plurality of organisms from different samples (e.g., a plurality of clinical isolates of a pathogen). In various embodiments, the subject is a laboratory strain. In certain embodiments, measured conservation and/or variability between subject sequences and query sequences can be used to determine how representative the subject strain or organism is of the query sequences. In various embodiments, a determination of whether a subject strain is representative of the query sequences is determined at the organismal level and/or by evaluation of all aligned sequences. In various embodiments, a determination at the organismal level can be based on a phylogentic analysis. For example, phylogetic analysis can identify one or more sequences of interest in clusters and determine sizes of all clusters.

Variation in sequences can also be used to produce a listing or database of possible sequences (e.g., possible amino acid sequences) which can be used, for example, to generate a listing or database of possible masses for mass spectrometry analyses.

To provide one particular example, methods and systems of the present disclosure can be used in various embodiments in which sequences of a virus such as SARS-CoV-2 are analyzed. In various embodiments, application of methods and systems of the present disclosure to analysis of SARS-CoV-2 sequences can include as the subject one or more reference SARS-CoV-2 sequences, such as the known SARS-CoV-2 reference genomic sequence publicly available as GenBank Accession No. MN908947. In some embodiments the subject can be or include a portion of a SARS-CoV-2 reference genomic sequence (e.g., a portion of GenBank accession: MN908947) that encodes an amino acid sequence, e.g., the SARS-CoV-2 spike protein or a portion thereof (e.g., the SARS-CoV-2 spike receptor-binding domain (RBD)). In various embodiments, the query sequence(s) can be a plurality of SARS-CoV-2 genomic sequences or coding sequences extracted therefrom. For example, at least about 120,000 SARS-CoV-2 genomic sequences are available through the global initiative on sharing all influenza data (GISAID) database (Hypertext Transfer Protocol www.gisaid.org/). Alternative or additional query sequences can be derived from infected subjects. Coding sequences can be extracted from SARS-CoV-2 genomic sequences, e.g., according to the general schematic found in FIG. 26. Pairwise comparison of all query extracted coding sequences and all subject extracted coding sequences can be performed as illustrated in the general schematic found in FIG. 27. Pairwise comparison of the query and subject SARS-CoV-2 sequences produces data relating to categorization factors including percent identity, percent coverage, coverage length, percent identity over a predetermined coverage length, E-value, number of mutations, percent mutation, and phylogeny (e.g., phylogenetic groupings and/or phylogenetic relationships for each comparison. These data allow various further analyses. Summary tables including resulting sequence comparison data can be prepared, e.g., as illustrated by the general layout found in the table of FIG. 28, showing a subset of categorization factors. Moreover, each comparison of a query SARS-CoV-2 sequence to a reference SARS-CoV-2 can be categorized into one or more categorized sequence groups based on one or more threshold values for one or more categorization factors. In some embodiments, one or more threshold values for one or more categorization factors can be integrated into a single metric, e.g., by assignment of a similarity score as illustrated in Table 2. In some embodiments, thresholds for one or more categorization factors (or for a similarity score determined based on two or more such thresholds) can be used to categorize SARS-CoV-2 sequence comparison results into categories, where one or more categories include query sequences that are more similar to reference sequence or portion thereof and one or more different categories include query sequences that are less similar to a reference sequence or portion thereof. Accordingly, in various embodiments, sequences that are more similar to a reference sequence can be retained for further analysis with respect to the reference sequence or portion thereof and sequences that are less similar to a reference sequence or portion thereof can be excluded from further analysis. When a sequence that is more similar to a reference sequence or portion thereof is found in a query genomic sequence, that reference sequence or portion thereof can be referred to as “present” in the query genomic sequence, as generally indicated, e.g., in FIG. 28. Measures of conservation and/or variability can be displayed in graphs, heatmaps, phylogenies, ranked lists, and other formats (for general exemplification, see, e.g., FIGS. 29-33). Remaining SARS-CoV-2 sequences for each reference sequence or portion thereof can be translated and aligned and measures of amino acid conservation and/or variability of aligned sequences can be determined.

In various embodiments, BLAST parameters for comparison of nucleic acid sequences can be performed using BLAST default values or with any of the values provided in Table 4. In various embodiments, BLAST parameters for comparison of amino acid sequences can be performed using BLAST default values or with any of the values provided in Table 5. No particular set of values for any parameter or combination of parameters is required for use of systems and methods of the present disclosure.

TABLE 4
Nucleic acid comparison BLASTn parameters

| | Exemplary | Exemplary | Exemplary |
|---|---|---|---|
| Parameter | Range | Values | Default(s) |
| Cost to Open a | 0 to 10 | 0, 1, 2, 3, | 1 |
| Gap (“Gap Cost: | | 4, 5, 6 | |
| Existence”) | | | |
| Cost to Extend | 0 to 10 | 0, 1, 2, 3, | 1 |
| a Gap (“Gap Cost: | | 4, 5, 6 | |
| Extension”) | | | |
| Length of | 5 to 256 | 7, 11, 15, | 28 |
| Sequence of | | 16, 20, 24, | |
| Perfect Match | | 28, 32, 48, | |
| (“word size”) | | 64, 128, 256 | |
| Reward for Match | 1 to 15 | 1, 2, 3, 4 | 1 |
| (“Match Score”) | | | |
| Reward for | −1 to −15 | −1, −2, −3, | −2 |
| Mismatch | | −4, −5 | |
| (“Mismatch | | | |
| Score”) | | | |
| E-value (“Expect | 0 to 0.1 | 1e−50, 1e−40, | 0.05 |
| Threshold”) | | 1e−30, 1e−20, | |
| | | 1e−10, 1e−9, | |
| | | 1e−8, 1e−7, | |
| | | 1e−6, 1e−5, | |
| | | 1e−4, 1e−3, | |
| | | or 1e−2, 1e−1 | |

TABLE 5
Amino acid comparison BLASTp parameters

| | Exemplary | Exemplary | Exemplary |
|---|---|---|---|
| Parameter | Range | Values | Default(s) |
| Cost to Open a | 0 to 50 | 6, 7, 8, 9, | 11 |
| Gap (“Gap Cost: | | 10, 11, 12, | |
| Existence”) | | 13, 14, 15 | |
| Cost to Extend | 0 to 10 | 0, 1, 2, 3 | 1 |
| a Gap (“Gap Cost: | | | |
| Extension”) | | | |
| Length of Sequence | 2 to 20 | 2, 3, 6 | 6 |
| of Perfect Match | | | |
| (“word size”) | | | |
| E-value (“Expect | 0 to 0.2 | 1e−50, 1e−40, | 0.05 |
| Threshold”) | | 1e−30, 1e−20, | |
| | | 1e−10, 1e−9, | |
| | | 1e−8, 1e−7, | |
| | | 1e−6, 1e−5, | |
| | | 1e−4, 1e−3, | |
| | | or 1e−2, 1e−1 | |

| | |
|---|---|
| Reward for Match | Scoring matrix for match and mismatch rewards: |
| (“Match Score”) | Point Accepted Mutation (PAM) Matrix (e.g., PAM30, |
| Reward for Mismatch | PAM70, or PAM250); |
| (“Mismatch Score”) | Blocks Substitution Matrix (BLOSUM) (e.g. BLOSUM45, |
| | BLOSUM50, BLOSUM62, BLOSUM80, or BLOSUM90) |

### Exemplary Embodiments

The present disclosure includes, among other things, the following exemplary embodiments:

1. A method for identifying amino acid sequences as candidate antigens in the development of a therapy against a pathogen, comprising:

obtaining a plurality of complete or partial genomic sequences of different strains of the pathogen from a data structure;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

classifying each of a plurality of portions of the aligned amino acid sequences according to a level of conservation of said portion among the different strains of the pathogen;

selecting portions of the amino acid sequences classified as conserved, comparing the selected conserved sequences to human protein sequences, and further classifying the selected conserved sequences as identical or not identical to a human protein sequence; and

categorizing a selected conserved sequence not identical to a human protein sequence as a candidate antigen in the development of a therapy against the pathogen.

2. The method according to embodiment 1, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
3. The method according to embodiment 1 or embodiment 2, wherein the categorizing step comprises quantifying the measure of identity and the measure of coverage for each of a plurality of pairs, each of said pairs comprising an extracted coding sequence and a reference sequence.
4. The method according to any one of embodiments 1 to 3, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
5. The method according to embodiment 4, wherein the computing step comprises creating a matrix of said measures of similarity and rendering a graphical representation of said matrix, thereby displaying levels of conservation between the query sequences and subject sequences.
6. The method according to embodiment 5, wherein the graphical representation comprises one or more of a heatmap, a graph, and a phylogeny.
7. The method according to any one of embodiments 1 to 6, wherein the measure of identity comprises number of mutations.
8. The method according to any one of embodiments 1 to 7, wherein the measure of coverage comprises percent coverage.
9. The method according to any one of embodiments 1 to 8, wherein the measure of identity comprises calculating E-value.
10. The method according to any one of embodiments 1 to 9, wherein categorizing the selected conserved sequence as a candidate antigen further comprises determining the presence or absence of one or more amino acid domains in the selected conserved sequence.
11. The method according to any one of embodiments 1 to 10, wherein categorizing the selected conserved sequence as a candidate antigen further comprises determining whether the candidate antigen corresponds to a protein that is secreted or is exposed within a membrane and/or cell wall of the pathogen.
12. The method according to any one of embodiments 1 to 11, wherein categorizing the selected conserved sequence as a candidate antigen further comprises determining the presence of a transmembrane domain in a selected conserved sequence.
13. The method according to any one of embodiments 1 to 12, wherein the therapy comprises a vaccine and the method further comprises non-clinically evaluating the candidate antigen for immunogenicity.
14. The method according to embodiment 13, wherein the evaluating step comprises administering a polypeptide comprising the candidate antigen to an animal.
15. The method according to any one of embodiments 1 to 14, wherein the therapy comprises an antibody therapy, and the method further comprises producing an antibody or fragment thereof that specifically binds to an epitope on the candidate antigen.
16. The method according to any one of embodiments 1 to 15, wherein the pathogen is a virus.
17. The method according to embodiment 16, wherein the virus is methicillin-resistant Staphylococcus aureus (MRSA), Hepatitis B Virus (HBV), influenza, or Ebola virus.
18. The method according to embodiment 16, wherein the virus is a coronavirus.
19. The method according to embodiment 18, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
20. The method according to any one of embodiments 1 to 15, wherein the pathogen is a bacterium.
21. The method according to embodiment 20, wherein the bacterium is a Staphylococcus species or a Pseudomonas species.
22. A method of identifying one or more putative escape mutations after administration of a therapeutic agent to one or more subjects for treatment of a pathogen infection, comprising:

obtaining a plurality of complete or partial pathogen genomic sequences isolated from one or more subjects after administration of the therapeutic agent to each subject;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

identifying, in the aligned amino acid sequences, one or more amino acid variants more frequent in the aligned amino acid sequences than in a reference, said one or more amino acid variants being one or more putative escape mutations.

23. The method according to embodiment 22, wherein the reference comprises one or more complete or partial pathogen genomic sequences representative of a canonical pathogen sequence, one or more clinical strains of the pathogen, one or more earlier samples of pathogen from one or more of the subjects administered the therapeutic agent, or one or more samples of pathogen from subjects not administered the therapeutic agent.
24. The method according to embodiment 22 or embodiment 23, further comprising a step of determining whether one or more of the putative escape mutations decreases binding affinity of the therapeutic agent with a reference polypeptide.
25. The method according to any one of embodiments 22 to 24, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
26. The method according to any one of embodiments 22 to 25, wherein the categorizing step comprises quantifying the measure of identity and the measure of coverage for each of a plurality of pairs, each of said pairs comprising an extracted coding sequence and a reference sequence.
27. The method according to any one of embodiments 22 to 26, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
28. The method according to embodiment 27, wherein the computing step comprises creating a matrix of said measures of similarity and rendering a graphical representation of said matrix, thereby displaying levels of conservation between the query sequences and subject sequences.
29. The method according to embodiment 28, wherein the graphical representation comprises one or more of a heatmap, a graph, and a phylogeny.
30. The method according to any one of embodiments 22 to 29, wherein the measure of identity comprises number of mutations.
31. The method according to any one of embodiments 22 to 30, wherein the measure of coverage comprises percent coverage.
32. The method according to any one of embodiments 22 to 31, wherein the measure of identity comprises calculating E-value.
33. The method according to any one of embodiments 22 to 32, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

34. The method of any one of embodiments 22 to 33, wherein each portion of an amino acid sequence comprises one or more amino acid positions.
35. The method according to any one of embodiments 22 to 34, wherein the pathogen is a virus.
36. The method according to embodiment 35, wherein the virus is Methicillin-resistant Staphylococcus aureus (MRSA), Hepatitis B Virus (HBV), influenza, or Ebola virus.
37. The method according to embodiment 35, wherein the virus is a coronavirus.
38. The method according to embodiment 37, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
39. The method according to embodiment 38, wherein the coronavirus is SARS-CoV-2.
40. The method according to any one of embodiments 22 to 39, comprising evaluating a coronavirus spike (S) protein [e.g., MERS, SARS-CoV, or SARS-CoV2 spike (S) protein] or a receptor-binding domain (RBD) thereof.
41. The method according to any one of embodiments 22 to 40, wherein the therapeutic agent comprises an antibody.
42. The method according to embodiment 41, wherein the antibody binds SARS-CoV-2.
43. The method according to embodiment 42, wherein the antibody binds SARS-CoV-2 spike protein.
44. The method according to any one of embodiments 41 to 43, wherein the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3.
45. The method according to any one of embodiments 22 to 34, wherein the pathogen is a bacterium.
46. The method according to embodiment 45, wherein the bacterium is a Staphylococcus species or a Pseudomonas species.
47. A method of administering a therapeutic agent for treatment of a pathogen infection to a subject in need thereof, comprising:

selecting a conserved portion of an amino acid sequence by:
obtaining a plurality of complete or partial genomic sequences of different strains of the pathogen from a data structure;
extracting, by a processor of a computing device, coding sequences from the genomic sequences;
categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
selecting coding sequences from among the categorized coding sequences according to the measure of identity and the measure of coverage;
converting, by the processor, the selected coding sequences into corresponding amino acid sequences;
aligning, by the processor, the amino acid sequences;
classifying each of a plurality of portions of the aligned amino acid sequences according to a level of conservation of said portion among the different strains of the pathogen; and

selecting a conserved portion of the aligned amino acid sequences; and administering the therapeutic agent to a subject if a complete or partial pathogen genomic sequence isolated from the subject encodes the conserved portion of an amino acid sequence, wherein the therapeutic agent selectively binds the conserved portion of the amino acid sequence.

48. The method according to embodiment 47, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
49. The method according to embodiment 47 or embodiment 48, wherein the categorizing step comprises quantifying the measure of identity and the measure of coverage for each of a plurality of pairs, each of said pairs comprising an extracted coding sequence and a reference sequence.
50. The method according to any one of embodiments 47 to 49, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
51. The method according to embodiment 50, wherein the computing step comprises creating a matrix of said measures of similarity and rendering a graphical representation of said matrix, thereby displaying levels of conservation between the query sequences and subject sequences.
52. The method according to embodiment 51, wherein the graphical representation comprises one or more of a heatmap, a graph, and a phylogeny.
53. The method according to any one of embodiments 47 to 52, wherein the measure of identity comprises number of mutations.
54. The method according to any one of embodiments 47 to 53, wherein the measure of coverage comprises percent coverage.
55. The method according to any one of embodiments 47 to 54, wherein the measure of identity comprises calculating E-value.
56. The method according to any one of embodiments 47 to 55, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

57. The method of any one of embodiments 47 to 56, wherein each portion of an amino acid sequence comprises one or more amino acid positions.
58. The method according to any one of embodiments 47 to 57, wherein the pathogen is a virus.
59. The method according to embodiment 58, wherein the virus is Methicillin-resistant Staphylococcus aureus (MRSA), Hepatitis B Virus (HBV), influenza, or Ebola virus.
60. The method according to embodiment 58, wherein the virus is a coronavirus.
61. The method according to embodiment 60, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
62. The method according to embodiment 61, wherein the coronavirus is SARS-CoV-2.
63. The method according to any one of embodiments 47 to 62, comprising evaluating a coronavirus spike (S) protein [e.g., MERS, SARS-CoV, or SARS-CoV2 spike (S) protein] or a receptor-binding domain (RBD) thereof.
64. The method according to any one of embodiments 47 to 63, wherein the therapeutic agent comprises an antibody.
65. The method according to embodiment 64, wherein the antibody binds SARS-CoV-2.
66. The method according to embodiment 65, wherein the antibody binds SARS-CoV-2 spike protein.
67. The method according to any one of embodiments 64 to 66, wherein the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3.
68. The method according to any one of embodiments 47 to 57, wherein the pathogen is a bacterium.
69. The method according to embodiment 68, wherein the bacterium is a Staphylococcus species or a Pseudomonas species.
70. A method for selecting a therapeutic agent for treatment of subjects infected with a pathogen, comprising:

obtaining a plurality of complete or partial genomic sequences of different strains of the pathogen from a data structure;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

classifying each of a plurality of portions of the aligned amino acid sequences according to a level of conservation of said portion among the different strains of the pathogen, thereby identifying a conserved portion of a coding sequence representative of the pathogen; and

selecting a therapeutic agent that binds the conserved coding sequence as a treatment for subjects infected with the pathogen.

71. The method according to embodiment 70, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
72. The method according to embodiment 70 or embodiment 71, wherein the categorizing step comprises quantifying the measure of identity and the measure of coverage for each of a plurality of pairs, each of said pairs comprising an extracted coding sequence and a reference sequence.
73. The method according to any one of embodiments 70 to 72, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
74. The method according to embodiment 73, wherein the computing step comprises creating a matrix of said measures of similarity and rendering a graphical representation of said matrix, thereby displaying levels of conservation between the query sequences and subject sequences.
75. The method according to embodiment 74, wherein the graphical representation comprises one or more of a heatmap, a graph, and a phylogeny.
76. The method according to any one of embodiments 70 to 75, wherein the measure of identity comprises number of mutations.
77. The method according to any one of embodiments 70 to 76, wherein the measure of coverage comprises percent coverage.
78. The method according to any one of embodiments 70 to 77, wherein the measure of identity comprises calculating E-value.
79. The method according to any one of embodiments 70 to 78, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

80. The method of any one of embodiments 70 to 79, wherein each portion of an amino acid sequence comprises one or more amino acid positions.
81. The method according to embodiment 80, wherein the method further comprises non-clinically evaluating the therapeutic agent as a vaccine or component thereof.
82. The method according to embodiment 81, wherein the evaluating step comprises administering the therapeutic agent to an animal.
83. The method according to any one of embodiments 70 to 82, wherein the pathogen is a virus.
84. The method according to embodiment 83, wherein the virus is Methicillin-resistant Staphylococcus aureus (MRSA), Hepatitis B Virus (HBV), influenza, or Ebola virus.
85. The method according to embodiment 83, wherein the virus is a coronavirus.
86. The method according to embodiment 85, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
87. The method according to embodiment 86, wherein the coronavirus is SARS-CoV-2.
88. The method according to any one of embodiments 70 to 87, comprising evaluating a coronavirus spike (S) protein [e.g., MERS, SARS-CoV, or SARS-CoV2 spike (S) protein] or a receptor-binding domain (RBD) thereof.
89. The method according to any one of embodiments 70 to 88, wherein the therapeutic agent comprises an antibody.
90. The method according to embodiment 89, wherein the antibody binds SARS-CoV-2.
91. The method according to embodiment 90, wherein the antibody binds SARS-CoV-2 spike protein.
92. The method according to any one of embodiments 89 to 91, wherein the antibody comprises at least one antibody, heavy chain (HC), light chain (LC), heavy chain variable region (HCVR), light chain variable region (LCVR), heavy chain complementarity determining region (HCDR), or light chain CDR (LCDR) according to Table 3.
93. The method according to any one of embodiments 70 to 82, wherein the pathogen is a bacterium.
94. The method according to embodiment 93, wherein the bacterium is a Staphylococcus species or a Pseudomonas species.
95. A method for assessing conservation of portions of amino acid sequences representative of a pathogen, comprising:

obtaining a plurality of complete or partial genomic sequences of different strains of the pathogen from a data structure;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences; and

identifying a level of conservation of one or more portions of amino acid sequences representative of the pathogen using the aligned amino acid sequences.

96. The method according to embodiment 95, wherein one or more of the portions is identified as a candidate antigen in the development of a therapy against the pathogen.
97. The method according embodiment 95 or embodiment 96, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
98. The method according to any one of embodiments 95 to 97, wherein the categorizing step comprises quantifying the measure of identity and the measure of coverage for each of a plurality of pairs, each of said pairs comprising an extracted coding sequence and a reference sequence.
99. The method according to any one of embodiments 95 to 98, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
100. The method according to embodiment 99, wherein the computing step comprises creating a matrix of said measures of similarity and rendering a graphical representation of said matrix, thereby displaying levels of conservation between the query sequences and subject sequences.
101. The method according to embodiment 100, wherein the graphical representation comprises one or more of a heatmap, a graph, and a phylogeny.
102. The method according to any one of embodiments 95 to 101, wherein the measure of identity comprises number of mutations.
103. The method according to any one of embodiments 95 to 102, wherein the measure of coverage comprises percent coverage.
104. The method according to any one of embodiments 95 to 103, wherein the measure of identity comprises calculating E-value.
105. The method according to any one of embodiments 95 to 104, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

106. The method of any one of embodiments 95 to 105, wherein each portion of an amino acid sequence comprises one or more amino acid positions.
107. The method according to any one of embodiments 95 to 106, wherein the pathogen is a virus.
108. The method according to embodiment 107, wherein the virus is Methicillin-resistant Staphylococcus aureus (MRSA), Hepatitis B Virus (HBV), influenza, or Ebola virus.
109. The method according to embodiment 107, wherein the virus is a coronavirus.
110. The method according to embodiment 109, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
111. The method according to embodiment 110, wherein the coronavirus is SARS-CoV-2.
112. The method of any one of embodiments 95 to 111, wherein the genomic sequences are SARS-CoV-2 genomic sequences and the reference sequence is a SARS-CoV-2 reference sequence.
113. The method according to any one of embodiments 95 to 112, comprising evaluating a coronavirus spike (S) protein [e.g., MERS, SARS-CoV, or SARS-CoV2 spike (S) protein] or a receptor-binding domain (RBD) thereof.
114. The method according to any one of embodiments 95 to 106, wherein the pathogen is a bacterium.
115. The method according to embodiment 114, wherein the bacterium is a Staphylococcus species or a Pseudomonas species.
116. A method for identifying whether an isolated pathogen is representative of a circulating strain, comprising:

obtaining a plurality of complete or partial genomic sequences of the circulating strain of the pathogen from a data structure;

identifying one or more conserved portions of said sequences of the circulating strain;

obtaining a plurality of complete or partial genomic sequences of the isolated pathogen; and

identifying whether said isolated pathogen is representative of the circulating strain by comparing at least a portion of said sequences of the isolated pathogen against the identified one or more conserved portions of the sequences of the circulating strain.

117. The method according to embodiment 116, wherein identifying one or more conserved portions of said sequences of the circulating strain comprises:

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences; and

classifying each of a plurality of portions of the aligned amino acid sequences according to a level of conservation of said portion among the aligned amino acid sequences.

118. The method according to embodiment 116 or embodiment 117, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
119. The method according to any one of embodiments 116 to 118, wherein the categorizing step comprises quantifying the measure of identity and the measure of coverage for each of a plurality of pairs, each of said pairs comprising an extracted coding sequence and a reference sequence.
120. The method according to any one of embodiments 116 to 119, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
121. The method according to embodiment 120, wherein the computing step comprises creating a matrix of said measures of similarity and rendering a graphical representation of said matrix, thereby displaying levels of conservation between the query sequences and subject sequences.
122. The method according to embodiment 121, wherein the graphical representation comprises one or more of a heatmap, a graph, and a phylogeny.
123. The method according to any one of embodiments 116 to 122, wherein the measure of identity comprises number of mutations.
124. The method according to any one of embodiments 116 to 123, wherein the measure of coverage comprises percent coverage.
125. The method according to any one of embodiments 116 to 124, wherein the measure of identity comprises calculating E-value.
126. The method according to any one of embodiments 116 to 125, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

127. The method of any one of embodiments 116 to 126, wherein each portion of an amino acid sequence comprises one or more amino acid positions.
128. The method according to any one of embodiments 116 to 127, wherein the pathogen is a virus.
129. The method according to embodiment 128, wherein the virus is Methicillin-resistant Staphylococcus aureus (MRSA), Hepatitis B Virus (HBV), influenza, or Ebola virus.
130. The method according to embodiment 128, wherein the virus is a coronavirus.
131. The method according to embodiment 130, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
132. The method according to embodiment 131, wherein the coronavirus is SARS-CoV-2.
133. The method according to any one of embodiments 116 to 132, comprising evaluating a coronavirus spike (S) protein [e.g., MERS, SARS-CoV, or SARS-CoV2 spike (S) protein] or a receptor-binding domain (RBD) thereof.
134. The method according to any one of embodiments 116 to 127, wherein the pathogen is a bacterium.
135. The method according to embodiment 134, wherein the bacterium is a Staphylococcus species or a Pseudomonas species.
136. A method for identifying a mass-to-charge ratio of a peptide representative of a pathogen, comprising:

obtaining a plurality of complete or partial genomic sequences of different strains of the pathogen from a data structure;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences; and

determining the mass-to-charge ratio of one or more of the amino acid sequences or portions thereof.

137. The method according to embodiment 136, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.
138. The method according to embodiment 136 or embodiment 137, wherein the categorizing step comprises quantifying the measure of identity and the measure of coverage for each of a plurality of pairs, each of said pairs comprising an extracted coding sequence and a reference sequence.
139. The method according to any one of embodiments 136 to 138, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
140. The method according to embodiment 139, wherein the computing step comprises creating a matrix of said measures of similarity and rendering a graphical representation of said matrix, thereby displaying levels of conservation between the query sequences and subject sequences.
141. The method according to embodiment 140, wherein the graphical representation comprises one or more of a heatmap, a graph, and a phylogeny.
142. The method according to any one of embodiments 136 to 141, wherein the measure of identity comprises number of mutations.
143. The method according to any one of embodiments 136 to 142, wherein the measure of coverage comprises percent coverage.
144. The method according to any one of embodiments 136 to 143, wherein the measure of identity comprises calculating E-value.
145. The method according to any one of embodiments 136 to 144, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

146. The method of any one of embodiments 136 to 145, wherein each portion of an amino acid sequence comprises one or more amino acid positions.
147. The method according to any one of embodiments 136 to 146, wherein the pathogen is a virus.
148. The method according to embodiment 147, wherein the virus is Methicillin-resistant Staphylococcus aureus (MRSA), Hepatitis B Virus (HBV), influenza, or Ebola virus.
149. The method according to embodiment 147, wherein the virus is a coronavirus.
150. The method according to embodiment 149, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
151. The method according to embodiment 150, wherein the coronavirus is SARS-CoV-2.
152. The method according to any one of embodiments 136 to 151, comprising evaluating a coronavirus spike (S) protein [e.g., MERS, SARS-CoV, or SARS-CoV2 spike (S) protein] or a receptor-binding domain (RBD) thereof.
153. The method according to any one of embodiments 136 to 146, wherein the pathogen is a bacterium.
154. The method according to embodiment 153, wherein the bacterium is a Staphylococcus species or a Pseudomonas species.
155. A method for identifying an amino acid sequence as a candidate antibiotic resistance marker, comprising:

obtaining a plurality of complete or partial plasmid sequences of a pathogenic bacterium from a data structure;

extracting, by a processor of a computing device, coding sequences from the plasmid sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

classifying each of a plurality of portions of the aligned amino acid sequences according to a level of conservation of said portion among the plurality of plasmid sequences;

selecting portions of the amino acid sequences classified as conserved; and

categorizing a selected conserved sequence as a candidate antibiotic resistance marker.

156. The method according to embodiment 155, further comprising identifying the candidate antibiotic resistance marker as a candidate according to one or more additional criteria comprising a presence of a transmembrane domain in a selected sequence.
157. The method according to embodiment 155 or embodiment 156, wherein the data structure comprises contigs, and wherein obtaining the plurality of complete or partial plasmid sequences from the data structure comprises merging, by the processor, overlapping contigs to produce at least some of the complete or partial plasmid sequences.
158. The method according to any one of embodiments 155 to 157, wherein the categorizing step comprises quantifying the measure of identity and the measure of coverage for each of a plurality of pairs, each of said pairs comprising an extracted coding sequence and a reference sequence.
159. The method according to any one of embodiments 155 to 158, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
160. The method according to embodiment 159, wherein the computing step comprises creating a matrix of said measures of similarity and rendering a graphical representation of said matrix, thereby displaying levels of conservation between the query sequences and subject sequences.
161. The method according to embodiment 160, wherein the graphical representation comprises one or more of a heatmap, a graph, and a phylogeny.
162. The method according to any one of embodiments 155 to 161, wherein the measure of identity comprises number of mutations.
163. The method according to any one of embodiments 155 to 162, wherein the measure of coverage comprises percent coverage.
164. The method according to any one of embodiments 155 to 163, wherein the measure of identity comprises calculating E-value.
165. The method according to any one of embodiments 155 to 164, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

166. The method of any one of embodiments 155 to 165, wherein each portion of an amino acid sequence comprises one or more amino acid positions.
167. The method according to any one of embodiments 155 to 166, wherein the bacterium is a Staphylococcus species or a Pseudomonas species.
168. A method for identifying one or more conserved portions of coding sequences representative of a plasmid, comprising:

obtaining a plurality of complete or partial plasmid sequences of a pathogenic bacterium from a data structure;

extracting, by a processor of a computing device, coding sequences from the plasmid sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences; and

classifying each of a plurality of portions of the amino acid sequences according to a level of conservation of said portion among the plurality of plasmid sequences, thereby identifying one or more conserved portions of coding sequences representative of the plasmid.

169. The method according to embodiment 168, wherein the data structure comprises contigs, and wherein obtaining the plurality of complete or partial plasmid sequences from the data structure comprises merging, by the processor, overlapping contigs to produce at least some of the complete or partial plasmid sequences.
170. The method according to embodiment 168 or embodiment 169, wherein the categorizing step comprises quantifying the measure of identity and the measure of coverage for each of a plurality of pairs, each of said pairs comprising an extracted coding sequence and a reference sequence.
171. The method according to any one of embodiments 168 to 170, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
172. The method according to embodiment 171, wherein the computing step comprises creating a matrix of said measures of similarity and rendering a graphical representation of said matrix, thereby displaying levels of conservation between the query sequences and subject sequences.
173. The method according to embodiment 172, wherein the graphical representation comprises one or more of a heatmap, a graph, and a phylogeny.
174. The method according to any one of embodiments 168 to 173, wherein the measure of identity comprises number of mutations.
175. The method according to any one of embodiments 168 to 174, wherein the measure of coverage comprises percent coverage.
176. The method according to any one of embodiments 168 to 175, wherein the measure of identity comprises calculating E-value.
177. The method according to any one of embodiments 168 to 176, comprising evaluating one or more of:

coding sequences of a nucleic acid that encodes a protein associated with the pathogen;

conserved sequences of a nucleic acid sequence that encodes a protein associated with the pathogen;

non-conserved sequences of a nucleic acid that encodes a protein;

conserved domains within a particular protein associated with the pathogen; and

non-conserved domains within a particular protein associated with the pathogen.

178. The method of any one of embodiments 168 to 177, wherein each portion of an amino acid sequence comprises one or more amino acid positions.
179. The method according to any one of embodiments 168 to 178, wherein the bacterium is a Staphylococcus species or a Pseudomonas species.
180. A system for automatically identifying one or more conserved portions of coding sequences representative of a pathogen, the system comprising:

a processor; and

a memory having instructions thereon, the instructions, when executed by the processor, causing the processor to:
obtain a plurality of complete or partial genomic sequences of different strains of the pathogen from a data structure;
extract, by the processor, coding sequences from the genomic sequences;
categorize, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
select coding sequences from among the categorized coding sequences according to the measure of identity and the measure of coverage;
convert, by the processor, the selected coding sequences into corresponding amino acid sequences;
align, by the processor, the amino acid sequences; and
classify each of a plurality of portions of the aligned amino acid sequences according to a level of conservation of said portion among the different strains of the pathogen, thereby identifying one or more conserved portions of coding sequences representative of the pathogen.
   181. The system according to embodiment 180, wherein the instructions, when executed by the processor, cause the processor to compute, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
   182. The system according to embodiment 181, wherein the instructions, when executed by the processor, cause the processor to create a matrix of said measures of similarity and render a graphical representation of said matrix, thereby displaying levels of conservation between the query sequences and subject sequences.
   183. The system according to embodiment 182, wherein the graphical representation comprises one or more of a heatmap, a graph, and a phylogeny.
   184. The system according to any one of embodiments 180 to 183, wherein the data structure comprises contigs, and wherein the instructions, when executed by the processor, cause the processor to obtain the plurality of complete or partial genomic sequences of different strains of the pathogen by merging, by the processor, overlapping contigs to produce at least some of the complete or partial genomic sequences.
   185. The system according to any one of embodiments 180 to 184, wherein the instructions, when executed by the processor, cause the processor to evaluate one or more of:

coding sequences of a nucleic acid that encodes a protein associated with the pathogen;

conserved sequences of a nucleic acid sequence that encodes a protein associated with the pathogen

non-conserved sequences of a nucleic acid that encodes a protein;

conserved domains within a particular protein associated with the pathogen; and

non-conserved domains within a particular protein associated with the pathogen.

186. The system according to any one of embodiments 180 to 185, wherein the instructions, when executed by the processor, cause the processor to evaluate a coronavirus spike (S) protein [e.g., MERS, SARS-CoV, or SARS-CoV2 spike (S) protein] or a receptor-binding domain (RBD) thereof.
187. The system according to any one of embodiments 180 to 186, wherein the pathogen is a virus.
188. The system according to embodiment 187, wherein the virus is Methicillin-resistant Staphylococcus aureus (MRSA), Hepatitis B Virus (HBV), influenza, or Ebola virus.
189. The system according to embodiment 187, wherein the virus is a coronavirus.
190. The system according to embodiment 189, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
191. The system according to embodiment 190, wherein the coronavirus is SARS-CoV-2.
192. The system according to any one of embodiments 180 to 186, wherein the pathogen is a bacterium.
193. The system according to embodiment 192, wherein the bacterium is a Staphylococcus species or a Pseudomonas species.
194. A system for automatically identifying one or more conserved portions of coding sequences representative of a plasmid, the system comprising:

a processor; and

a memory having instructions thereon, the instructions, when executed by the processor, causing the processor to:
obtain a plurality of complete or partial plasmid sequences of a pathogenic bacterium from a data structure;
extract, by the processor, coding sequences from the plasmid sequences;
categorize, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
select coding sequences from among the categorized coding sequences according to the measure of identity and the measure of coverage;
convert, by the processor, the selected coding sequences into corresponding amino acid sequences;
align, by the processor, the amino acid sequences; and
classify each of a plurality of portions of the amino acid sequences according to a level of conservation of said portion among the plurality of plasmid sequences, thereby identifying one or more conserved portions of coding sequences representative of the plasmid.
   195. The system according to embodiment 194, wherein the instructions, when executed by the processor, cause the processor to compute, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.
   196. The system according to embodiment 195, wherein the instructions, when executed by the processor, cause the processor to create a matrix of said measures of similarity and render a graphical representation of said matrix, thereby displaying levels of conservation between the query sequences and subject sequences.
   197. The system according to embodiment 196, wherein the graphical representation comprises one or more of a heatmap, a graph, and a phylogeny.
   198. The system according to any one of embodiments 194 to 197, wherein the data structure comprises contigs, and wherein the instructions, when executed by the processor, cause the processor to obtain the plurality of complete or partial plasmid sequences of a pathogenic bacterium by merging, by the processor, overlapping contigs to produce at least some of the complete or partial plasmid sequences.
   199. The system according to any one of embodiments 194 to 198, wherein the instructions, when executed by the processor, cause the processor to evaluate one or more of:

coding sequences of a nucleic acid that encodes a protein associated with the pathogen;

conserved sequences of a nucleic acid sequence that encodes a protein associated with the pathogen

non-conserved sequences of a nucleic acid that encodes a protein;

conserved domains within a particular protein associated with the pathogen; and

non-conserved domains within a particular protein associated with the pathogen.

200. The system according to any one of embodiments 194 to 199, wherein the instructions, when executed by the processor, cause the processor to evaluate a coronavirus spike (S) protein [e.g., MERS, SARS-CoV, or SARS-CoV2 spike (S) protein] or a receptor-binding domain (RBD) thereof.
201. The system according to any one of embodiments 194 to 200, wherein the pathogen is a virus.
202. The system according to embodiment 201, wherein the virus is Methicillin-resistant Staphylococcus aureus (MRSA), Hepatitis B Virus (HBV), influenza, or Ebola virus.
203. The system according to embodiment 201, wherein the virus is a coronavirus.
204. The system according to embodiment 203, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).
205. The system according to embodiment 204, wherein the coronavirus is SARS-CoV-2.
206. The system according to any one of embodiments 194 to 200, wherein the pathogen is a bacterium.
207. The system according to embodiment 206, wherein the bacterium is a Staphylococcus species or a Pseudomonas species.
208. A therapeutic agent for use in identifying one or more putative escape mutations after administration of the therapeutic agent to one or more subjects for treatment of a pathogen infection, the use comprising:

obtaining a plurality of complete or partial pathogen genomic sequences isolated from one or more subjects after administration of the therapeutic agent to each subject;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

identifying, in the aligned amino acid sequences, one or more amino acid variants more frequent in the aligned amino acid sequences than in a reference, said one or more amino acid variants being one or more putative escape mutations.

209. A therapeutic agent for use in treatment of a pathogen infection, the use comprising:

selecting a conserved portion of an amino acid sequence by:
obtaining a plurality of complete or partial genomic sequences of different strains of the pathogen from a data structure;
extracting, by a processor of a computing device, coding sequences from the genomic sequences;
categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
selecting coding sequences from among the categorized coding sequences according to the measure of identity and the measure of coverage;
converting, by the processor, the selected coding sequences into corresponding amino acid sequences;
aligning, by the processor, the amino acid sequences;
classifying each of a plurality of portions of the aligned amino acid sequences according to a level of conservation of said portion among the different strains of the pathogen; and
selecting a conserved portion of the aligned amino acid sequences; and

administering the therapeutic agent to a subject if a complete or partial pathogen genomic sequence isolated from the subject encodes the conserved portion of an amino acid sequence, wherein the therapeutic agent selectively binds the conserved portion of the amino acid sequence.

210. A method of determining whether a pathogen epitope bound by an antibody is conserved, comprising:

obtaining a plurality of complete or partial genomic sequences of different strains of the pathogen from a data structure;

extracting, by a processor of a computing device, coding sequences from the genomic sequences;

comparing the coding sequences to a reference sequence encoding the pathogen epitope;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting the selected coding sequences into corresponding amino acid sequences; and

determining the level of conservation of the pathogen epitope among the different strains of the pathogen.

210. Use of a therapeutic agent for the manufacture of a medicament for identifying one or more putative escape mutations after administration of the medicament to one or more subjects for treatment of a pathogen infection, the use comprising:

obtaining a plurality of complete or partial pathogen genomic sequences isolated from one or more subjects after administration of the medicament to each subject;

extracting, by a processor of a computing device, coding sequences from the genomic sequences;

categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;

selecting coding sequences from among the categorized coding sequences according to the measure of identity and the measure of coverage;

converting, by the processor, the selected coding sequences into corresponding amino acid sequences;

aligning, by the processor, the amino acid sequences;

identifying, in the aligned amino acid sequences, one or more amino acid variants more frequent in the aligned amino acid sequences than in a reference, said one or more amino acid variants being one or more putative escape mutations.

211. Use of a therapeutic agent for the manufacture of a medicament for treatment of a pathogen infection, the use comprising:

selecting a conserved portion of an amino acid sequence by:
obtaining a plurality of complete or partial genomic sequences of different strains of the pathogen from a data structure;
extracting, by a processor of a computing device, coding sequences from the genomic sequences;
categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
selecting coding sequences from among the categorized coding sequences according to the measure of identity and the measure of coverage;
converting, by the processor, the selected coding sequences into corresponding amino acid sequences;
aligning, by the processor, the amino acid sequences;
classifying each of a plurality of portions of the aligned amino acid sequences according to a level of conservation of said portion among the different strains of the pathogen; and
selecting a conserved portion of the aligned amino acid sequences; and

administering the medicament to a subject if a complete or partial pathogen genomic sequence isolated from the subject encodes the conserved portion of an amino acid sequence, wherein the therapeutic agent selectively binds the conserved portion of the amino acid sequence.

### EXAMPLES

The present Examples provide exemplary methods and systems of the present disclosure and exemplary uses thereof. The past decade has witnessed a deluge of sequenced genomes, with viruses and bacteria, many pathogenic, among the most frequently sequenced species. For instance, according to one review of the over about 1.5 million genomic sequences present in the NCBI database, the NCBI database includes about 642,604 eukaryotic genomic sequences, about 757,524 bacterial genomic sequences, and about 176,471 viral genomic sequences.

Researchers have found, in some instances, that analysis of large-scale genomic datasets can reveal changes in pathogen genomes that correlate epidemiologically with clinical consequences. In certain examples such correlated changes may contribute significantly to pathogen phenotypes. However, as the number of publicly accessible genomic sequences rises by thousands of genomes every week, it has become increasingly difficult to manage the expanding volume of sequencing information. Moreover, accessing sequence data is not user-friendly; computational skills are required to translate the data into a workable form. The present Example provides methods and systems that extract and process publicly accessible genomic sequences. The methods and systems provided herein are particularly amenable to use in user-friendly computational programs that perform analysis of publicly accessible genomic sequences, e.g., with low or minimal user inputs.

The present Examples demonstrate the ability of analysis of publicly available genomic sequences to uncover particular characteristics of genomes that influence or are likely to influence pathogen phenotypes, e.g., host-pathogen interactions, impact therapeutic development, or provide targets for therapeutic development (e.g., development of therapeutic antibodies). The present Examples particularly demonstrate utility of the presently disclosed methods and systems in identifying, among other things, conserved sequences of use in the development of therapeutics, e.g., as antigens for therapeutic antibody development. While conventional vaccinology can require from about 5 to about 15 years for selection and validation of vaccine antigens, and reverse vaccinology using genome base approaches can require about 1 to about 2 years for selection and validation of vaccine antigens, methods and systems disclosed herein can rapidly identify antigens for vaccine development, facilitating selection and validation of vaccine antigens in about 1 to about 2 weeks, for example.

### Example 1: Exemplary Methods and Systems for Identification of Conserved Sequences of Therapeutic Interest

The present Example provides exemplary methods and systems for identification of conserved sequences of therapeutic interest. The present example utilized a computer program (“Got_Gene”) written in R, which program used BLAST algorithms known in the art and proprietary R packages to identify, compare, and characterize thousands of input genomic sequences. The Got_Gene program disclosed herein is user-friendly and does not require computational skills. It automatically interrogates public data-bases to provide a comprehensive set of information in the form of tables, graphics and visuals.

The program of the present Example included about 2,500 lines of code and 10 R packages. The program of the present Example utilized 2 to 4 external programs: BLASTn, one or both of PhyML and QuickTree, and, optionally, MegaHit. BLAST algorithms are used for alignment and are available for use, e.g., on the World Wide Web at ncbi.nlm.nih.gov; QuickTree is used for phylogeny analysis and is available for use, e.g., at HyperText Transfer Protocol github.com/tseemann/quicktree; MegaHit is used for sequence assembly and is available for use, e.g., on the World Wide Web at metagenomics.wiki/tools/assembly/megahit. R packages utilized include: data.table; IRanges; reutils; biofiles; ggplot2; cowplot; RColorBrewer; reshape2; gridExtra; DECIPHER; shiny; colourpicker; and plotly.

Without wishing to be bound by any particular exemplification or explication, the Got_Gene program used in the present Example can be viewed as having included five steps (see, e.g., FIG. 18):

(1) First, the user indicates information about the genome from which to extract the set of genes of interest. This includes selection of an organism of interest, based upon which selection genomic sequences can be identified for use as inputs (e.g., as subject inputs) in the Got_Gene program. A user can also select a list of query sequences to be used for comparative analysis;

(2) Feature and sequence files are automatically downloaded from NCBI. This includes collection of inputs (e.g., subject inputs), e.g., by download of relevant sequences from a publicly accessible database such as NCBI, including sequences optionally together with sequence annotation information;

(3) A pairwise BLAST comparison of sequences (e.g., of each query sequences with each subject sequence) provides data establishing the level of sequence diversity of each gene of interest across all genomic sequences;

(4) Data representing sequence diversity information (e.g., sequence conservation) are compiled, e.g., in a generated Got Table. A Got Table includes information about the presence or absence, level of diversity, nature of variation and genomic coordinates of each gene in each genome; and

(5) The Got Table is used to generate displays (e.g., tables, heatmaps, and/or graphs) representing compiled sequence diversity information. Generated displays can be or include a graph of sequence diversity, a maximum likelihood phylogeny, and/or alignment files. Gene sequences are then extracted from all genomes and translated to create nucleotide and amino-acid alignments. Each step is saved into fasta files. Finally, genome- and gene-based phylogenies are created using PhyML program and saved into separated files.

These steps are not intended to, and do not, limit, obviate, or require inclusion in a method or system of the present disclosure any step or series of steps provided herein.

As provided in FIG. 1, methods and systems of the present invention can include subject sequence inputs that are manually provided by a user or that are acquired from sequence databases (together with feature information such as Gff, Gbk, Gtf), and can include query sequence inputs that are manually provided by a user or that are, e.g., assembled from de novo sequencing data (e.g., Illumina or other high-throughput sequencing reads). Query and subject sequences are aligned, each query against each subject. Resulting data is used to generate GoT Tables. GoT tables can be used to generate information displays including graphics (graphs, heatmaps), sequence alignments, translated sequence alignments, and phylogeny displays (including genome-based and/or gene-based phylogeny). Genes or amino acid sequences can be selected for user-specified purposes, e.g., by identifying any of one or more, or all, of (i) most conserved genes; (ii) least conserved genes (i.e., most diverse or most variable); (iii) virulence factors; (iv) antibiotic resistance; (v) human sequence homology; (vi) secreted proteins and/or proteins including secretion domains; and (vii) transmembrane or surface proteins, and/or proteins including transmembrane or surface domains.

A first step of a method or system can be to determine characteristics of subject sequences that are to be acquired (e.g., download) (together with annotation information, if available) from one or more publicly accessible databases (e.g., NCBI) and to determine whether one or more query sequences will be manually provided for comparison to subject sequences (FIG. 2). The Got_Gene program can automatically generate certain folders for organizing and/or storing data, which folders are shown in FIG. 3.

A second step of a method or system can be to acquire subject sequences and annotation information from one or more publicly accessible databases, which can be copied to and stored in several Got_Gene folders (Reference Sequences, Aligner Databases, and Annotation Folder) (FIG. 4). Steps for acquisition of sequences and annotation information from one or more publicly accessible databases are provided in FIG. 5. The R package reutils is used to open a channel with the server of the NCBI database. Reutils is an interface to NCBI Entrez programming utilities, and provides support for a system interacting with NCBI databases such as PubMed, Gen bank, or GEO, each function of which programming interface is referred to as an R function.

A third step of a method or system can be to manually provide query sequences or download query sequences from a publicly accessible database (FIG. 6).

A fourth step of a method or system can be to align query sequences with sequences in the Aligner Databases folder (i.e., subject sequences) (FIG. 7). Steps for alignment using BLAST are provided in FIG. 8. For example, BLAST parameters for sequence comparisons can include outfmt ‘7 std sgi stitle’; minimum E-value=about 0.001; cost to open a gap=about 5; cost to extend a gap=about 2; length of best perfect match=about 11; reward for a nucleotide match=about 2; reward for a nucleotide mis-match=−about 3 (FIG. 8).

A fifth step of a method or system can include creation of a Got Table. A Got Table can include BLAST results of pairwise sequence comparisons, sequences of analyzed sequences, and available annotations (FIG. 9). BLAST outputs with no results, in that no match was identified between a particular compared pair, are discarded, including contigs without matches. BLAST results with E-values greater than about 0.001, percent identity below about 79%, or coverage length of less than about 50 nucleotides are also discarded (FIG. 10). Pairwise sequence comparisons not discarded are said to match. Where a query includes contigs and a plurality of query contigs match a particular reference sequence in an overlapping manner, it may be necessary to curate which contig is included for analysis (FIG. 11). Criteria for selecting which query contig to retain as a pairwise match of the reference sequence can include those provided in FIG. 11 (18). In generation of the Got Table, a query can be deemed present in a reference sequence if the percent of gene covered by overlapping contigs is greater than about 95%, partially present in the reference if the percent of gene covered by overlapping contigs is greater than about 80%, or absent from the reference if the percent of gene covered by overlapping contigs is less than about 79% or less than about 80% (FIG. 12). Other thresholds could also be used. For each remaining match, the SNP/size ratio can be calculated (the ratio between the number of mutations in a match and the length of that match) (FIG. 12). Single contigs that cover the entire length of a reference sequence are selected, and if multiple such contigs of a query sequence are present with respect to a reference sequence, the contig with the fewest mutations relative to the reference is retained (FIG. 12). Where no matched contig covers the entire length of a reference sequence, all contigs with a SNP/size ratio of less than about 0.5 are retained (FIG. 12). The Got Table can also incorporate annotation information (FIG. 12). A Got Table can include information relating to parameters include those shown in FIG. 13. One Got Table is generated for each query sequence (FIG. 13).

The Got Table can be used to generate a variety of information analyses and displays as outputs. One such output is a Comparative Table. To generate a Comparative Table, information on sequence similarity found in the Got Table for each query sequence as compared to all reference sequences is converted into a similarity score (FIG. 15). Similarity scores are assigned based on percent coverage of the alignment between the query and the subject, and on the number of mutations between the query and the subject. Similarity scores can be assigned, e.g., according to Table 2 (see also FIG. 14). Similarity scores can be compiled in a matrix, which matrix is the Comparative Table (FIG. 14). Similarity numbers found in the comparative table can also be presented as a heatmap, showing conservation between the relevant query and each subject sequence (FIG. 15).

Coding sequences can be identified in query nucleotide sequences based on coordinates of matches in Got Tables and associated annotations. Identified coding sequences can be extracted and translated (FIG. 16). The translated sequences can be aligned and saved in a Got_Gene folder for Extracted Sequences (FIG. 16). Where a plurality of query contigs match the reference coding sequence, overlapping contigs are merged into a single matching sequence. Query contigs that extend beyond the boundaries of the reference coding sequence may require curation (FIG. 16). The number and frequency of each variant subject coding sequence translations can be tabulated (FIG. 16). Extracted sequences can also be analyzed phylogenetically, e.g., using QuickTree (FIG. 17). Reference-based phylogenies for individual genes can be generated using reference nucleotide sequences (FIG. 17). Genome-based phylogenies for individual genomes can be generated based on the most conserved subject sequences across all query sequences, e.g., with subject sequences together including no more than about 40,000 nucleotides (FIG. 17).

The present Example demonstrate that methods and systems of the present example can be used for a variety of therapeutically relevant applications. These can include, among other things, to: (1) Determine the genetic conservation of antigens/epitopes to predict clinical potential of targeting antibodies; (2) Identify amino acid sequence variants for peptide discovery by mass-spectrometry; (3) Extract sequences and create alignments to highlight region of diversity within genes/antigens; (4) Identify regions of diversity/conservation within genomes; (5) Identify uncharacterized sequences of interest within genomes as potential therapeutic or vaccine target; (6) Build phylogenies to identify genotypes of epidemy-causing pathogens; (7) Retrieve set of orthologous genes from mis-annotated genomes; and/or (8) Differentiate relatedness in strain for epidemiological purposes.

### Example 2: Use of Methods and Systems to Identify New Therapeutic Antigens of Hepatitis B Virus

In the present Example, the Got_Gene program was used to identify new Hepatitis B virus peptides present on MHC-1 on HCC tumors, according to the methods and systems described herein. Hepatitis B virus (HBV) is a global health problem and the leading cause of hepatocellular carcinoma (HCC) (FIG. 21). People who develop a chronic infection are often treated with nucleoside analogs to suppress viral replication but are still at heightened risk of HCC. A major contributing factor to the immune system's inability to clear infection is that patients with chronic HBV have reduced numbers of HBV-specific T cells, and many of those that remain display an exhausted phenotype.

In the oncology field, T cell-redirecting antibodies have been a common approach to targeting and killing tumor cells by taking advantage of tumor-specific antigens on the surface of those cells. Unfortunately, there are no HBV proteins expressed on the surface of infected/tumor cells. However, HBV peptides complexed with MHC-I are presented on the surface of cells. Certain prior efforts had failed to identify clinically useful HBV peptides complexed with MHC-I are presented on the surface of cells. For instance, analyzing HCC tumor samples from HBV+patients, only few HBV peptides presented on the surface of cells were initially identified by mass-spectrometry. This failure was due at least in part to limiting assumptions regarding the expected sequences of such peptides. Mass spectrometry protocols uses a pre-established set of amino-acid sequences derived from a reference genome to capture the presence of peptides in an experimental set-up. Mass spectrometry is highly sensitive to peptide sequence variation and single amino acid changes between the presented-peptide and the reference sequence used to identify that peptide can have dramatic impact on signal detection. It is therefore crucial to establish the right set of reference sequences to be used for mass-spectrometry analysis.

The work described in the present Example was undertaken to identify HBV peptides complexed with MHC-I are presented on the surface of cells as new candidate HBV antigens for therapeutic antibody development, e.g., for use in development of an anti-HBV PiG/CD3 bispecific antibody to drive a T cell response against tumor/infected cells.

HBV has a circular genome of about 3.1 kb that includes about 7 overlapping coding sequences that encode about 4 polypeptides (FIG. 22). The major hepatitis B surface antigen (HBsAg) protein is encoded by gene S (FIG. 23). HBsAg is the surface antigen of HBV and is known to indicate current hepatitis B infection. Various HBV genomes are found throughout the world, and at least about 7,108 HBV genomic sequences have been published (FIG. 24). Analysis of HBV genomes by Got_Gene is demonstrative of the program's ability to analyze sequences with diverse characteristics, including circular sequences, linear sequences, fragmented sequences, DNA sequences, RNA sequences, database sequences, and manually provided sequences (FIG. 25).

In the present Example, RNAseq was performed on several HBV samples. Sequence reads were used to build a de novo genomic viral sequence for each sample. Additional HBV genomes were downloaded from NCBI (see, e.g., FIG. 18). Got_Gene was used to extract coding sequences from all HBV genomes (FIG. 26). Coding sequences of all query HBV genomes and reference HBV genomes were compared pairwise by BLAST (FIG. 27). Summary tables including resulting sequence comparison data were prepared (FIG. 28). Sequence conservation was displayed in graphs (FIG. 29), a heatmap (FIG. 30), and in phylogenies (see exemplary phylogeny displays in FIGS. 31 and 32). Extracted coding sequences (see, e.g., FIG. 34) were translated to amino acid sequences (see, e.g., FIG. 35) and amino acid sequences were aligned (see, e.g., FIG. 36). Aligned amino acid sequences were analyzed for conservation (FIG. 36).

Amino acid sequences identified in the present Example were added to the above mass spectrometry analysis protocol, enabling detection of previously unexpected HBV peptides. Mass spectrometry results were re-analyzed accordingly with updated parameters. These analyses led to the discovery of new peptides presented on the surface of infected cells. These peptides were of particular interest as they showed promiscuity to class-I human HLA binding, further supporting that they were promising targets for therapeutic development.

Got_Gene was also used to characterize the level of diversity of a potent HBV antigen across about 7,000 HBV genomes to identify highly conserved epitope regions.

### Example 3: Use of Methods and Systems to Determine Similarity Between a Sample Genome and a Collection of Reference Genomes

For historical reasons and reasons related to efficiency and conformity, a laboratory or research community will often perform experiments using one or a few particular strains of an organism of interest. These laboratory strains are often regarded as representative of non-laboratory forms (e.g., natural or wild examples of the same organism). However, there are certain drawbacks inherent in this typical approach. In particular, because the real-world diversity of a particular organism is much greater than the diversity represented by tested laboratory samples, e.g., in a given experiment, it is not necessarily the case that laboratory results are applicable across the full scope of relevant organismal diversity. To provide an example from the clinical context, a particular strain of a pathogen may be used in laboratory experiments, but clinical isolates represent a greater diversity of sequences that may or may not be adequately represented by the laboratory strain.

Methods and systems of the present disclosure can be used to determine whether a provided sequence (e.g., a genomic sequence of a laboratory strain) is characterized by sequences that are conserved (or not) among non-laboratory forms. Thus, for instance, methods and systems of the present disclosure can be applied to determine wither laboratory pathogen strains are representative of clinical isolates of the pathogen based on measured sequence conservation. Such use is particularly valuable where one or a few laboratory test strains are used in experiments intended to be representative of a broader population of strains (e.g., where one or a few strains of a pathogen may be used in the laboratory, but many different strains may be encountered in clinical application). In such scenarios, it can be important for the laboratory or test strain to be representative of a collection of reference genomes, e.g., a collection of genomes of clinical relevance.

In the present Example, Got_Gene was used to determine similarity of a sample genome and a collection of reference genomes. More specifically, Got_Gene was used to establish that a particular laboratory strain of Staphylococus aureus was representative of circulating strains causing diseases in the community. Got_Gene applied genome-based phylogeny to easily differentiate relatedness among strains for epidemiological purposes. The same approach was successfully applied to determine whether laboratory strains of Pseudomonas aeruginosa and Influenza viruses were clinically relevant.

### Example 4: Use of Methods and Systems to Evaluate Conservation of SARS-CoV-2 Receptor-Binding Domain

The coronavirus disease 2019 (COVID-19) global pandemic has motivated a widespread effort to understand adaptation mechanisms of its etiologic agent, the severe acute respiratory syndrome coronavirus 2 (SARS-CoV-2). As a result, scientists and medical professionals from around the world have sequenced the SARS-CoV-2 genome from patient isolates and disseminated their findings at unprecedented speed through curated data repositories such as the global initiative on sharing all influenza data (GISAID. Hypertext Transfer Protocol www.gisaid.org). This provided a unique dataset useful in determining transmission patterns and identifying SARS-CoV-2 variants that may be associated with virulence and disease severity.

A schematic of the structure of SARS-CoV-2 is provided in FIG. 47. It includes four structural proteins, Nucleocapsid (N) protein, Membrane (M) protein, Spike (S) protein and Envelop (E) protein and several non-structural proteins (nsp). The capsid is the protein shell of the virus. Inside the capsid, there nucleocapsid bound to the virus single positive strand RNA genome of the virus. The coronavirus genome includes about 30,000 nucleotides. Genomic sequences in RNA form can be readily converted or translated to DNA form using computational techniques and/or techniques of molecular biology.

To establish replicative niches and counter innate and adaptive immune responses, SARS-CoV-2 must adapt to host environments. A common mechanism of adaptation is antigenic variation, in which virus targets that are recognized by antibodies develop escape mutations that allow the virus to evade recognition, and elimination. The consequences of antigenic variation can include persistent viral infection, pandemics of diseases, and reinfection after recovery. In the context of COVID-19 treatment development, antigenic variation also impacts therapeutics efficacy, as emergent mutations can confound the efficacy of antibody based-treatments by modifying the protein structure of their targets.

The SARS-CoV-2 receptor-binding domain (RBD) of the viral spike protein (S) is the main target of potent neutralizing anti-S antibodies in COVID-19 patient sera or plasma samples. Therefore, S is an important target in the development of antibodies for treatment of COVID-19. Genetic conservation of the RBD is critical to ensure antibody-based treatment success, at least with respect to treatments including anti-S antibodies. In this context, Got_Gene was used to evaluate the genetic diversity of the RBD.

Since the first SARS-CoV-2 genome sequence was reported in early January 2020, there have been around 120,000 sequences deposited to GISAID as of October 2020 (Hypertext Transfer Protocol www.gisaid.org/). In the present Example, Got_Gene algorithm was used to extract, filter and compare the identity of the spike-encoding gene sequence retrieved from a total of 118,728 curated genomic sequences. In this Example, coding sequences were extracted from the reference SARS-CoV-2 genome using GenBank file annotations (illustrated in part in the schematic of FIG. 49). Pairwise comparisons were performed between each of the curated genomic sequences and the spike protein reference sequence, using BLASTn for alignment of the sequences. The cumulative number of analyzed query sequences is graphed in FIG. 50. After alignment, coding sequences aligned with the spike protein reference sequence were extracted from the curated genomic sequences. Genomic sequences that aligned with the spike protein reference sequence were then categorized based on coverage length and number of mutations as shown in Table 2. Sequences with an assigned similarity score of less than 0.8 from comparison with the spike protein reference sequence were removed from further analysis. Sequences remaining in the analysis that aligned with the spike protein reference sequences were translated into amino acid sequences and the amino acid sequences were aligned using BLASTp (illustrated in part in the schematic of FIG. 51). This analysis allowed for identification of the range of amino acids present at each aligned position of the spike protein (illustrated in part in the schematic of FIG. 52).

Results identified 965 variable amino acid positions in the SARS-CoV-2 spike protein and a total number of 1782 of unique amino-acid changes. As expected, out of the 118,728 genomes, the majority of variants were identified in only one given genome (singleton). However, 47 amino acid changes shared across more than 100 strains (high frequency variants or HFV) were identified. HFV identified within the Spike protein were found accumulating within the N-terminal and S2 domains. The RBD was spared of HFV with the exception of two HFV (N439K and S477N) identified within the receptor-binding motif which directly interacts with the human ACE2 receptor. Overall, the S protein showed relatively little sequence diversity. Among the 118,728 strains used in this study, only seven variants (LSF, L18F, R21I, A222V, S477N, D614G, and D936Y) were observed at a frequency greater than 0.6%.

One significant finding of the present Example is the strong evidence that SARS-CoV-2 epitope conservation is the rule, not the exception, in this highly successful human pathogen. The SARS-CoV-2 RBD is the main target of potent neutralizing anti-S antibodies in COVID-19 patient sera or plasma samples. Therefore, most of the selective pressure imposed by therapeutic antibodies should target this domain. Close examination of RBD conservation indicated little evidence of accumulation of mutations propagating in >0.15% of all SARS-CoV-2 strains. While several RBD variants have been identified among circulating SARS-CoV-2 isolates, none of them has reached notable frequency in the virus population as measured in this study. Altogether, these data suggest conservation of RBD-targeting antibody epitopes in circulating SARS-CoV-2; it therefore stands to reason that S-based treatment should be efficacious against all circulating SARS-CoV-2 viruses.

### Example 5: Use of Methods and Systems to Evaluate Epitope Variation

The emergence of SARS-CoV-2 in the late 2019 and its subsequent detrimental impact on human health as led to millions of infections and substantial morbidity and mortality. In an effort to stop COVID-19 pandemic, Regeneron Pharmaceuticals has applied its state of the art technologies to develop a cocktail of monoclonal antibodies dedicated to combat SARS-CoV-2 virus (see, e.g., U.S. Pat. No. 10,787,501, which is incorporated herein by reference in its entirety and particularly with respect to COVID-19 therapeutic antibodies as well as their epitopes and other properties. Table 1 of U.S. Pat. No. 10,787,501, which provides exemplary anti-SARS-CoV-2-Spike protein (SARS-CoV-2-S) antibody sequences, is specifically incorporated by reference in its entirety). Regeneron began producing hundreds of virus-neutralizing antibodies and identifying similarly-performing antibodies from human COVID-19 survivors. These antibodies specifically recognized epitopes from the receptor binding domain (RBD) of the spike protein.

Individual antibodies targeting the same antigen (e.g., SARS-CoV-2 spike protein) can have different structural targets (epitopes) within the antigen and for at least that reason can have distinct characteristics, e.g., distinct clinical performance in individual subjects and/or across a population of subjects. According to at least one approach, antibodies that bind more conserved epitopes of an antigen are preferable to antibodies that bind less conserved epitopes of an antigen, so that in any given strain or patient, or across a population of patients, the antibody is more likely to effectively bind the target antigen and/or have therapeutic effect. When a number of different antibodies are available and information is available with respect to their distinct epitopes, sequence analysis can be used to determine which antibodies advantageously bind more conserved epitopes. The present Example applies this reasoning to the development of antibodies for treatment of COVID-19. Methods and systems of the present disclosure were used to evaluate conservation of the SARS-CoV-2 epitopes of a plurality of antibodies across thousands of circulating SARS-CoV-2 strains, where antibodies targeting more conserved epitopes were selected or preferred for further therapeutic evaluation.

Comparative analysis of epitope genetic sequence across thousands of genomes was performed using the Got_Gene algorithm which allowed a quick pair-wise comparison of each genome sequence against a unique reference genome. Over 120,000 SARS-CoV-2 curated genomic sequences were extracted from the global initiative on sharing all influenza data (GISAID) database.

The SARS-CoV-2 nucleotide sequences from GISAID were aligned with the SARS-CoV-2 reference genome nucleotide sequence (GenBank accession: MN908947) using BLASTn within the Got_Gene program. Pairwise comparisons were performed between each of the curated genomic sequences and the SARS-CoV-2 reference genome sequence. After alignment, genomic sequences that aligned with the spike nucleic acid sequence of the reference SARS-CoV-2 genome were evaluated to validate presence of a spike nucleic acid sequence. Got_Gene created group categories of genomes based on determinations regarding the presence, lack of integrity, or absence of the spike protein according to certain thresholds. For each sequence, spike protein was were identified as present if comparison to the reference produced a percent coverage greater than 95%, partially present or lack of integrity if comparison to the reference produced a percent coverage greater than 70% but less than 95%, or absent if comparison to the reference produced a percent coverage of below 70%. Presence of the spike sequence was validated if comparison with the spike protein reference sequence produced a coverage length >95% and a percent identity >70%. Sequences validated according to this threshold were retained for further analysis, and all others were removed. Got_Gene extracted spike protein coding sequence from each curated genome sequence and translated validated orthologous spike sequences from each curated genome sequence into amino acid sequences. Amino acid sequences were then aligned using BLASTp and amino acid variants were identified. Epitope positions were implemented and the frequency of variants for each epitope was calculated.

### Example 6: Use of Methods and Systems to Evaluate Selection of Putative Escape Variants in Treated Subjects

The present Example demonstrates the use of methods and systems of the present disclosure to assess impact of a stimulus on sequence diversity, in particular the impact of a viral therapy on virus sequence diversity. The present Example specifically demonstrates the use of methods and systems of the present disclosure to assess impact of antibody-based COVID-19 therapy on SARS-CoV-2 sequence diversity in treatment recipients.

Two potent Regeneron antibodies (REGN10933 and REGN10987) form Regeneron's REGN-COV2 antibody therapy (see also U.S. Pat. No. 10,787,501, which is incorporated herein by reference in its entirety and particularly with respect to COVID-19 therapeutic antibodies as well as their epitopes and other properties. Table 1 of U.S. Pat. No. 10,787,501, which provides exemplary anti-SARS-CoV-2-Spike protein (SARS-CoV-2-S) antibody sequences, is specifically incorporated by reference in its entirety). In September, Regeneron announced early clinical data showing the effect of the REGN-COV2 antibody cocktail on virus genomic sequences in 275 non-hospitalized COVID-19 patients. One goal of this study was to assess the selection of putative escape variants (mutations beneficial to the virus in that they allow the virus to escape from antibody recognition) of SARS-CoV-2 isolates from patients following therapeutic administration of REGN-COV2 treatment.

In the present Example, virus genomes isolated from patients that had received REGN-COV2 treatment were sequenced, and the Got_Gene program was used to identify new mutations in the isolated genomes. Pairwise comparisons were performed between each of the isolated genomic sequences and a reference sequence encoding spike protein, using BLASTn for alignment of the sequences. After alignment, sequences that aligned with the reference sequence encoding the spike protein were extracted as query coding sequences from the curated genomic sequences. Genomic sequences that aligned with the spike protein reference sequence were then categorized based on coverage length and number of mutations as shown in Table 2. Sequences with an assigned similarity score of less than 0.8 from comparison with the spike protein reference sequence were removed from further analysis. Sequences remaining in the analysis that aligned with the spike protein reference sequences were translated into amino acid sequences and the amino acid sequences were aligned using BLASTp. This analysis allowed for identification of the range of amino acids present at each aligned position of the spike protein. Thus, Got_Gene was used to extract and translate the spike-encoding gene sequences from all genomes and compare them to the reference sequence to identify genomes in which new mutations led to amino-acid changes in the regions recognized by the neutralizing antibodies. Epitope sequence mutations can be putative escape variants. Ultimately, the analysis assessed if treatment can lead to the emergence of mutations in the SARS-CoV-2 S protein across all patient samples.

### Example 7: Use of Methods and Systems in Personalized Medicine

The present Example illustrates that methods and systems of the present disclosure can be used to select subjects likely to respond favorably to a therapeutic treatment of interest. In particular, the present Example discloses analysis of viral sequences from an infected patient to determine whether the patient would likely benefit from administration of an antibody therapy for treatment of the viral infection. For instance, the Got_Gene program can be used to identify putative escape variants in non-treated patients. The Got_Gene program can also be used to identify new mutations with putative escape potential. In this case, Got_Gene is used to extract and translate the spike-encoding gene sequences from genomes isolated from the non-treated patient to identify spike protein mutations as compared to a spike protein reference sequence, as set forth in Example 6. Identified spike protein mutations can be compared to a pre-established list of detrimental variants known or expected to negatively affect treatment efficacy. This analysis allows Got_Gene to classify patients into groups (treatment susceptible versus treatment resistant) based on the genetic background of the infecting virus strain.

### OTHER EMBODIMENTS

While we have described a number of embodiments, it is apparent that our basic disclosure and examples may provide other embodiments that utilize or are encompassed by the compositions and methods described herein. Therefore, it will be appreciated that the scope of is to be defined by that which may be understood from the disclosure and the appended claims rather than by the specific embodiments that have been represented by way of example.

All references cited herein are hereby incorporated by reference.

1. A method for identifying amino acid sequences as candidate antigens in the development of a therapy against a pathogen, comprising:
obtaining a plurality of complete or partial genomic sequences of different strains of the pathogen from a data structure;
extracting, by a processor of a computing device, coding sequences from the genomic sequences;
categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
selecting coding sequences from among the categorized coding sequences according to the measure of identity and the measure of coverage;
converting, by the processor, the selected coding sequences into corresponding amino acid sequences;
aligning, by the processor, the amino acid sequences;
classifying each of a plurality of portions of the aligned amino acid sequences according to a level of conservation of said portion among the different strains of the pathogen;
selecting portions of the amino acid sequences classified as conserved, comparing the selected conserved sequences to human protein sequences, and further classifying the selected conserved sequences as identical or not identical to a human protein sequence; and
categorizing a selected conserved sequence not identical to a human protein sequence as a candidate antigen in the development of a therapy against the pathogen.

2. The method according to claim 1, wherein the data structure comprises contigs, and wherein obtaining the plurality of complete or partial genomic sequences of different strains of the pathogen from the data structure comprises merging, by the processor, overlapping contigs to produce at least a portion of the complete or partial genomic sequences.

3. The method according to claim 1, wherein the categorizing step comprises quantifying the measure of identity and the measure of coverage for each of a plurality of pairs, each of said pairs comprising an extracted coding sequence and a reference sequence.

4. The method according to claim 1, wherein the categorizing step comprises computing, for each of a set of query coding sequences against a set of subject sequences, measures of similarity between the query coding sequence and each subject sequence, each of said measures of similarity a function of a measure of identity between the query sequence and the subject sequence and a measure of coverage between the query sequence and the subject sequence.

5. The method according to claim 4, wherein the computing step comprises creating a matrix of said measures of similarity and rendering a graphical representation of said matrix, thereby displaying levels of conservation between the query sequences and subject sequences.

6. The method according to claim 5, wherein the graphical representation comprises one or more of a heatmap, a graph, and a phylogeny.

7. The method according to claim 1, wherein the measure of identity comprises number of mutations.

8. The method according to claim 1, wherein the measure of coverage comprises percent coverage.

9. The method according to claim 1, wherein the measure of identity comprises calculating E-value.

10. The method according to claim 1, wherein categorizing the selected conserved sequence as a candidate antigen further comprises determining the presence or absence of one or more amino acid domains in the selected conserved sequence.

11. The method according to claim 1, wherein categorizing the selected conserved sequence as a candidate antigen further comprises determining whether the candidate antigen corresponds to a protein that is secreted or is exposed within a membrane and/or cell wall of the pathogen.

12. The method according to claim 1, wherein categorizing the selected conserved sequence as a candidate antigen further comprises determining the presence of a transmembrane domain in a selected conserved sequence.

13. The method according to claim 1, wherein the therapy comprises a vaccine and the method further comprises non-clinically evaluating the candidate antigen for immunogenicity.

14. The method according to claim 13, wherein the evaluating step comprises administering a polypeptide comprising the candidate antigen to an animal.

15. The method according to claim 1, wherein the therapy comprises an antibody therapy, and the method further comprises producing an antibody or fragment thereof that specifically binds to an epitope on the candidate antigen.

16. The method according to claim 1, wherein the pathogen is a virus.

17. The method according to claim 16, wherein the virus is methicillin-resistant Staphylococcus aureus (MRSA), Hepatitis B Virus (HBV), influenza, or Ebola virus.

18. The method according to claim 16, wherein the virus is a coronavirus.

19. The method according to claim 18, wherein the coronavirus is Severe Acute Respiratory Syndrome-associated coronavirus (SARS-CoV), Severe Acute Respiratory Syndrome coronavirus 2 (SARS-CoV-2), or Middle East Respiratory Syndrome-associated coronavirus (MERS-CoV).

20. The method according to claim 1, wherein the pathogen is a bacterium.

21. The method according to claim 20, wherein the bacterium is a Staphylococcus species or a Pseudomonas species.

22-46. (canceled)

47. A method of administering a therapeutic agent for treatment of a pathogen infection to a subject in need thereof, comprising:
selecting a conserved portion of an amino acid sequence by:
obtaining a plurality of complete or partial genomic sequences of different strains of the pathogen from a data structure;
extracting, by a processor of a computing device, coding sequences from the genomic sequences;
categorizing, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
selecting coding sequences from among the categorized coding sequences according to the measure of identity and the measure of coverage;
converting, by the processor, the selected coding sequences into corresponding amino acid sequences;
aligning, by the processor, the amino acid sequences;
classifying each of a plurality of portions of the aligned amino acid sequences according to a level of conservation of said portion among the different strains of the pathogen; and
selecting a conserved portion of the aligned amino acid sequences; and
administering the therapeutic agent to a subject if a complete or partial pathogen genomic sequence isolated from the subject encodes the conserved portion of an amino acid sequence, wherein the therapeutic agent selectively binds the conserved portion of the amino acid sequence.

48-179. (canceled)

180. A system for automatically identifying one or more conserved portions of coding sequences representative of a pathogen, the system comprising:
a processor; and
a memory having instructions thereon, the instructions, when executed by the processor, causing the processor to:
obtain a plurality of complete or partial genomic sequences of different strains of the pathogen from a data structure;
extract, by the processor, coding sequences from the genomic sequences;
categorize, by the processor, the coding sequences according to a measure of identity and a measure of coverage, wherein the measure of identity comprises one or more of percent identity, percent identity over a predetermined coverage length, number of mutations, and percent mutation, and wherein the measure of coverage comprises one or more of percent coverage and coverage length;
select coding sequences from among the categorized coding sequences according to the measure of identity and the measure of coverage;
convert, by the processor, the selected coding sequences into corresponding amino acid sequences;
align, by the processor, the amino acid sequences; and
classify each of a plurality of portions of the aligned amino acid sequences according to a level of conservation of said portion among the different strains of the pathogen, thereby identifying one or more conserved portions of coding sequences representative of the pathogen.

181-211. (canceled)
